(19) United States
(12) Patent Application Publication
(10) Pub. No.: US 2026/0041793 A1
(43) Pub. Date: Feb. 12, 2026

(54) VIRAL VECTOR

(71) Applicant: University of Sheffield, Sheffield (GB)

(72) Inventors: Guillaume Hautbergue, Sheffield (GB); Mimoun Azzouz, Sheffield (GB); Pamela Shaw, Sheffield (GB)

(73) Assignee: University of Sheffield, Sheffield (GB)

(21) Appl. No.: 19/101,244

(22) PCT Filed: Aug. 7, 2023
(86) PCT No.: PCT/EP2023/071868

§ 371 Date: Feb. 4, 2025

(30) Foreign Application Priority Data
Aug. 9, 2022 (GB) 2211638.8
Aug. 10, 2022 (GB) 2211673.5

(51) Int. Cl. A61K 48/00; A61K 31/7105; A61K 38/16; C07K 14/47; C12N 15/86

(52) U.S. Cl. CPC A61K 48/0058; A61K 31/7105; A61K 38/16; C07K 14/47; C12N 15/86; C12N 2310/14; C12N 2310/531; C12N 2750/14143

(57) ABSTRACT

The present disclosure relates to antagonists that target, directly or indirectly, Serine/Arginine Rich Splicing Factor 1 (SRSF1); viral vectors comprising a nucleic acid sequence encoding SRSF1 antagonists. The use of said vector in gene therapy for the treatment of neurodegenerative diseases such as for example Amyotrophic Lateral Sclerosis (ALS) or sporadic Amyotrophic Lateral Sclerosis which is not caused by a pathological C9ORF72 hexanucleotide repeat expansion and methods thereof are also disclosed.

### CROSS REFERENCE TO RELATED APPLICATIONS

This is the U.S. National Stage of International Application No. PCT/EP2023/071868, filed Aug. 7, 2023, which was published in English under PCT Article 21(2), which in turn claims the benefit of Great Britain Patent Application Nos. 2211638.8, filed Aug. 9, 2022, and 2211673.5, filed Aug. 10, 2022.

### SEQUENCES

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an XML file in the form of the file named “8617-113340-01_Sequence.xml” (192,384 bytes), which was created on Jan. 27, 2025, which is incorporated by reference herein.

### FIELD OF THE DISCLOSURE

The present disclosure relates to antagonists that target, directly or indirectly, Serine/Arginine Rich Splicing Factor 1 (SRSF1); viral vectors comprising a nucleic acid sequence encoding SRSF1 antagonists. The use of said vector in gene therapy for the treatment of neurodegenerative diseases such as for example Amyotrophic Lateral Sclerosis (ALS) or sporadic Amyotrophic Lateral Sclerosis which is not caused by a pathological C9ORF72 hexanucleotide repeat expansion and methods thereof are also disclosed.

### BACKGROUND THE DISCLOSURE

Gene therapy aims to treat diseases long-term by the introduction of genetic material which alters cell function. Several gene therapy approaches exist such as the delivery of a functional gene to replace a faulty one, inactivation of toxic genes through gene silencing or antisense, introduction or overexpression of genes absent in the host and gene editing approaches. The genetic material is most commonly delivered using viral based vectors such as adenoviruses (Ads), adeno-associated virus (AAVs), self-complementary AAVs and retroviruses i.e. lentiviruses.

The safety of gene therapy vectors requires particular attention as gene therapy vectors persist in the patient's body over a long time and gene therapy vectors must be designed to reduce genotoxic effects, immune reactions or prevent activation of adjacent genes close to the integration site. The backbone of viral vectors typically comprises the protein capsid for packaging the expressed nucleic acid, the genetic information describing the expressed nucleic acid placed between inverted terminal repeats and elements such as promoter elements which allow efficient expression in the host. When delivering genetic material of small size such as short hairpin RNA (shRNA) or antisense oligonucleotides, non-expressed “stuffer” nucleotide sequences are often required to increase the efficiency of shRNA or oligonucleotide nucleic acid targeting, expression and reach optimal packaging capacity.

Neurodegenerative diseases are typically caused by neuronal dysfunction or neuronal loss and affects millions of people worldwide. Neurodegenerative diseases are more prevalent in the aging populations and include but are not limited to amyotrophic lateral sclerosis (ALS), multiple sclerosis, Parkinson's disease, Alzheimer disease, motor neuron and Huntington's disease. ALS and frontotemporal dementia (FTD) are adult-onset neurodegenerative diseases with no effective treatment. ALS is the most common form motor neuron disease (MND), a collective term for a group of neurological disorders characterised by degeneration and loss of motor neurons. ALS is characterised by selective degeneration of the upper and lower motor neurons, leading to muscle wasting and premature death usually due to respiratory failure and paralysis. Around 90% of ALS cases are classified as sporadic, with approximately 10% showing a genetic component and familial inheritance. FTD is the second most-common form of early-onset dementia characterised by a progressive loss of neuronal cells in frontal and temporal lobe leading to alterations in cognitive function and personality.

The most common genetic cause of ALS and FTD is a hexanucleotide repeat expansion of GGGGCC in the first intron of the chromosome 9 open reading frame 72 (C9orf72) gene, termed C9ALS/FTD.

Antisense oligonucleotide therapies targeting C9ORF72 are in clinical trials and are aimed at reducing the expression of the repeat expansion, thus reducing RNA and DPR toxicity, without affecting the normal expression of C9orf72. Patent U.S. Pat. No. 10,801,027 demonstrates that depletion of the export adaptor serine/arginine-rich splicing factor 1 (SRSF1) inhibits the nuclear export of pathological C9ORF72 repeat transcripts retaining hexanucleotide repeat expansions and is hereby incorporated by reference.

However, although depletion of SRSF1 works in patients with ALS caused by hexanucleotide repeat expansions, the present disclosure identified that depletion of SRSF1 also confers neuroprotection in sporadic ALS cases which are not caused by a pathological C9ORF72 hexanucleotide repeat expansion.

### STATEMENT OF THE INVENTION

According to an aspect of the invention there is provided a viral vector comprising a transcription cassette for the expression of a nucleic acid molecule in a mammalian host cell wherein said nucleic acid molecule is operably linked to a promoter adapted to express said nucleic acid molecule in said mammalian host cell characterised in that said vector comprises a non-expressed nucleotide sequence and wherein said nucleic acid molecule encodes an antagonistic agent that targets Serin/Arginine Rich Splice Factor (SRSF1) or an SRSF1 peptide sequence.

The non-expressed nucleotide sequence is typically referred to a “Stuffer” sequence. Stuffer nucleotide sequences are known in the art and are non-expressed nucleotide sequences that provide optimal viral packaging of viral based vectors. Stuffer sequences are disclosed in PCT/US2013/031644 and is hereby incorporated by reference in its entirety. Stuffer nucleotide sequences can be placed between the viral inverted terminal repeat sequences, either side of the transgene of interest or two stuffer sequences could be added on each side of the transgene of interest.

In a preferred embodiment of the invention said antagonistic agent is a polypeptide or peptide.

In a preferred embodiment of the invention said antagonistic agent is a nucleic acid-based agent.

In a preferred embodiment of the invention said nucleic acid-based agent is an antisense nucleic acid, an inhibitory RNA or shRNA or miRNA molecule that is complementary to and inhibits the expression of a nucleic acid encoding a Serin/Arginine Rich Splice Factor (SRSF1).

Preferably said SRSF1 comprises or consist of a sequence set forth in SEQ ID NO 67.

Alternatively, said SRSF1 comprises or consist of a sequence set forth in SEQ ID NO 76.

The nucleic acid-based agent is designed with reference to the sequence set forth in SEQ ID NO 67, or alternatively with reference to the sequence set forth in SEQ ID NO 76.

In a preferred embodiment of the invention said nucleic acid-based agent is an inhibitory RNA.

In a preferred embodiment of the invention said nucleic acid-based agent is an antisense RNA.

In a further preferred embodiment of the invention said inhibitory RNA is a shRNA or miRNA molecule.

A technique to specifically ablate gene function is through the introduction of double stranded RNA, also referred to as small inhibitory or interfering RNA (siRNA, shRNA and miRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically derived from exons of the gene which is to be ablated. The mechanism of RNA interference is being elucidated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex.

The siRNA acts as a guide for the RNase complex to cleave mRNA complementary to the antisense strand of the siRNA thereby resulting in destruction of the mRNA.

In a preferred embodiment of the invention said inhibitory RNA molecule is between 19 nucleotides [nt] and 29 nt in length. More preferably still said inhibitory RNA molecule is between 21 nt and 27 nt in length. Preferably said inhibitory RNA molecule is about 21 nt in length.

In a preferred embodiment of the invention said inhibitory RNA comprises or consists of a nucleotide sequence as set forth in SEQ ID NO: 12, 13, 14, 15, 16, 17, 18, 19, 20, 21 or 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or 58.

In a preferred embodiment of the invention said shRNA comprises or consist of a nucleotide sequence selected from the group consisting of SEQ ID NO 2, 3, 4, 5, 6, 7, 8, 9, 10 and 11.

In a preferred embodiment of the invention said shRNA comprises or consist of a nucleotide sequence set forth in SEQ ID NO 7.

In a preferred embodiment of the invention said shRNA comprises or consist of a nucleotide sequence set forth in SEQ ID NO 10.

In a preferred embodiment of the invention said shRNA comprises or consist of a nucleotide sequence set forth in SEQ ID NO 11.

In a preferred embodiment of the invention said peptide comprises an amino acid sequence that is at least 10 amino acids in length and comprises all or part of the amino acid sequence set forth in SEQ ID NO: 59.

In a preferred embodiment of the invention said peptide comprises an amino acid sequence that is at least 32 amino acids in length and comprises the amino acid sequence set forth in SEQ ID NO: 59.

In a preferred embodiment of the invention said peptide is at least 10, 11, 12, 13, 14, 15, 16, 17, 18, 29, 20, 25, 30, 35, 40, 45, 50, 55, 60, 65, 70, 75, 80, 85, 90, 95 or at least 100 amino acids in length but less than the full-length amino acid sequence set forth in SEQ ID NO: 60 or 61.

In a preferred embodiment of the invention said peptide consists of an amino sequence as set forth in SEQ ID NO: 59.

In an alternative embodiment of the invention said peptide is a dominant negative protein comprising a modification of the amino acid sequence set forth in SEQ ID NO: 60 or 61.

In a preferred embodiment of the invention said dominant negative protein comprises or consists of an amino acid sequence as set forth in SEQ ID NO: 60 or 61 wherein said amino acid sequence is modified by addition, deletion or substitution of one or more amino acid residues.

In a preferred embodiment of the invention said modified protein comprises or consists of the amino acid sequence as set forth in SEQ ID NO: 62 or 63.

In a preferred embodiment of the invention said nucleic acid molecule comprising a nucleic acid sequence encoding a polypeptide or peptide is set forth set forth in SEQ ID NO: 89, or a sequence which is to 90% identical to the sequence set forth in SEQ ID NO 89.

In a further preferred embodiment of the invention said nucleic acid sequence is at least 36 nucleic acids in length.

In a preferred embodiment of the invention said peptide comprises an amino acid sequence that is at least 10, 11, 12, 13, 14, 15, 20, 25, 30, 35, 40 or 42 amino acids in length and set forth in SEQ ID NO: 90.

In a preferred embodiment of the invention said peptide comprises an amino acid sequence that is set forth in SEQ ID NO: 75 (GSWQDLKDHMREA).

In a preferred embodiment of the invention said viral vector comprises a RNA Pol Ill terminator.

Preferably said terminator comprises the nucleic acid sequence 5′ TTTTTT 3′.

In a preferred embodiment of the invention said vector comprises inverted terminal repeat nucleotide sequences.

Inverted terminal repeat sequences (ITR) are typically positioned upstream and downstream of a transcription cassette. Alternatively, the ITRs are upstream and downstream of the transcription cassette, the non-expressed nucleotide sequence and any optional regulatory elements.

In a preferred embodiment of the invention said ITR sequence is set forth in SEQ ID NO 64.

In a preferred embodiment of the invention said ITR sequence is set forth in SEQ ID NO 88.

In a preferred embodiment of the invention said promoter is selected from the group consisting of H1 Polymerase III promoter, U6 promoter, U7 promoter or the mammalian 7SK promoter.

In a further preferred embodiment of the invention said promoter is a H1 Polymerase III promoter.

In a preferred embodiment said H1 Polymerase III promoter is set forth in SEQ ID NO 65.

Viruses are commonly used as vectors for the delivery of exogenous genes. Commonly employed vectors include recombinantly modified enveloped or non-enveloped DNA and RNA viruses, for example baculoviridiae, parvoviridiae, picornoviridiae, herpesveridiae, poxviridae, adenoviridiae, picornnaviridiae or retroviridae e.g. lentivirus. Chimeric vectors may also be employed which exploit advantageous elements of each of the parent vector properties (See e.g., Feng, et al (1997) Nature Biotechnology 15:866-870). Such viral vectors may be wild-type or may be modified by recombinant DNA techniques to be replication deficient, conditionally replicating or replication competent. Conditionally replicating viral vectors are used to achieve selective expression in particular cell types while avoiding untoward broad-spectrum infection. Examples of conditionally replicating vectors are described in Pennisi, E. (1996) Science 274:342-343; Russell, and S. J. (1994) Eur. J. of Cancer 30A(8):1165-1171.

Preferred viral vectors are derived from the adenoviral, adeno-associated viral or retroviral genomes.

In a preferred embodiment of the invention said viral based vector is an adeno-associated virus [AAV].

In a preferred embodiment of the invention said adeno-associated virus is a self-complementary adeno-associated virus (scAAV).

In a preferred embodiment said viral based vector is selected from the group consisting of: AAV2, AAV3, AAV6, AAV13; AAV1, AAV4, AAV5, AAV6, AAV9 and AAVrh10.

In a preferred embodiment said scAAV is selected from the group consisting of: scAAV2, scAAV3, scAAV6, scAAV13; scAAV1, scAAV4, scAAV5, scAAV6, scAAV9 and scAAVrh10.

In a preferred embodiment of the invention said viral based vector is scAAV9 or scAAVrh10.

In an alternative preferred embodiment of the invention said viral based vector is a lentiviral vector.

According to a further aspect of the invention there is provided a pharmaceutical composition comprising a viral vector according to the invention and an excipient or carrier.

The viral vector compositions of the present invention are administered in pharmaceutically acceptable preparations. Such preparations may routinely contain pharmaceutically acceptable concentrations of salt, buffering agents, preservatives, compatible carriers and supplementary therapeutic agents. The expression vector compositions of the invention can be administered by any conventional route, including injection or by gradual infusion over time and in particular intrathecal (e.g., lumbar puncture) and/or intracerebral.

The viral vector compositions of the invention are administered in effective amounts. An “effective amount” is that amount of the expression vector that alone, or together with further doses, produces the desired response. In the case of treating a disease, the desired response is inhibiting the progression of the disease. This may involve only slowing the progression of the disease temporarily, although more preferably, it involves halting the progression of the disease permanently. This can be monitored by routine methods. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The viral vector compositions used in the foregoing methods preferably are sterile and contain an effective amount of expression vector according to the invention for producing the desired response in a unit of weight or volume suitable for administration to a patient. The doses of vector administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. Other protocols for the administration of vector compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the viral vector compositions of the invention are applied in pharmaceutically acceptable amounts and in pharmaceutically acceptable compositions. The term “pharmaceutically acceptable” means a non-toxic material that does not interfere with the effectiveness of the biological activity of the active agent. Such preparations may routinely contain salts, buffering agents, preservatives, compatible carriers, and optionally other therapeutic agents' (e.g. those typically used in the treatment of the specific disease indication). When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

The pharmaceutical compositions containing the viral vectors according to the invention may contain suitable buffering agents, including acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The viral vector compositions may conveniently be presented in unit dosage form and may be prepared by any of the methods well-known in the art of pharmacy. All methods include the step of bringing the active agent into association with a vector which constitutes one or more accessory ingredients. The preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1, 3-butanediol. Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, PA.

According to a further aspect of the invention there is provided a viral vector according to the invention for use as a medicament.

According to a further aspect of the invention there is provided a viral vector according to the invention for use in the treatment of a neurodegenerative disease.

In a preferred embodiment of the invention said neurodegenerative disease is selected from the group consisting of: amyotrophic lateral sclerosis (ALS) sporadic amyotrophic lateral sclerosis, familial ALS caused by a mutation other than a pathological C9ORF72-repeat expansion, frontotemporal dementia (FTD) motor neurone disease, frontotemporal lobar dementia (FTLD), Huntington's like disorder, and Fragile X-associated tremor/ataxia syndrome (FXTAS).

In a preferred embodiment of the invention said neurodegenerative disease is amyotrophic lateral sclerosis (ALS).

In a preferred embodiment of the invention said neurodegenerative disease is sporadic and/or familial amyotrophic lateral sclerosis.

In a preferred embodiment of the invention said neurodegenerative disease is ALS not caused by pathological C9ORF72-repeat expansions

In a preferred embodiment of the invention said neurodegenerative disease is sporadic frontotemporal dementia (FTD).

In a preferred embodiment of the invention said neurodegenerative disease is Fragile X-associated tremor/ataxia syndrome (FXTAS).

According to a further aspect of the invention there is provided a cell transfected with a viral vector according to the invention.

In a preferred embodiment of the invention said cell is a neurone and/or an astrocyte.

In a preferred embodiment of the invention said neurone is a motor neurone and/or an astrocyte.

According to a further aspect of the invention there is provided a method to treat or prevent a neurodegenerative disease comprising administering a therapeutically effective amount of a viral vector according to the invention to prevent and/or treat said neurodegenerative disease.

In a preferred method of the invention said neurodegenerative disease is sporadic amyotrophic lateral sclerosis and familial amyotrophic lateral sclerosis.

In a preferred method of the invention said neurodegenerative disease is amyotrophic lateral sclerosis.

In a preferred embodiment of the invention said neurodegenerative disease is ALS not caused by pathological C9ORF72-repeat expansions.

In a preferred method of the invention said neurodegenerative disease is sporadic frontotemporal dementia (FTD).

In a preferred method of the invention said neurodegenerative disease is Fragile X-associated tremor/ataxia syndrome (FXTAS).

According to a further aspect of the invention there is provided an isolated nucleic acid molecule encoding an shRNA molecule comprising or consisting of a nucleotide sequence selected from the group consisting of SEQ ID NO 2, 3, 4, 5, 6, 7, 8, 9, 10 and 11.

The invention includes sequence variants corresponding to the recited SEQ ID. A sequence variant is one that varies from a reference sequence by 1, 2, 3, 4 or 5 nucleotide base changes.

In a preferred embodiment of the invention said nucleic acid molecule comprises or consist of a nucleotide sequence set forth in SEQ ID NO 7.

In a preferred embodiment of the invention said nucleic acid molecule comprises or consist of a nucleotide sequence set forth in SEQ ID NO 10.

In a preferred embodiment of the invention said nucleic acid molecule comprises or consist of a nucleotide sequence set forth in SEQ ID NO 11.

According to a further aspect of the invention there is provided shRNA molecules comprising a nucleotide sequence, or variant thereof, selected from the group consisting of:

SRSF1-shRNA1 (SEQ ID NO 91):
GCUGAUGUUUACCGAGAUGGC UUCAAGAGA
GCCAUCUCGGUAAACAUCAGC;
SRSF1-shRNA2 (SEQ ID NO 92):
GGAGUUUGUACGGAAAGAAGA UUCAAGAGA
UCUUCUUUCCGUACAAACUCC;
SRSF1-shRNA3 (SEQ ID NO 93):
GGAAAGAAGAUAUGACCUAUG UUCAAGAGA
CAUAGGUCAUAUCUUCUUUCC;
SRSF1-shRNA4 (SEQ ID NO 94):
GAAAGAAGAUAUGACCUAUGC UUCAAGAGA
GCAUAGGUCAUAUCUUCUUUC;
SRSF1-shRNA5 (SEQ ID NO 95):
GCCUACAUCCGGGUUAAAGUU UUCAAGAGA
AACUUUAACCCGGAUGUAGGC;
SRSF1-shRNA6 (SEQ ID NO 96):
GGGCCCAGAAGUCCAAGUUAU UUCAAGAGA
AUAACUUGGACUUCUGGGCCC;
SRSF1-shRNA7 (SEQ ID NO 97):
GGCCCAGAAGUCCAAGUUAUG UUCAAGAGA
CAUAACUUGGACUUCUGGGCC;
SRSF1-shRNA8 (SEQ ID NO 98):
GCCCAGAAGUCCAAGUUAUGG UUCAAGAGA
CCAUAACUUGGACUUCUGGGC;
SRSF1-shRNA9 (SEQ ID NO 99):
GGAAGAUCUCGAUCUCGAAGC UUCAAGAGA
GCUUCGAGAUCGAGAUCUUCC;
and
SRSF1-shRNA10 (SEQ ID NO 100):
GCAGAGGAUCACCACGCUAUU UUCAAGAGA
AAUAGCGUGGUGAUCCUCUGC.

In a preferred embodiment of the invention said shRNA molecule comprises or consists of a nucleotide sequence, or variant thereof, set forth in SEQ ID NO 96.

In a preferred embodiment of the invention said shRNA molecule comprises or consists of a nucleotide sequence, or variant thereof, set forth in SEQ ID NO 99.

In a preferred embodiment of the invention said shRNA molecule comprises or consists of a nucleotide sequence, or variant thereof, set forth in SEQ ID NO 100.

According to an aspect of the invention there is provided an isolated nucleic acid molecule or shRNA according to the invention for use as a medicament.

According to a further aspect of the invention there is provided an isolated nucleic acid molecule or shRNA according to the invention for use in the treatment of a neurodegenerative disease.

In a preferred embodiment of the invention said neurodegenerative disease is selected from the group consisting of: amyotrophic lateral sclerosis (ALS) sporadic amyotrophic lateral sclerosis, familial ALS caused by a mutation other than a pathological C9ORF72-repeat expansion, frontotemporal dementia (FTD) motor neurone disease, frontotemporal lobar dementia (FTLD), Huntington's like disorder, and Fragile X-associated tremor/ataxia syndrome (FXTAS).

In a preferred embodiment of the invention said neurodegenerative disease is amyotrophic lateral sclerosis (ALS).

In a preferred embodiment of the invention said neurodegenerative disease is sporadic and/or familial amyotrophic lateral sclerosis.

In a preferred embodiment of the invention said neurodegenerative disease is ALS not caused by pathological C9ORF72-repeat expansions

In a preferred embodiment of the invention said neurodegenerative disease is sporadic frontotemporal dementia (FTD).

In a preferred embodiment of the invention said neurodegenerative disease is Fragile X-associated tremor/ataxia syndrome (FXTAS).

According to an aspect of the invention there is provided an siRNA molecule comprising or consisting of a nucleic acid sequence designed with reference to the shRNA set forth in SEQ ID NO 77-86.

According to an aspect of the invention there is provided an siRNA molecule according to the invention for use as a medicament.

According to a further aspect of the invention there is provided an siRNA molecule according to the invention for use in the treatment of a neurodegenerative disease.

In a preferred embodiment of the invention said neurodegenerative disease is selected from the group consisting of: amyotrophic lateral sclerosis (ALS) sporadic amyotrophic lateral sclerosis, familial ALS caused by a mutation other than a pathological C9ORF72-repeat expansion, frontotemporal dementia (FTD) motor neurone disease, frontotemporal lobar dementia (FTLD), Huntington's like disorder, and Fragile X-associated tremor/ataxia syndrome (FXTAS).

In a preferred embodiment of the invention said neurodegenerative disease is amyotrophic lateral sclerosis (ALS).

In a preferred embodiment of the invention said neurodegenerative disease is sporadic and/or familial amyotrophic lateral sclerosis.

In a preferred embodiment of the invention said neurodegenerative disease is ALS not caused by pathological C9ORF72-repeat expansions

In a preferred embodiment of the invention said neurodegenerative disease is sporadic frontotemporal dementia (FTD).

In a preferred embodiment of the invention said neurodegenerative disease is Fragile X-associated tremor/ataxia syndrome (FXTAS).

According to an aspect of the invention there is provided a cell penetrating polypeptide comprising or consisting of an amino acid sequence set forth in SEQ ID NO 90.

In a preferred embodiment of the invention said polypeptide is between 12-42 or preferably between 13-42 amino acids in length.

In a further preferred embodiment of the invention said polypeptide comprises or consist of an amino acid sequence set forth in SEQ ID NO 75.

According to an aspect of the invention there is provided a polypeptide according to the invention for use as a medicament.

According to a further aspect of the invention there is provided a polypeptide according to the invention for use in the treatment of a neurodegenerative disease.

In a preferred embodiment of the invention said neurodegenerative disease is selected from the group consisting of: amyotrophic lateral sclerosis (ALS) sporadic amyotrophic lateral sclerosis, familial ALS caused by a mutation other than a pathological C9ORF72-repeat expansion, frontotemporal dementia (FTD) motor neurone disease, frontotemporal lobar dementia (FTLD), Huntington's like disorder, and Fragile X-associated tremor/ataxia syndrome (FXTAS).

In a preferred embodiment of the invention said neurodegenerative disease is amyotrophic lateral sclerosis (ALS).

In a preferred embodiment of the invention said neurodegenerative disease is sporadic and/or familial amyotrophic lateral sclerosis.

In a preferred embodiment of the invention said neurodegenerative disease is ALS not caused by pathological C9ORF72-repeat expansions

In a preferred embodiment of the invention said neurodegenerative disease is sporadic frontotemporal dementia (FTD).

In a preferred embodiment of the invention said neurodegenerative disease is Fragile X-associated tremor/ataxia syndrome (FXTAS).

According to a further aspect of the invention there is provided an antagonistic agent comprising a nucleic acid molecule wherein said nucleic acid molecule comprises a nucleotide sequence designed with reference to human Serine/Arginine Rich Splice Factor (SRSF1) and wherein said nucleic acid molecule inhibits expression of SRSF1.

In a preferred embodiment of the invention said nucleic acid molecule is a double stranded nucleic acid molecule comprises a sense strand and an antisense strand comprising a nucleotide sequence wherein said antisense nucleotide strand is adapted to anneal by complementary base pairing to a nucleic acid molecule encoding human SRSF1.

In a preferred embodiment of the invention said double stranded nucleic acid molecule is RNA. Preferably, said RNA is siRNA or miRNA.

In an alternative embodiment of the invention said nucleic acid molecule is a single stranded nucleotide sequence comprising an antisense nucleotide sequence wherein said antisense nucleotide sequence is adapted to anneal by complementary base pairing to a nucleic acid molecule encoding SRSF1.

In a preferred embodiment of the invention said single stranded nucleic acid is DNA.

In a further preferred embodiment of the invention said single stranded nucleic acid is DNA and/or RNA.

Preferably, said DNA and/or RNA is a therapeutic antisense oligonucleotide such as an antisense oligonucleotide, a splice-switching oligonucleotide, a gapmer or similar.

Preferably said DNA is an antisense oligonucleotide.

In a preferred embodiment of the invention said nucleic acid molecule encoding human SRSF1 is set forth in SEQ ID NO: 67.

In a preferred embodiment of the invention said antagonistic agent comprises a nucleic acid molecule that is at least 15 nucleotides in length.

In a preferred embodiment of the invention said antagonistic agent comprises a nucleic acid molecule comprising a nucleotide sequence set forth in SEQ ID NO: 67 wherein said nucleic acid molecule is a double stranded inhibitory RNA and is 19-23 nucleotides in length.

In a preferred embodiment of the invention said antagonistic agent comprises a nucleic acid molecule comprises modified nucleotides.

In a preferred embodiment of the invention said double stranded nucleic acid molecule comprising sense and antisense nucleic acid molecules comprise modified nucleotides.

In a preferred embodiment of the invention said modified nucleotides/sugars are selected from the group: a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising phosphorodithioate (PS2), a nucleotide comprising a methylphosphonate group, a nucleotide comprising a 5′-phosphate, and a nucleotide comprising a 5′-phosphate mimic, for example a 5′-vinyl phosphate, a nucleotide comprising a 2′-deoxy-2′-fluro and a 2′ methyl sugar base.

In a preferred embodiment of the invention said double stranded nucleic acid molecule comprising sense and antisense nucleic acid molecules comprise modified sugar(s).

In a preferred embodiment of the invention said modified sugar is selected from the group: a modified version of the ribosyl moiety, such as —O-modified RNA such as 2′-O-alkyl or 2′-O-(substituted)alkyl e.g. 2′-O-methyl, T-O-(2-cyanoethyl), 2′-O-(2-methoxy)ethyl (2′-MOE), 2′-O-(2-thiomethyl)ethyl, 2′-O-butyryl, —O-propargyl, 2′-O-allyl, 2′-O-(2-amino)propyl, 2′-O-(2-(dimethylamino)propyl), 2′-O-(2-amino)ethyl, 2′-O-(2-(dimethylamino)ethyl); 2′-deoxy (DNA); 2′-O-(haloalkoxy)methyl, e.g. 2′-O-(2-chloroethoxy)methyl (MCEM), —O-(2,2-dichloroethoxy)methyl (DCEM); 2′-<3-alkoxycarbonyl e.g. T-O-[2-(methoxycarbonyl)ethyl](MOCE), 2′-O-[2-(N-methylcarbamoyl)ethyl](MCE), T-O-[2-(N,N-dimethylcarbamoyl)ethyl](DCME); 2′-halo e.g. 2′-F, FANA (2′-F arabinosyl nucleic acid); carbasugar and azasugar modifications; 3′-O-alkyl e.g. 3′-O-methyl, 3′-O-butyryl, V-O-propargyl and their derivatives.

In a preferred embodiment of the invention said antagonistic agent comprises or consists of a nucleotide sequence designed with reference to the target nucleic acid sequences selected from the group:

(SEQ ID NO 110)
TGGCACTGGTGTCGTGGAGTTTGTA;
(SEQ ID NO 111)
TGGTGTCGTGGAGTTTGTACGGAAA;
(SEQ ID NO 112)
TCGTGGAGTTTGTACGGAAAGAAGA;
(SEQ ID NO 113)
AAGATATGACCTATGCAGTTCGAAA;
(SEQ ID NO 114)
GAGAAACTGCCTACATCCGGGTTAA;
(SEQ ID NO 115)
CGGGTTAAAGTTGATGGGCCCAGAA;
(SEQ ID NO 116)
TGATGGGCCCAGAAGTCCAAGTTAT;
(SEQ ID NO 117)
CAGAAGTCCAAGTTATGGAAGATCT;
(SEQ ID NO 118)
GAGAAGCAGAGGATCACCACGCTAT;
and
(SEQ ID NO 119)
CGTCATAGCAGATCTCGCTCTCGTA.

In a preferred embodiment of the invention said antagonistic agent comprises a nucleic acid molecule comprising a nucleotide sequence wherein said nucleic acid molecule is a double stranded inhibitory RNA and is 19-23 nucleotides in length.

According to a further aspect of the invention there is provided a pharmaceutical composition comprising an antagonist agent according to the invention according and including an excipient or carrier.

According to a further aspect of the inventio there is provided an antagonistic agent according to the invention for use as a medicament.

According to a further aspect of the invention there is provided an antagonistic agent to the invention for use in the treatment of a neurodegenerative disease.

In a preferred embodiment of the invention said neurodegenerative disease is amyotrophic lateral sclerosis (ALS).

In a preferred embodiment of the invention said neurodegenerative disease is sporadic and/or familial amyotrophic lateral sclerosis.

In a preferred embodiment of the invention said neurodegenerative disease is ALS not caused by pathological C9ORF72-repeat expansion.

In an alternative preferred embodiment of the invention said neurodegenerative disease is sporadic frontotemporal dementia (FTD).

In an alternative preferred embodiment of the invention said neurodegenerative disease is Fragile X-associated tremor/ataxia syndrome (FXTAS).

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a aspect, embodiment or example of the invention are to be understood to be applicable to any other aspect, embodiment or example described herein unless incompatible therewith.

### BRIEF DESCRIPTION OF THE DRAWINGS

An embodiment of the invention will now be described by example only and with reference to the following figures:

FIG. 1. Timeline for differentiation and co-culture of motor neurons and astrocytes derived from healthy control and sporadic ALS (sALS) patients;

FIG. 2. Images show that MNs treated with lentivirus expressing SRSF1-miRNA retain processes/axons characteristic of neurons compared to MN treated with LV_Ctrl-miRNA which degenerate and die;

FIG. 3. Bar charts show MN survival expressed as a ratio of MNs quantified at counting day 3 over day 1 (%). 2-way ANOVA with Tukey's multiple comparison test; NS: non-significant; **: p<0.01; ***: p<0.001; ****: p<0.0001;

FIG. 4 Western immunoblotting shows that all 3 shRNAs lead to efficient depletion of SRSF1 and inhibition of the RAN translation of V5-tagged DPRs;

FIG. 5. Bar charts represents mean±sem (2-way ANOVA with Tukey's multiple comparison test; NS: non-significant; ****: p<0.0001; n=3 biological replicates). Quantification in 3 independent triplicate experiments;

FIG. 6. C9ORF72-ALS/FTD mice were injected via cisterna magna at post-natal day 1 (P1) with either 8×10¹⁰ scAAV9_Ctrl-shRNA_GFP vector genomes (vg) or 6×10¹⁰ scAAV9_SRSF1-shRNA10_GFP vg. Animals were sacrificed 1 month and 3 months post injections. Western blots show that the scAAV9_SRSF1-shRNA10_GFP virus leads to specific depletion of SRSF1 in C9ORF72-ALS/FTD mice as well as in wild type C57BL/6 mice (not shown) while the Ctrl-shRNA has no effect. GAPDH is used as a loading control;

FIGS. 7A-7N: map of scAAV_SRSF1 132-144 CPP_GFP (SEQ ID Nos: 1 and 101);

FIGS. 8A-8N: map of scAAV_SRSF1 89-120 CPP_GFP (SEQ ID Nos: 74 and 102);

FIGS. 9A-9D: (A) Western blots show depletion of SRSF1 and inhibition of the RAN translation of sense DPRs upon co-transfection with scAAV SRSF1-shRNA10_GFP, H1-CPP1_GFP and H1-CPP2_GFP, but not when CPPs transcription is driven the RNAPII promoter. SRSF1 and DPRs expression levels are quantified in triplicate biological experiments in panels B and C respectively. (D) MTT cell proliferation assays in biological triplicates showing that scAAV SRSF1-shRNA10_GFP, H1-CPP1_GFP and H1-CPP2_GFP alleviates the cytotoxicity mediated by the expression of DPRs, but not when CPPs transcription is driven the RNAPII promoter;

FIGS. 10A-10D: (A) Western blots show depletion of SRSF1 and inhibition of the RAN translation of antisense DPRs upon co-transfection with scAAV SRSF1-shRNA10_GFP, H1-CPP1_GFP and H1-CPP2_GFP, but not when CPPs transcription is driven the RNAPII promoter. SRSF1 and DPRs expression levels are quantified in triplicate biological experiments in panels B and C respectively. (D) MTT cell proliferation assays in biological triplicates showing that scAAV SRSF1-shRNA10_GFP, H1-CPP1_GFP and H1-CPP2_GFP alleviates the cytotoxicity mediated by the expression of DPRs, but not when CPPs transcription is driven the RNAPII promoter;

FIGS. 11A-11N: map of scAAV_SRSF1-shRNA10_GFP (SEQ ID NO 66 and 103);

FIGS. 12A and 12B DPR quantification in mouse brains. C9ORF72-ALS/FTD (C9-Tg) mice were injected intrathecally (via cisterna magna) with 6×10¹⁰ vector genome (vg) of scAVV9_Ctrl-shRNA_GFP or 2 doses of of scAAV9_SRSF1-shRNA10_GFP (4×10¹⁰ and 8×10¹⁰ vg) at post-natal day 1-2 (P1-2). Non-transgenic (NTg) mice are used as a control. Animals were sacrificed 1 month (A) or 3 months (B) post injection prior to MSD-ELISA quantification of poly-GP DPRs in the cerebellum/brainstem (mean±SEM; one-way ANOVA with Tukey's correction for multiple comparisons; NS: non-significant, **: p<0.01, ****: p<0.0001; n=4-6 mice/group). Poly-GP DPRs were quantified against a standard curve established with a GPx7 peptide and levels normalised to 100% for the untreated C9-Tg mice;

FIG. 13 Viral transduction in mouse brains. Immunohistochemical analysis of C9ORF72-ALS/FTD mice injected intrathecally (via cisterna magna) with 5×1010 vector genome (vg) of scAAV9_H1-SRSF1-CPP1_GFP or scAAV9_H1-CPP2_GFP at post-natal day 1-2 (P1-2). Animals were sacrificed one month post injection prior to anti-GFP immunofluorescence microscopy in the brain. Representative images are shown on the sections of midbrain. GFP co-expression is displayed in the green channel. DAPI (blue channel) and NeuN (red channel) stain nuclei and and mature neurons respectively. Side panels: Enlarged immunofluorescence images showing transduction and scAAV9-mediated co-expression of of GFP expression in both neuronal and microglial cells. Scale bars represent 500 μm; and

FIGS. 14A and 14B Viral biodistribution and DPR quantification in mouse brains. C9ORF72-ALS/FTD (C9-Tg) mice were injected intrathecally (via cisterna magna) with 5×1010 vector genome (vg) of scAAV9_H1-SRSF1-CPP1_GFP or scAAV9_H1-CPP2_GFP at post-natal day 1-2 (P1-2). Non-transgenic (NTg) mice are used as a control. Animals were sacrificed one month post injection. (A) qPCR quantification of viral DNA extracted from the brain (cerebellum) and spinal cords (n=3), showing efficient transduction. (B) MSD-ELISA quantification of poly-GP DPRs in the cerebellum/brainstem (mean±SEM; one-way ANOVA with Tukey's correction for multiple comparisons; **: p<0.01, ****: p<0.0001; N=3 mice/group). Poly-GP DPRs were quantified against a standard curve established with a GPx7 peptide and levels normalised to 100% for the untreated C9-Tg mice.

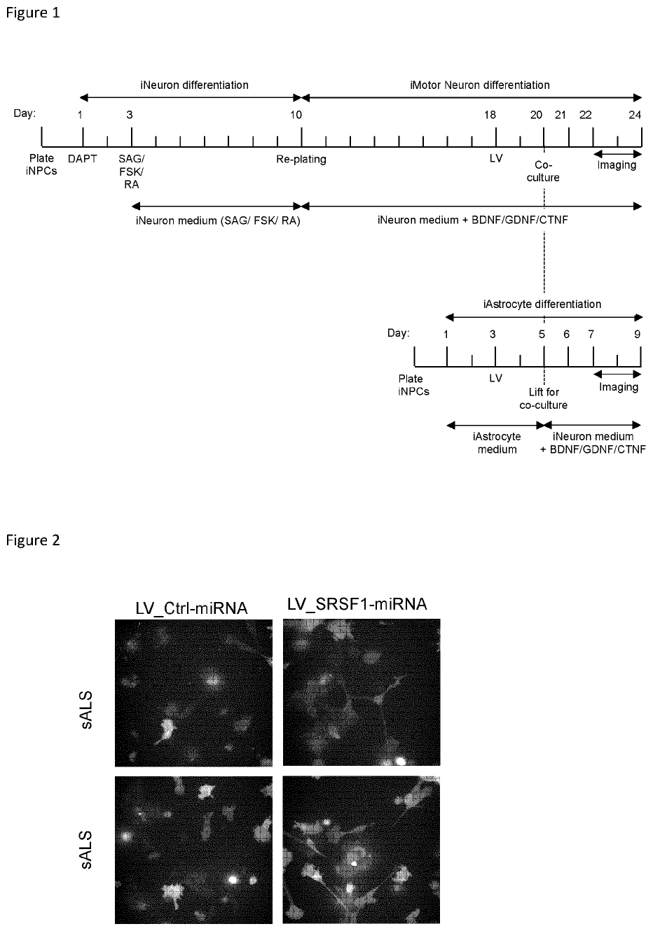

### MATERIALS AND METHODS

Part 1: SRSF1 Depletion Promotes the Survival of sALS Patient-Derived Motor Neurons Co-Cultured with Astrocytes
1/ Timeline for Differentiation and Co-Culture of Motor Neurons and Astrocytes Derived from Healthy Control and Sporadic ALS (sALS) Patients:

Summary: Both iMotor Neurons (iMNs) and iAstrocytes are treated with either 5 MOI (Multiplicity of Infection) of lentivirus (LV) expressing a Ctrl-miRNA or 2 chained miRNAs directed against SRSF1 (constructs described in Hautbergue et al, Nature Communications 2017; 8:16063 and in our patent WO2017207979A1) at day 18 and 3 of the differentiation respectively, prior to establishing co-culture from day 20 (iMN)/5 (iA). High content automated live imaging quantify iMN survival at day 22, 23, 24. scAAV9 does not efficiently transduce cells in vitro, in contrast to lentivirus which have been used here in this system.

### Detailed Protocol: Co-Cultures of Patient-Derived Astrocytes and Motor Neurons

Differentiation of iMotor Neurons (iMNs). Human patient and control-derived neurons (iNeurons) were differentiated from induced neural progenitor cells (iNPCs) using a modified version of protocol (Meyer K et al. Proc. Natl. Acad. Sci. U.S.A. 2014; 111:829-832) as previously described (Hautbergue G M et al, Nature Communications 2017; 8:16063). In brief, 100,000 iNPCs were plated in a 6-well plate coated with fibronectin (Millipore) and expanded to 70-80% confluence. Once they reached this confluence, iNPC medium was replaced with neuron differentiation medium (DMEM/F-12 with glutamax supplemented with 1% N2, 2% B27 (Gibco) containing 2.5 μM of DAPT (Tocris) to determine differentiation towards neuronal lineage on day 1. On day 3, the neuron differentiation medium was supplemented with 1 μM retinoic acid (Sigma), 0.5 μM smoothened agonist (SAG) (Millipore) and 2.5 μM forskolin (Sigma) for 7 days until Day 10. This protocol leads to typical yields of 70% β-III tubulin (Tuj1) positive cells. To obtain iMotor Neurons (iMN), ˜5,000 iNeurons per well were re-plated on 96-well plates coated with fibronectin and maintained in iNeuron differentiation medium (containing retinoic acid, SAG and forskolin) supplemented with BDNF, CNTF and GDNF (all at 20 ng/ml) for the last 14 days of differentiation.

Differentiation of iAstrocytes. Human patient-derived astrocytes (iAstrocytes) were differentiated from iNPCs as previously described (Meyer K et al. Proc. Natl. Acad. Sci. U.S.A. 2014; 111:829-832; Hautbergue G M et al, Nature Communications 2017; 8:16063) and cultured in DMEM glutamax (Gibco) with 10% FBS (Sigma) and 0.02% N2 (Invitrogen) for 5 days. Cells were maintained in a 37° C. incubator with 5% C02.

Co-cultures of patient-derived iMNs and iAstrocytes. iAstrocytes were lifted at day 5 of differentiation and ˜5,000 iAstrocytes were re-plated on iMNs at day 20 of differentiation. Co-cultured iMNs and iAstrocytes were maintained in neuron differentiation medium with BDNF, GDNF and CTNF (all at 20 ng/ml) for 4 days. 12 h after the start of co-cultures (on day 21), 1 or 10 μM CPP was added to the medium and iMNs/iAstrocytes were imaged for 72 h at days 22, 23, 24. For SRSF1 knockdown, iMNs and iAstrocytes were separately transduced 48 h prior to co-culture with lentivirus (LV) expressing control or SRSF1-RNAi co-expressing GFP (Hautbergue G M et al, Nature Communications 2017; 8:16063) at a MOI of 5 at day 18 of iMN differentiation and at day 3 of iAstrocyte differentiation.

### Part 2: ScAAV9-Driven Expression of SRSF1-shRNA

### Pre-Clinical Vector Design: ScAAV_SRSF1-shRNA_GFP

### 1/ SRSF1-shRNA Cassette Targeting Mouse, Rat, Non-Human Primate and Human SRSF1

Take region human SRSF1 448-750 (3′ end of open reading frame) which is highly conserved with mouse SRSF1.

Human SRSF1 (NM_006924.4)
SEQ ID NO 67
gctgatgtttaccgagatggcactggtgtcgtggagtttgtacggaaagaagatatgacctatgcagttcgaaaactggataacac
taagtttagatctcatgagggagaaactgcctacatccgggttaaagttgatgqgcccagaagtccaagttatggaagatctcgat
ctcgaagccgtagtcgtagcagaagccgtagcagaagcaacagcaggagtcgcagttactccccaaggagaagcagagga
tcaccacgctattctccccgtcatagcagatctcgctctcgtacataa
(SEQ ID NO 87)
ttaaagttgatgggcccagaa miRNA used to target human and mouse SRSF1 in
the lentivirus construct (Hautbergue et al. Nature Communications 2017; 8:16063
and in our patent WO2017207979A1)

Design shRNA using the following website:

Block-iT RNAi Designer tool: rnaidesigner.lifetechnologies.com/rnaiexpress/

TABLE 1
SEQ ID NO 2-11

| | | | | | Rank (predicted |
|---|---|---|---|---|---|
| No. | Start | (nt) Target sequence (DNA) | Region | GC% | efficacy 0-5) |
| 2 | 1 | GCTGATGTTTACCGAGATGGC | 52.39 | | 3.5 |
| 3 | 33 | GGAGTTTGTACGGAAAGAAGA | 42.86 | | 4.5 |
| 4 | 44 | GGAAAGAAGATATGACCTATG | 38.1 | | 3.5 |
| | | not fully conserved | | | |
| | | human/mouse | | | |
| 5 | 45 | GAAAGAAGATATGACCTATGC | 38.1 | | 3.5 |
| | | not fully conserved | | | |
| | | human/mouse | | | |
| 6 | 115 | GCCTACATCCGGGTTAAAGTT | 47.62 | | 3.5 |
| 7 | 139 | GGGCCCAGAAGTCCAAGTTAT | 52.39 | | 4.5 |
| 8 | 140 | GGCCCAGAAGTCCAAGTTATG | 52.39 | | 3.5 |
| 9 | 141 | GCCCAGAAGTCCAAGTTATGG | 52.39 | | 4.0 |
| 10 | 160 | GGAAGATCTCGATCTCGAAGC | 52.39 | | 4.5 |
| 11 | 245 | GCAGAGGATCACCACGCTATT | 52.39 | | 5.0 |

TABLE 2
Use siSPOTR (Boudreau RL et al. Nucleic Acids Res. 2013; 41(1):e9) to predict the
off target of the common human/mouse sequences targeting SRSF1 and predicted most
efficient. Table 2 depicts SEQ ID NO: 77-86.

| shRNA | antisense/ mature | | | |
|---|---|---|---|---|
| sequence | shRNA sequence | POTS | POTS | Seed |
| 77 | gccaucucgguaaacaucagc | 355.351 (mouse) | 463.716 (human) | CCATCTC |
| 78 | ucuucuuuccguacaaacucc | 501.411 (mouse) | 588.488 (human) | CTTCTTT |
| 79 | cauaggucauaucuucuuucc | 96.137 (mouse) | 149.126 (human) | ATAGGTC |
| 80 | gcauaggucauaucuucuuuc | 140.373 (mouse) | 167.649 (human) | CATAGGT |
| 81 | aacuuuaacccggauguaggc | 390.256 (mouse) | 526.984 (human) | ACTTTAA |
| 82 | auaacuuggacuucugggccc | 221.397 (mouse) | 339.052 (human) | TAACTTG |
| 83 | cauaacuuggacuucugggcc | 237.138 (mouse) | 351.458 (human) | ATAACTT |
| 84 | ccauaacuuggacuucugggc | 159.58 (mouse) | 215.339 (human) | CATAACT |
| 85 | gcuucgagaucgagaucuucc | 41.326 (mouse) | 41.3938 (human) | CTTCGAG |
| 86 | aauagcguggugauccucugc | 21.324 (mouse) | 22.5396 (human) | ATAGCGT |

SRSF1 target shRNA6 sequence:
(SEQ ID NO 7)
5′-GGGCCCAGAAGTCCAAGTTAT-3′
Antisense/mature shRNA6 sequence:
(SEQ ID NO 82)
5′-AUAACUUGGACUUCUGGGCCC-3′
SRSF1 target shRNA9 sequence:
(SEQ ID NO 10)
5′-GGAAGATCTCGATCTCGAAGC-3′
Antisense/mature shRNA9 sequence:
(SEQ ID NO 85)
5′-GCUUCGAGAUCGAGAUCUUCC-3′
SRSF1 target shRNA10 sequence:
(SEQ ID NO 11)
5′-GCAGAGGATCACCACGCTATT-3′
Antisense/mature shRNA10 sequence:
(SEQ ID NO 86)
5′-AAUAGCGUGGUGAUCCUCUGC-3′

### 2/ Alignment Human (NM_006924.4; SEQ ID NO 104) and Mouse (NM_173374.4; SEQ ID NO 105) SRSF1

Sequences corresponding to the shRNAs 7, 10 and 11 (predicted the most efficient with the less predicted off-target effects) are indicated on the aligned human and mouse SRSF1 open reading frames.

| | | |
|---|---|---|
| hSRSF1 | ATGTCGGGAGGTGGTGTGATTCGTGGCCCCGCAGGGAACAACGATTGCCGCATCTACGTG | 60 |
| | \|\|\|\|\|\|\|\|\|\|\|\|\|\|\|\|\|\|\|\| \|\|\|\|\|\|\|\| \|\| \|\|\|\|\|\|\|\|\|\|\| \|\|\|\|\|\|\|\|\|\|\|\|\|\|\| | |
| mSRSF1 | ATGTCGGGAGGTGGTGTGATCCGTGGCCCGGCGGGGAACAACGACTGCCGCATCTACGTG | 60 |
| hSRSF1 | GGTAACTTACCTCCAGACATCCGAACCAAGGACATTGAGGACGTGTTCTACAAATACGGC | 120 |
| | \|\|\|\|\|\| \|\|\|\|\|\|\| \|\| \|\|\|\|\|\|\|\|\|\|\|\|\|\|\|\|\| \|\|\|\|\|\|\|\|\|\|\| \|\|\|\|\|\|\|\|\|\|\|\| | |
| mSRSF1 | GGTAACCTACCTCCGGATATCCGAACCAAGGACATCGAGGACGTGTTTTACAAATACGGC | 120 |
| hSRSF1 | GCTATCCGCGACATCGACCTCAAGAATCGCCGCGGGGGACCGCCCTTCGCCTTCGTTGAG | 180 |
| | \|\| \|\|\|\|\|\|\|\|\|\|\|\|\|\|\|\|\| \|\|\|\|\| \|\|\|\|\|\|\|\|\|\|\|\|\|\|\|\|\|\|\|\|\|\|\|\|\|\|\|\|\|\|\|\|\| | |
| mSRSF1 | GCCATCCGCGACATCGACCTGAAGAACCGCCGCGGGGGACCGCCCTTCGCCTTCGTTGAG | 180 |
| hSRSF1 | TTCGAGGACCCGCGAGACGCGGAAGACGCGGTGTATGGTCGCGACGGCTATGATTACGAT | 240 |
| | \|\|\|\|\|\|\|\|\|\|\|\|\|\|\|\|\|\|\|\|\|\|\|\|\|\| \|\|\|\|\|\|\|\| \|\|\|\|\|\|\|\|\|\|\|\|\|\| \|\| \|\|\|\|\| | |
| mSRSF1 | TTCGAGGACCCGCGAGACGCGGAAGATGCGGTGTACGGTCGCGACGGCTACGACTACGAC | 240 |
| hSRSF1 | GGGTACCGTCTGCGGGTGGAGTTTCCTCGAAGCGGCCGTGGAACAGGCCGAGGCGGCGGC | 300 |
| | \|\| \|\|\|\|\| \|\|\|\|\|\|\|\| \|\|\|\|\|\|\|\| \|\|\|\|\|\|\|\|\|\|\| \|\| \|\| \|\|\|\|\|\|\|\|\|\|\|\|\|\|\| | |
| mSRSF1 | GGCTACCGGCTGCGGGTAGAGTTTCCCCGAAGCGGCCGCGGGACCGGCCGAGGCGGCGGC | 300 |
| hSRSF1 | GGGGGTGGAGGTGGCGGAGCTCCCCGAGGTCGCTATGGCCCCCCATCCAGGCGGTCTGAA | 360 |
| | \|\|\|\|\|\|\|\|\|\|\| \|\|\|\|\| \|\| \|\| \|\|\|\|\|\|\|\|\|\|\|\|\|\|\|\| \|\| \|\|\|\|\|\|\|\|\|\|\| \|\| | |
| mSRSF1 | GGGGGTGGAGGCGGCGGCGCCCCGAGAGGCCGCTATGGCCCGCCGTCCAGGCGGTCCGAG | 360 |
| hSRSF1 | AACAGAGTGGTTGTCTCTGGACTGCCTCCAAGTGGAAGTTGGCAGGATTTAAAGGATCAC | 420 |
| | \|\|\|\|\|\|\|\|\|\|\|\|\|\|\|\|\|\|\|\|\|\|\|\|\|\|\|\|\| \|\|\|\|\|\|\|\| \|\|\|\|\|\|\|\| \|\|\|\|\|\|\|\|\|\|\|\| | |
| mSRSF1 | AACAGAGTGGTTGTCTCTGGACTGCCTCCGAGTGGAAGCTGGCAGGACTTAAAGGATCAC | 420 |
| hSRSF1 | ATGCGTGAAGCAGGTGATGTATGTTATGCTGATGTTTACCGAGATGGCACTGGTGTCGTG | 480 |
| | \|\|\|\|\|\|\|\| \|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\| | |
| mSRSF1 | ATGCGTGAGGCAGGTGATGTATGTTACGCTGATGTTTACCGAGATGGCACTGGTGTCGTG | 480 |
| hSRSF1 | GAGTTTGTACGGAAAGAAGATATGACCTATGCAGTTCGAAAACTGGATAACACTAAGTTT | 540 |
| | \|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\| | |
| mSRSF1 | GAGTTTGTACGGAAAGAAGATATGACGTATGCAGTTCGAAAACTGGATAACACTAAGTTT | 540 |
| hSRSF1 | AGATCTCATGAGGGAGAAACTGCCTACATCCGGGTTAAAGTTGATGGGCCCAGAAGTCCA | 600 |
| | \|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| mSRSF1 | AGATCTCACGAGGGAGAAACTGCCTACATCCGGGTTAAAGTTGATGGGCCCAGAAGTCCA | 600 |
| hSRSF1 | AGTTATGGAAGATCTCGATCTCGAAGCCGTAGTCGTAGCAGAAGCCGTAGCAGAAGCAAC | 660 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| mSRSF1 | AGTTATGGAAGATCTCGATCTCGAAGCCGTAGTCGTAGCAGAAGCCGTAGCAGAAGCAAC | 660 |
| hSRSF1 | AGCAGGAGTCGCAGTTACTCCCCAAGGAGAAGCAGAGGATCACCACGCTATTCTCCCCGT | 720 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| mSRSF1 | AGCAGGAGTCGCAGTTACTCCCCAAGGAGAAGCAGAGGATCACCACGCTATTCTCCCCGT | 720 |
| hSRSF1 | CATAGCAGATCTCGCTCTCGTACATAA | 747 |
| | \|\|\|\|\|\|\|\|\|\|\|\|\|\|\|\|\|\|\|\|\|\|\|\|\|\|\| | |
| mSRSF1 | CATAGCAGATCTCGCTCTCGTACATAA | 747 |

SEQ ID NO: 82 corresponds to the aspect in bold above (residues 585-606 of SEQ ID
NO: 104 or SEQ ID NO: 105). SEQ ID NO: 85 corresponds to the aspect in italics
above (residues 607-627 of SEQ ID NO: 104 or SEQ ID NO: 105). SEQ ID NO: 86
corresponds to the aspect in bold and italics above (residues 691-711 of SEQ ID NO:
104 or SEQ ID NO: 105).

| | antisense/mature | | | |
|---|---|---|---|---|
| shRNA | shRNA sequence | POTS | POTS | Seed sequence |
| 82 | auaacuuggacuucugggccc | 221.397 (mouse) | 339.052 (human) | TAACTTG |
| 85 | gcuucgagaucgagaucuucc | 41.326 (mouse) | 41.3938 (human) | CTTCGAG |
| 86 | aauagcguggugauccucugc | 21.324 (mouse) | 22.5396 (human) | ATAGCGT |

3/ Cloning of SRSF1-Targeting shRNAs into the scAAV_GFP Vector

We then designed and custom synthesised the following oligonucleotides for cloning shRNAs 7, 10 and 11 into our scAAV_H1promoter_GFP vector (SEQ ID NO 68-73):

SRSF1 shRNA_6_fwd
(SEQ ID NO 68)
GATCC GGGCCCAGAAGTCCAAGTTAT TTCAAGAGA ATAACTTGGACTTCTGGGCCC C
TTTTTT GGA A

Residues 1-5 in SEQ ID NO: 68 correspond to cut BamHI. Residues 6-26 in SEQ ID NO: 68 correspond to SRSF1 targeted region. Residues 27-35 in SEQ ID NO: 68 correspond to hairpin loop. Residues 36-56 in SEQ ID NO: 68 correspond to antisense/mature shRNA. Residues 57-66 in in SEQ ID NO: 68 correspond to hairpin loop. Residue 67 in SEQ ID NO: 68 corresponds to cut HindIII.

SRSF1 shRNA_6_rev (SEQ ID NO 69):
AGCTT TCC AAAAAA G GGGCCCAGAAGTCCAAGTTAT TCTCTTGAA
ATAACTTGGACTTCTGGGCCC G

Residues 1-5 in SEQ ID NO: 69 correspond to cut HindIII. Residues 6-15 in SEQ ID NO: 69 correspond to hairpin loop. Residues 16-36 in SEQ ID NO: 69 correspond to SRSF1 targeted region. Residues 37-45 in SEQ ID NO: 69 correspond to hairpin loop. Residues 46-66 in SEQ ID NO: 69 correspond to antisense/mature shRNA. Residue 67 in SEQ ID NO: 69 corresponds to cut BamHI.

SRSF1 shRNA_9_fwd (SEQ ID NO 70):
GATCC GGAAGATCTCGATCTCGAAGC TTCAAGAGA GCTTCGAGATCGAGATCTTCC C
TTTTTT GGA A

Residues 1-5 in SEQ ID NO: 70 correspond to cut BamHI. Residues 6-26 in SEQ ID NO: 70 correspond to SRSF1 targeted region. Residues 27-35 in SEQ ID NO: 70 correspond to hairpin loop. Residues 36-56 in SEQ ID NO: 70 correspond to antisense/mature shRNA. Residues 57-66 in SEQ ID NO: 70 correspond to hairpin loop. Residue 67 in SEQ ID NO: 70 corresponds to cut HindIII.

SRSF1 shRNA_9_rev (SEQ ID NO 71):
AGCTT TCC AAAAAA G GGAAGATCTCGATCTCGAAGC TCTCTTGAA
GCTTCGAGATCGAGATCTTCC G

Residues 1-5 in SEQ ID NO: 71 correspond to cut HindIII. Residues 6-15 in SEQ ID NO: 71 correspond to hairpin loop. Residues 16-36 in SEQ ID NO: 71 correspond to SRSF1 targeted region. Residues 37-45 in SEQ ID NO: 71 correspond to hairpin loop. Residues 46-66 in SEQ ID NO: 71 correspond to antisense/mature shRNA. Residue 67 in SEQ ID NO 71 corresponds to cut BamHI.

SRSF1 shRNA 10_fwd (SEQ ID NO 72):
GATCC GCAGAGGATCACCACGCTATT TTCAAGAGA AATAGCGTGGTGATCCTCTGC C
TTTTTT GGA A

Residues 1-5 in SEQ ID NO: 72 correspond to cut BamHI. Residues 6-26 in SEQ ID NO: 72 correspond to SRSF1 targeted region. Residues 27-35 in SEQ ID NO: 72 correspond to hairpin loop. Residues 36-56 in SEQ ID NO 72 correspond to antisense/mature shRNA. Residues 57-66 in SEQ ID NO: 72 correspond to hairpin loop. Residue 67 in SEQ ID N: 72 corresponds to cut HindIII.

SRSF1 shRNA_10_rev (SEQ ID NO 73):
AGCTT TCC AAAAAA G GCAGAGGATCACCACGCTATTT CTCTTGAA
AATAGCGTGGTGATCCTCTGC G

Residues 1-5 in SEQ ID NO: 73 correspond to cut HindIII. Residues 6-15 in SEQ ID NO: 73 correspond to hairpin loop. Residues 16-36 in SEQ ID NO 73 correspond to SRSF1 targeted region. Residues 37-45 in SEQ ID NO: 73 correspond to hairpin loop. Residues 46-66 in SEQ ID NO: 73 corresponds to antisense/mature shRNA. Residue 67 in SEQ ID NO: 73 corresponds to cut BamHI.

5/ Full Sequence of the Pre-Clinical scAAV Vector Co-Expressing the SRSF1-shRNA Cassette (Under Constitutively-Expressed RNAPIII H1 Promoter) and eGFP (Under a Weak RNAPII eF-1Alpha Core Promoter to Avoid Potential GFP-Induced Toxicity)

SCAAV_SRSF1-shRNA10_GFP circular sequence (5,648 bp)
SEQ ID NO 66
5′...
GCTCTTCCGCTTCCTCGCTCACTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCGCCCCCCTGACGAG
CATCACAAAAATCGACGCTCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCCCAGTGCTGCAA
TGATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTGGTGTCACGCTCGTCGTT
TGGTATGGCTTCATTCAGCTCCGGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAGGATCTT
ACCGCTGTTGAGATCCAGTTCGATGTAACCCACTCGTGCACCCAACTGATCTTCAGCATCTTTTACTTTCACC
AGCGTTTCTGGGTGAGCAAAAACAGGAAGGCAAAATGCCGCAAAAAAGGGAATAAGGGCGACACGGAAATG
TTGAATACTCATACTCTTCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCCACCTGACGT
CTAAGAAACCATTATTATCATGACATTAACCTATAAAAATAGGCGTATCACGAGGCCCTTTCGTCTCGCGCGT
TTCGGTGATGACGGTGAAAACCTCTGACACATGCAGCTCCCGGAGACGGTCACAGCTTGTCTGTAAGCGGA
TGCCGGGAGCAGACAAGCCCGTCAGGGCGCGTCAGCGGGTGTTGGCGGGTGTCGGGGCTGGCTTAACTA
TGCGGCATCAGAGCAGATTGTACTGAGAGTGCACCATAGTGTTGGCGGGTGTCGGGGCTGGCTTAACTATG
CGGCATCAGAGCAGATTGTACTGAGAGTGCACCATATGCGGTGTGAAATACCGCACAGATGCGTAAGGAGA
AAATACCGCATCAGGCGATTCCAACATCCAATAAATCATACAGGCAAGGCAAAGAATTAGCAAAATTAAGCAA
TAAAGCCTCAGAGCATAAAGCTAAATCGGTTGTACCAAAAACATTATGACCCTGTAATACTTTTGCGGGAGAA
GCCTTTATTTCAACGCAAGGATAAAAATTTTTAGAACCCTCATATATTTTAAATGCAATGCCTGAGTAATGTGT
AGGTAAAGATTCAAACGGGTGAGAAAGGCCGGAGACAGTCAAATCACCATCAATATGATATTCAACCGTTCT
AGCTGATAAATTCATGCCGGAGAGGGTAGCTATTTTTGAGAGGTCTCTACAAAGGCTATCAGGTCATTGCCT
GAGAGTCTGGAGCAAACAAGAGAATCGCCGGGGGGGGGGGGGGGGGGGGCCACTCCCTCTCTGCGCGCT
CGCTCGCTCACTGAGGCCGCCCGGGCAAAGCCCGGGCGTCGGGCGACCTTTGGTCGCCCGGCCTCAGTG
AGCGAGCGAGCGCGCAGAGAGGGAGTGGCCAACTCCATCACTAGGGGTTCCTCAGATCGATCTCTCCCCA
GCATGCAGGCCTCTGCAGTCGACGGGCCCGGCATGCGTTTTACTCCCCAGCATGCCTGCTATTCTCTTCCC
AATCCTCCCCCTTGCTGTCCTGCCCCACCCCACCCCCCAGAATAGAATGACACCTACTCAGACAATGCGATG
CAATTTCCTCATTTTATTAGGAAAGGACAGTGGGAGTGGCACCTTCCAGGGTCAAGGAAGGCACGGGGGAG
GGGCAAACAACAGATGGCTGGCAACTAGAAGGCACAGTCGAGGCTGATCAGCGAGCTCTAGGAATTTTACT
TGTACAGCTCGTCCATGCCGAGAGTGATCCCGGCGGCGGTCACGAACTCCAGCAGGACCATGTGATCGCG
CTTCTCGTTGGGGTCTTTGCTCAGGGCGGACTGGGTGCTCAGGTAGTGGTTGTCGGGCAGCAGCACGGGG
CCGTCGCCGATGGGGGTGTTCTGCTGGTAGTGGTCGGCGAGCTGCACGCTGCCGTCCTCGATGTTGTGGC
GGATCTTGAAGTTCACCTTGATGCCGTTCTTCTGCTTGTCGGCCATGATATAGACGTTGTGGCTGTTGTAGTT
GTACTCCAGCTTGTGCCCCAGGATGTTGCCGTCCTCCTTGAAGTCGATGCCCTTCAGCTCGATGCGGTTCA
CCAGGGTGTCGCCCTCGAACTTCACCTCGGCGCGGGTCTTGTAGTTGCCGTCGTCCTTGAAGAAGATGGTG
CGCTCCTGGACGTAGCCTTCGGGCATGGCGGACTTGAAGAAGTCGTGCTGCTTCATGTGGTCGGGGTAGC
GGCTGAAGCACTGCACGCCGTAGGTCAGGGTGGTCACGAGGGTGGGCCAGGGCACGGGCAGCTTGCCGG
TGGTGCAGATGAACTTCAGGGTCAGCTTGCCGTAGGTGGCATCGCCCTCGCCCTCGCCGGACACGCTGAA
CTTGTGGCCGTTTACGTCGCCGTCCAGCTCGACCAGGATGGGCACCACCCCGGTGAACAGCTCCTCGCCC
TTGCTCACCATGCCTGTGTTCTGGCGGCAAACCCGTTGCGAAAAAGAACGTTCACGGCGACTACTGCACTTA
TATACGGTTCTCCCCCACCCTCGGGAAAAAGGCGGAGCCAGTACACGACATCACTTTCCCAGTTTACCCCG
CGCCACCTTCTCTAGGCACCGGATCAATTGCCGACCCCTCCCCCCAACTTCTCGGGGACTGTGGGCGATGT
GCGCTCTGCCCACTGAATCTTCTCGAGCCTCTAGATACCACAGGCTGCGCAACTGTTGGGAAGGGCGATCG
GTGCGGGCCTCTTCGCTATTACGCCAGCTGGCGAAAGGGGGATGTGCTGCAAGGCGATTAAGTTGGGTAA
CGCCAGGGTTTTCCCAGTCACGACGTTGTAAAACGACGGCCAGTGAATTCATATTTGCATGTCGCTATGTGT
TCTGGGAAATCACCATAAACGTGAAATGTCTTTGGATTTGGGAATCTTATAAGTTCTGTATGAGACCACTCGG
ATCCGCAGAGGATCACCACGCTATTTTCAAGAGAAATAGCGTGGTGATCCTCTGCCTTTTTTGGAAAGCTTG
GCGTAATCATGGTCATAGCTGTTTCCTGTGTGAAATTGTTATCCGCTCACAATTCCACACAACATACGAGCCG
GAAGCATAAAGTGTATCTAGAGCGGTACCACGCGTGAATTGAATTCAGATCCACGCGTGAATTCCACTCCCT
CTCTGCGCGCTCGCTCGCTCACTGAGGCCGGGCGACCAAAGGTCGCCCGACGCCCGGGCTTTGCCCGGG
CGGCCTCAGTGAGCGAGCGAGCGCGCAGGGCGATGAACGGTAATCGTAAAACTAGCATGTCAATCATATGT
ACCCCGGTTGATAATCAGAAAAGCCCCAAAAACAGGAAGATTGTATAAGCAAATATTTAAATTGTAAGCGTTA
ATATTTTGTTAAAATTCGCGTTAAATTTTTGTTAAATCAGCTCATTTTTTAACCAATAGGCCGAAATCGGCAAA
ATCCCTTATAAATCAAAAGAATAGACCGAGATAGGGTTGAGTGTTGTTCCAGTTTGGAACAAGAGTCCACTAT
TAAAGAACGTGGACTCCAACGTCAAAGGGCGAAAAACCGTCTATCAGGGCGATGGCCCACTACGTGAACCA
TCACCCTAATCAAGTTTTTTGGGGTCGAGGTGCCGTAAAGCACTAAATCGGAACCCTAAAGGGAGCCCCCG
ATTTAGAGCTTGACGGGGAAAGCCGGCGAACGTGGCGAGAAAGGAAGGGAAGAAAGCGAAAGGAGCGGG
CGCTAGGGCGCTGGCAAGTGTAGCGGTCACGCTGCGCGTAACCACCACACCCGCCGCGCTTAATGCGCCG
CTACAGGGCGCGTACTATGGTTGCTTTGACGAGCACGTATAACGTGCTTTCCTCGTTAGAATCAGAGCGGG
AGCTAAACAGGAGGCCGATTAAAGGGATTTTAGACAGGAACGGTACGCCAGAATCCTGAGAAGTGTTTTTAT
AATCAGTGAGGCCACCGAGTAAAAGAGTCTGTCCATCACGCAAATTAACCGTTGTCGCAATACTTCTTTGATT
AGTAATAACATCACTTGCCTGAGTAGAAGAACTCAAACTATCGGCCTTGCTGGTAATATCCAGAACAATATTA
CCGCCAGCCATTGCAACGGAATCGCCATTCGCCATTCAGGCTGCGCAACTGTTGGGAAGGGCGATCGGTG
CGGGCCTCTTCGCTATTACGCCAGCTGCATTAATGAATCGGCCAACGCGCGGGGAGAGGCGGTTTGCGTAT
TGGGC...3′

6/ Functionality of scAAV9-Driven Expression of SRSF1-shRNA10 in Mouse Brains.

C9ORF72-ALS/FTD mice were injected via cisterna magna at post-natal day 1 (P1) with either 8×10¹⁰ scAAV9_Ctrl-shRNA_GFP vector genomes (vg) or 6×10¹⁰ scAAV9_SRSF1-shRNA10_GFP vg. Animals were sacrificed 1 month and 3 months post injections. Western blots show that the scAAV9_SRSF1-shRNA10_GFP virus leads to specific depletion of SRSF1 in C9ORF72-ALS/FTD mice as well as in wild type C57B16 mice (not shown) while the Ctrl-shRNA has no effect. GAPDH is used as a loading control.

4/ITR sequences
SEQ ID NO 64:
ITR1:
5′-ccactccctctctgcgcgctcgctcgctcactgaggccgcccgggcaaagcccgggcgtcgggcgacctttggtcgc
ccggcctcagtgagcgagcgagcgcgcagagagggagtggccaactccatcactaggggttcct-3′
SEQ ID NO 88:
ITR2:
5′-ccactccctctctgcgcgctcgctcgctcactgaggccgggcgaccaaaggtcgcccgacgcccgggctttg
cccgggcggcctcagtgagcgagcgagcgcgcag-3′

### Sequence of Cell Permeable Peptide

SRSF1 132-144 CPP nucleotide sequence:
(SEQ ID NO 89)
5′-GGCAGCTGGCAGGATCTGAAAGATCATATGCGCGAAGCCGGCGGTGGGAAACCGATTCCCAACCCGCTGC
TGGGCCTCGATAGCACCGGCGGATATGGTCGCAAAAAGCGCAGACAGCGCCGGAGG-3′
SRSF1 132-144 CPP sequence which corresponds to SRSF1 amino acids 132-144, a V5
tag and the protein transduction domain TAT amino acids 47-57:
(SEQ ID NO 90)
Nt-GSWQDLKDHMREAGGGKPIPNPLLGLDSTGGYGRKKRRQRRR-Ct
5/Sequence of the scAAV_SRSF1 89-120 CPP_GFP circular sequence (5,692 bp)
(SEQ ID NO 74)
5′...
GCTCTTCCGCTTCCTCGCTCACTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCGCCCCCCTGACGAG
CATCACAAAAATCGACGCTCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGGACAGTATTTGGTA
TCTGCGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCCCAGTGCTGCAA
TGATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTGGTGTCACGCTCGTCGTT
TGGTATGGCTTCATTCAGCTCCGGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAGGATOTT
ACCGCTGTTGAGATCCAGTTCGATGTAACCCACTCGTGCACCCAACTGATCTTCAGCATCTTTTACTTTCACC
AGCGTTTCTGGGTGAGCAAAAACAGGAAGGCAAAATGCCGCAAAAAAGGGAATAAGGGCGACACGGAAATG
TTGAATACTCATACTCTTCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCCACCTGACGT
CTAAGAAACCATTATTATCATGACATTAACCTATAAAAATAGGCGTATCACGAGGCCCTTTCGTCTCGCGCGT
TTCGGTGATGACGGTGAAAACCTCTGACACATGCAGCTCCCGGAGACGGTCACAGCTTGTCTGTAAGCGGA
TGCCGGGAGCAGACAAGCCCGTCAGGGCGCGTCAGCGGGTGTTGGCGGGTGTCGGGGCTGGCTTAACTA
TGCGGCATCAGAGCAGATTGTACTGAGAGTGCACCATATGCGGTGTGAAATACCGCACAGATGCGTAAGGA
GAAAATACCGCATCAGGCGATTCCAACATCCAATAAATCATACAGGCAAGGCAAAGAATTAGCAAAATTAAG
CAATAAAGCCTCAGAGCATAAAGCTAAATCGGTTGTACCAAAAACATTATGACCCTGTAATACTTTTGCGGGA
GAAGCCTTTATTTCAACGCAAGGATAAAAATTTTTAGAACCCTCATATATTTTAAATGCAATGCCTGAGTAATG
TGTAGGTAAAGATTCAAACGGGTGAGAAAGGCCGGAGACAGTCAAATCACCATCAATATGATATTCAACCGT
TCTAGCTGATAAATTCATGCCGGAGAGGGTAGCTATTTTTGAGAGGTCTCTACAAAGGCTATCAGGTCATTG
CCTGAGAGTCTGGAGCAAACAAGAGAATCGCCGGGGGGGGGGGGGGGGGGGCCACTCCCTCTCTGCGCG
CTCGCTCGCTCACTGAGGCCGCCCGGGCAAAGCCCGGGCGTCGGGCGACCTTTGGTCGCCCGGCCTCAG
TGAGCGAGCGAGCGCGCAGAGAGGGAGTGGCCAACTCCATCACTAGGGGTTCCTCAGATCGATCTCTCCC
CAGCATGCGTTTTACCTCCCCAGCATGCCTGCTATTCTCTTCCCAATCCTCCCCCTTGCTGTCCTGCCCCAC
CCCACCCCCCAGAATAGAATGACACCTACTCAGACAATGCGATGCAATTTCCTCATTTTATTAGGAAAGGAC
AGTGGGAGTGGCACCTTCCAGGGTCAAGGAAGGCACGGGGGAGGGGCAAACAACAGATGGCTGGCAACTA
GAAGGCACAGTCGAGGCTGATCAGCGAGCTCTAGGAATTTTACTTGTACAGCTCGTCCATGCCGAGAGTGA
TCCCGGCGGCGGTCACGAACTCCAGCAGGACCATGTGATCGCGCTTCTCGTTGGGGTCTTTGCTCAGGGC
GGACTGGGTGCTCAGGTAGTGGTTGTCGGGCAGCAGCACGGGGCCGTCGCCGATGGGGGTGTTCTGCTG
GTAGTGGTCGGCGAGCTGCACGCTGCCGTCCTCGATGTTGTGGCGGATCTTGAAGTTCACCTTGATGCCGT
TCTTCTGCTTGTCGGCCATGATATAGACGTTGTGGCTGTTGTAGTTGTACTCCAGCTTGTGCCCCAGGATGT
TGCCGTCCTCCTTGAAGTCGATGCCCTTCAGCTCGATGCGGTTCACCAGGGTGTCGCCCTCGAACTTCACC
TCGGCGCGGGTCTTGTAGTTGCCGTCGTCCTTGAAGAAGATGGTGCGCTCCTGGACGTAGCCTTCGGGCAT
GGCGGACTTGAAGAAGTCGTGCTGCTTCATGTGGTCGGGGTAGCGGCTGAAGCACTGCACGCCGTAGGTC
AGGGTGGTCACGAGGGTGGGCCAGGGCACGGGCAGCTTGCCGGTGGTGCAGATGAACTTCAGGGTCAGC
TTGCCGTAGGTGGCATCGCCCTCGCCCTCGCCGGACACGCTGAACTTGTGGCCGTTTACGTCGCCGTCCA
GCTCGACCAGGATGGGCACCACCCCGGTGAACAGCTCCTCGCCCTTGCTCACCATGCCTGTGTTCTGGCG
GCAAACCCGTTGCGAAAAAGAACGTTCACGGCGACTACTGCACTTATATACGGTTCTCCCCCACCCTCGGG
AAAAAGGCGGAGCCAGTACACGACATCACTTTCCCAGTTTACCCCGCGCCACCTTCTCTAGGCACCGGATC
AATTGCCGACCCCTCCCCCCAACTTCTCGGGGACTGTGGGCGATGTGCGCTCTGCCCACTGAATCTTCTCG
AGCCTCTAGATACCACAGGCTGCGCAACTGTTGGGAAGGGCGATCGGTGCGGGCCTCTTCGCTATTACGCC
AGCTGGCGAAAGGGGGATGTGCTGCAAGGCGATTAAGTTGGGTAACGCCAGGGTTTTCCCAGTCACGACG
TTGTAAAACGACGGCCAGTGAATTCATATTTGCATGTCGCTATGTGTTCTGGGAAATCACCATAAACGTGAAA
TGTCTTTGGATTTGGGAATCTTATAAGTTCTGTATGAGACCACTCGGATCCGAAGCCACCATGCCGCGCAGC
GGCCGCGGCACCGGCCGCGGTGGGGGGGGCGGTGGAGGTGGCGGAGCCCCGAGAGGCCGCTATGGACC
GCCCAGCCGCCGGAGCGAAGGCGGTGGGAAACCGATTCCCAACCCGCTGCTGGGCCTCGATAGCACCGG
CGGATATGGTCGCAAAAAGCGCAGACAGCGCCGGAGGTAATTTTTTAAGCTTGGCGTAATCATGGTCATAG
CTGTTTCCTGTGTGAAATTGTTATCCGCTCACAATTCCACACAACATACGAGCCGGAAGCATAAAGTGTATCT
AGAGCGGTACCACGCGTGAATTGAATTCAGATCCACGCGTGAATTCCACTCCCTCTCTGCGCGCTCGCTCG
CTCACTGAGGCCGGGCGACCAAAGGTCGCCCGACGCCCGGGCTTTGCCCGGGGGGCCTCAGTGAGCGAG
CGAGCGCGCAGGGCGATGAACGGTAATCGTAAAACTAGCATGTCAATCATATGTACCCCGGTTGATAATCA
GAAAAGCCCCAAAAACAGGAAGATTGTATAAGCAAATATTTAAATTGTAAGCGTTAATATTTTGTTAAAATTCG
CGTTAAATTTTTGTTAAATCAGCTCATTTTTTAACCAATAGGCCGAAATCGGCAAAATCCCTTATAAATCAAAA
GAATAGACCGAGATAGGGTTGAGTGTTGTTCCAGTTTGGAACAAGAGTCCACTATTAAAGAACGTGGACTCC
AACGTCAAAGGGCGAAAAACCGTCTATCAGGGCGATGGCCCACTACGTGAACCATCACCCTAATCAAGTTTT
TTGGGGTCGAGGTGCCGTAAAGCACTAAATCGGAACCCTAAAGGGAGCCCCCGATTTAGAGCTTGACGGG
GAAAGCCGGCGAACGTGGCGAGAAAGGAAGGGAAGAAAGCGAAAGGAGCGGGCGCTAGGGCGCTGGCAA
GTGTAGCGGTCACGCTGCGCGTAACCACCACACCCGCCGCGCTTAATGCGCCGCTACAGGGCGCGTACTA
TGGTTGCTTTGACGAGCACGTATAACGTGCTTTCCTCGTTAGAATCAGAGCGGGAGCTAAACAGGAGGCCG
ATTAAAGGGATTTTAGACAGGAACGGTACGCCAGAATCCTGAGAAGTGTTTTTATAATCAGTGAGGCCACCG
AGTAAAAGAGTCTGTCCATCACGCAAATTAACCGTTGTCGCAATACTTCTTTGATTAGTAATAACATCACTTG
CCTGAGTAGAAGAACTCAAACTATCGGCCTTGCTGGTAATATCCAGAACAATATTACCGCCAGCCATTGCAA
CGGAATCGCCATTCGCCATTCAGGCTGCGCAACTGTTGGGAAGGGCGATCGGTGCGGGCCTCTTCGCTATT
ACGCCAGCTGCATTAATGAATCGGCCAACGCGCGGGGAGAGGCGGTTTGCGTATTGGGC-3′
6/5/Sequence of the scAAV_SRSF1 132-144 CPP_GFP circular sequence (5,651 bp)
(SEQ ID NO 1)
5′...
GCTCTTCCGCTTCCTCGCTCACTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCGCCCCCCTGACGAG
CATCACAAAAATCGACGCTCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGGACAGTATTTGGTA
TCTGCGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCCCAGTGCTGCAA
TGATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTGGTGTCACGCTCGTCGTT
TGGTATGGCTTCATTCAGCTCCGGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAGGATCTT
ACCGCTGTTGAGATCCAGTTCGATGTAACCCACTCGTGCACCCAACTGATCTTCAGCATCTTTTACTTTCACC
AGCGTTTCTGGGTGAGCAAAAACAGGAAGGCAAAATGCCGCAAAAAAGGGAATAAGGGCGACACGGAAATG
TTGAATACTCATACTCTTCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCCACCTGACGT
CTAAGAAACCATTATTATCATGACATTAACCTATAAAAATAGGCGTATCACGAGGCCCTTTCGTCTCGCGCGT
TTCGGTGATGACGGTGAAAACCTCTGACACATGCAGCTCCCGGAGACGGTCACAGCTTGTCTGTAAGCGGA
TGCCGGGAGCAGACAAGCCCGTCAGGGCGCGTCAGCGGGTGTTGGCGGGTGTCGGGGCTGGCTTAACTA
TGCGGCATCAGAGCAGATTGTACTGAGAGTGCACCATATGCGGTGTGAAATACCGCACAGATGCGTAAGGA
GAAAATACCGCATCAGGCGATTCCAACATCCAATAAATCATACAGGCAAGGCAAAGAATTAGCAAAATTAAG
CAATAAAGCCTCAGAGCATAAAGCTAAATCGGTTGTACCAAAAACATTATGACCCTGTAATACTTTTGCGGGA
GAAGCCTTTATTTCAACGCAAGGATAAAAATTTTTAGAACCCTCATATATTTTAAATGCAATGCCTGAGTAATG
TGTAGGTAAAGATTCAAACGGGTGAGAAAGGCCGGAGACAGTCAAATCACCATCAATATGATATTCAACCGT
TCTAGCTGATAAATTCATGCCGGAGAGGGTAGCTATTTTTGAGAGGTCTCTACAAAGGCTATCAGGTCATTG
CCTGAGAGTCTGGAGCAAACAAGAGAATCGCCGGGGGGGGGGGGGGGGGGGCCACTCCCTCTCTGCGCG
CTCGCTCGCTCACTGAGGCCGCCCGGGCAAAGCCCGGGCGTCGGGCGACCTTTGGTCGCCCGGCCTCAG
TGAGCGAGCGAGCGCGCAGAGAGGGAGTGGCCAACTCCATCACTAGGGGTTCCTCAGATCGATCTCTCCC
CAGCATGCGTTTTACCTCCCCAGCATGCCTGCTATTCTCTTCCCAATCCTCCCCCTTGCTGTCCTGCCCCAC
CCCACCCCCCAGAATAGAATGACACCTACTCAGACAATGCGATGCAATTTCCTCATTTTATTAGGAAAGGAC
AGTGGGAGTGGCACCTTCCAGGGTCAAGGAAGGCACGGGGGAGGGGCAAACAACAGATGGCTGGCAACTA
GAAGGCACAGTCGAGGCTGATCAGCGAGCTCTAGGAATTTTACTTGTACAGCTCGTCCATGCCGAGAGTGA
TCCCGGCGGCGGTCACGAACTCCAGCAGGACCATGTGATCGCGCTTCTCGTTGGGGTCTTTGCTCAGGGC
GGACTGGGTGCTCAGGTAGTGGTTGTCGGGCAGCAGCACGGGGCCGTCGCCGATGGGGGTGTTCTGCTG
GTAGTGGTCGGCGAGCTGCACGCTGCCGTCCTCGATGTTGTGGCGGATCTTGAAGTTCACCTTGATGCCGT
TCTTCTGCTTGTCGGCCATGATATAGACGTTGTGGCTGTTGTAGTTGTACTCCAGCTTGTGCCCCAGGATGT
TGCCGTCCTCCTTGAAGTCGATGCCCTTCAGCTCGATGCGGTTCACCAGGGTGTCGCCCTCGAACTTCACC
TCGGCGCGGGTCTTGTAGTTGCCGTCGTCCTTGAAGAAGATGGTGCGCTCCTGGACGTAGCCTTCGGGCAT
GGCGGACTTGAAGAAGTCGTGCTGCTTCATGTGGTCGGGGTAGCGGCTGAAGCACTGCACGCCGTAGGTC
AGGGTGGTCACGAGGGTGGGCCAGGGCACGGGCAGCTTGCCGGTGGTGCAGATGAACTTCAGGGTCAGC
TTGCCGTAGGTGGCATCGCCCTCGCCCTCGCCGGACACGCTGAACTTGTGGCCGTTTACGTCGCCGTCCA
GCTCGACCAGGATGGGCACCACCCCGGTGAACAGCTCCTCGCCCTTGCTCACCATGCCTGTGTTCTGGCG
GCAAACCCGTTGCGAAAAAGAACGTTCACGGCGACTACTGCACTTATATACGGTTCTCCCCCACCCTCGGG
AAAAAGGCGGAGCCAGTACACGACATCACTTTCCCAGTTTACCCCGCGCCACCTTCTCTAGGCACCGGATC
AATTGCCGACCCCTCCCCCCAACTTCTCGGGGACTGTGGGCGATGTGCGCTCTGCCCACTGAATCTTCTCG
AGCCTCTAGATACCACAGGCTGCGCAACTGTTGGGAAGGGCGATCGGTGCGGGCCTCTTCGCTATTACGCC
AGCTGGCGAAAGGGGGATGTGCTGCAAGGCGATTAAGTTGGGTAACGCCAGGGTTTTCCCAGTCACGACG
TTGTAAAACGACGGCCAGTGAATTCATATTTGCATGTCGCTATGTGTTCTGGGAAATCACCATAAACGTGAAA
TGTCTTTGGATTTGGGAATCTTATAAGTTCTGTATGAGACCACTCGGATCCGTTTAGTGAACCGTCAGAAGCC
ACCATGGGCAGCTGGCAGGATCTGAAAGATCATATGCGCGAAGCCGGCGGTGGGAAACCGATTCCCAACC
CGCTGCTGGGCCTCGATAGCACCGGCGGATATGGTCGCAAAAAGCGCAGACAGCGCCGGAGGTAATTTTTT
AAGCTTGGCGTAATCATGGTCATAGCTGTTTCCTGTGTGAAATTGTTATCCGCTCACAATTCCACACAACATA
CGAGCCGGAAGCATAAAGTGTATCTAGAGCGGTACCACGCGTGAATTGAATTCAGATCCACGCGTGAATTC
CACTCCCTCTCTGCGCGCTCGCTCGCTCACTGAGGCCGGGCGACCAAAGGTCGCCCGACGCCCGGGCTTT
GCCCGGGCGGCCTCAGTGAGCGAGCGAGCGCGCAGGGCGATGAACGGTAATCGTAAAACTAGCATGTCAA
TCATATGTACCCCGGTTGATAATCAGAAAAGCCCCAAAAACAGGAAGATTGTATAAGCAAATATTTAAATTGT
AAGCGTTAATATTTTGTTAAAATTCGCGTTAAATTTTTGTTAAATCAGCTCATTTTTTAACCAATAGGCCGAAAT
CGGCAAAATCCCTTATAAATCAAAAGAATAGACCGAGATAGGGTTGAGTGTTGTTCCAGTTTGGAACAAGAG
TCCACTATTAAAGAACGTGGACTCCAACGTCAAAGGGCGAAAAACCGTCTATCAGGGCGATGGCCCACTAC
GTGAACCATCACCCTAATCAAGTTTTTTGGGGTCGAGGTGCCGTAAAGCACTAAATCGGAACCCTAAAGGGA
GCCCCCGATTTAGAGCTTGACGGGGAAAGCCGGCGAACGTGGCGAGAAAGGAAGGGAAGAAAGCGAAAG
GAGCGGGCGCTAGGGCGCTGGCAAGTGTAGCGGTCACGCTGCGCGTAACCACCACACCCGCCGCGCTTA
ATGCGCCGCTACAGGGCGCGTACTATGGTTGCTTTGACGAGCACGTATAACGTGCTTTCCTCGTTAGAATCA
GAGCGGGAGCTAAACAGGAGGCCGATTAAAGGGATTTTAGACAGGAACGGTACGCCAGAATCCTGAGAAGT
GTTTTTATAATCAGTGAGGCCACCGAGTAAAAGAGTCTGTCCATCACGCAAATTAACCGTTGTCGCAATACTT
CTTTGATTAGTAATAACATCACTTGCCTGAGTAGAAGAACTCAAACTATCGGCCTTGCTGGTAATATCCAGAA
CAATATTACCGCCAGCCATTGCAACGGAATCGCCATTCGCCATTCAGGCTGCGCAACTGTTGGGAAGGGCG
ATCGGTGCGGGCCTCTTCGCTATTACGCCAGCTGCATTAATGAATCGGCCAACGCGCGGGGAGAGGCGGT
TTGCGTATTGGGC-3′

Functionality of scAAV9-Driven Expression of SRSF1-shRNA10 in Mouse Brains.

C9ORF72-ALS/FTD mice were injected via cisterna magna at post-natal day 1 (P1) with either 8×10¹⁰ scAAV9_Ctrl-shRNA_GFP vector genomes (vg) or 6×10¹⁰ scAAV9_SRSF1-shRNA10_GFP vg. Animals were sacrificed 1 month and 3 months post injections. Western blots show that the scAAV9_SRSF1-shRNA10_GFP virus leads to specific depletion of SRSF1 in C9ORF72-ALS/FTD mice as well as in wild type C57B16 mice (not shown) while the Ctrl-shRNA has no effect. GAPDH is used as a loading control.

### Example 1

### 2/ iMN Imaging Examples at Day 24

High content automated imaging (Opera Phenix) was used to quantify surving MNs at Day 1, 2 and 3 of imaging. Images (FIG. 2) show that MNs treated with lentivirus expressing SRSF1-miRNA retain processes/axons characteristic of neurons compared to MN treated with LV_Ctrl-RNAi which generate and die.

### 3/ iMN Quantification

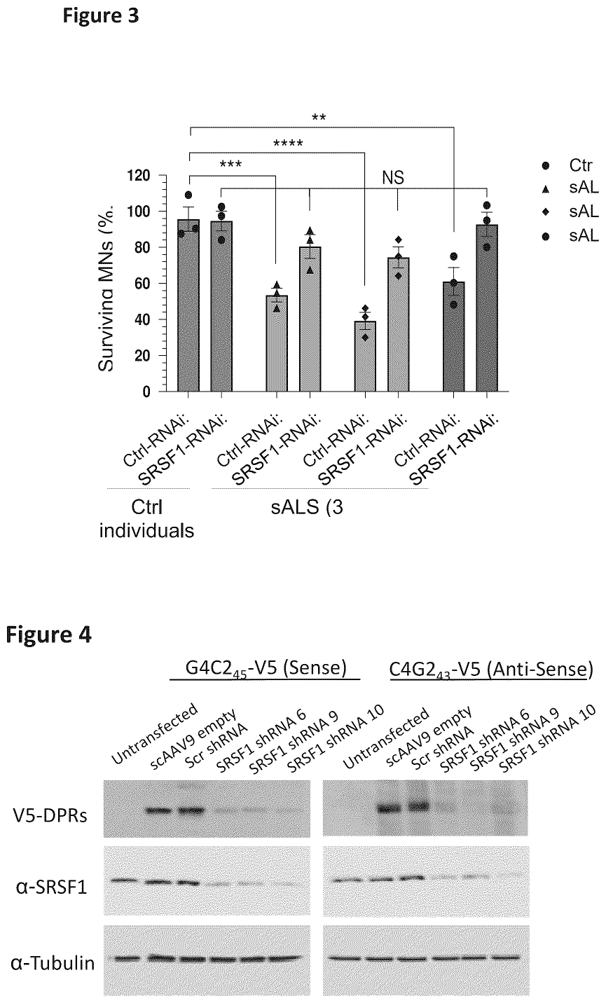

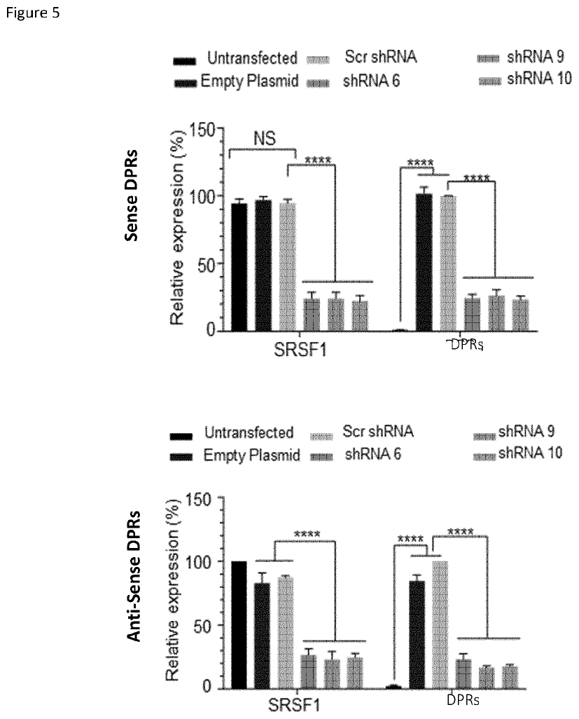

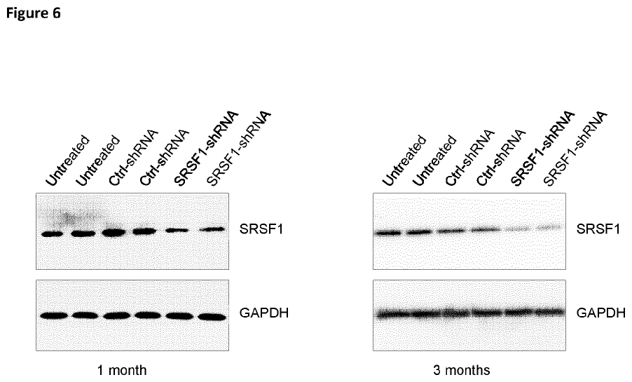

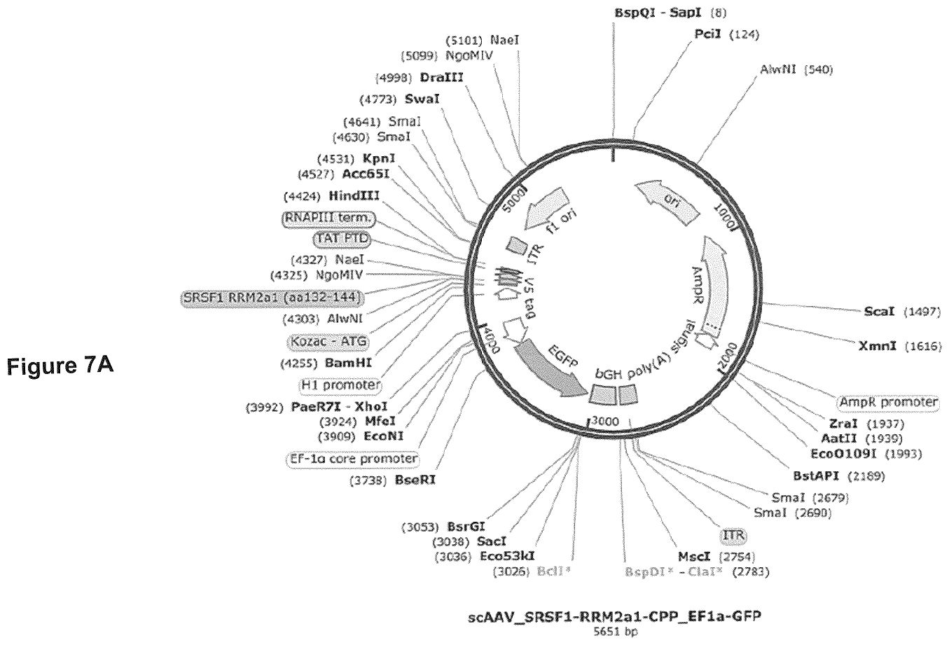

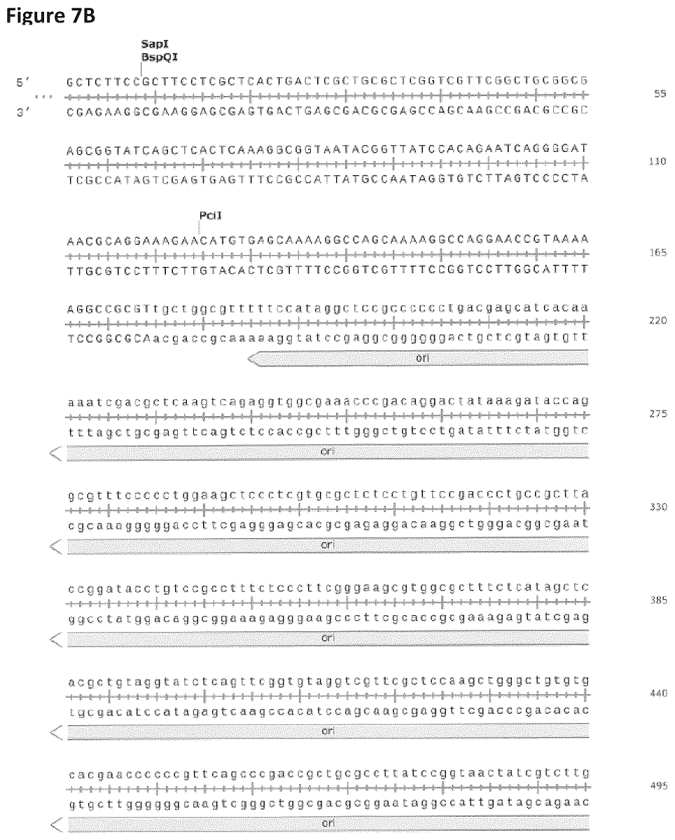

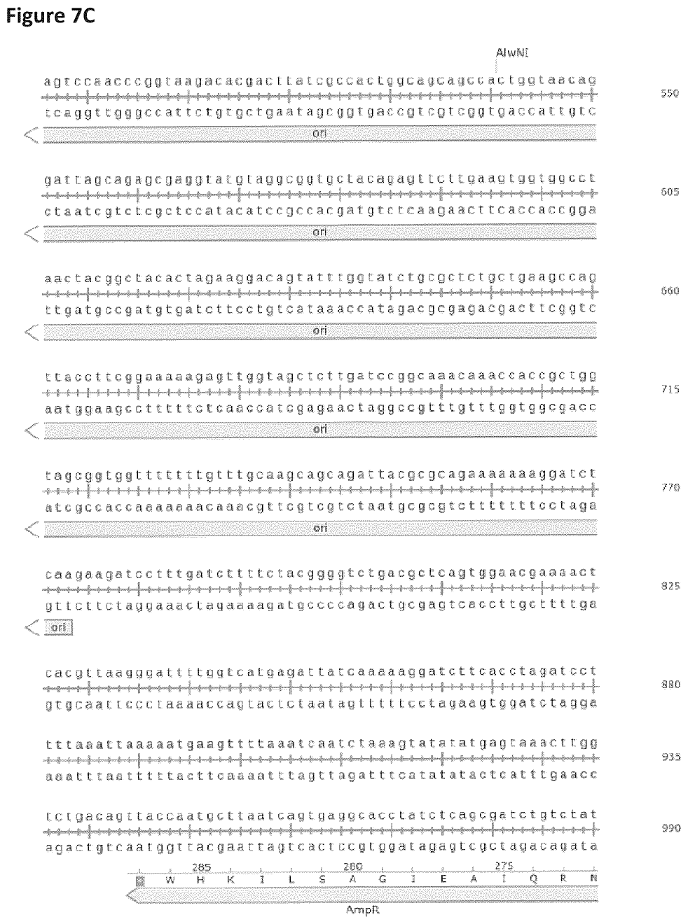

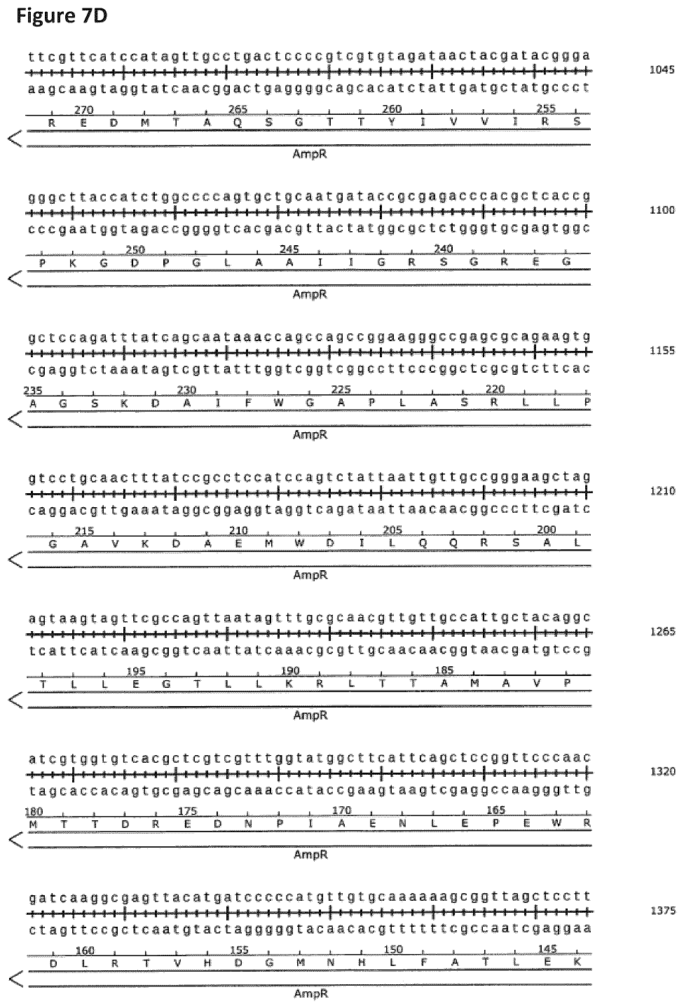

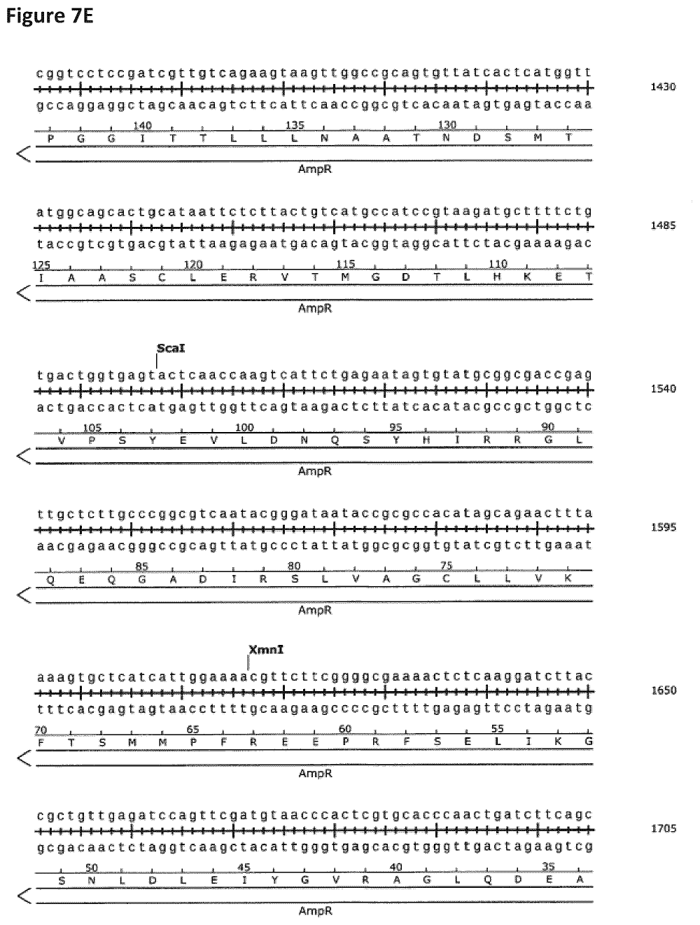

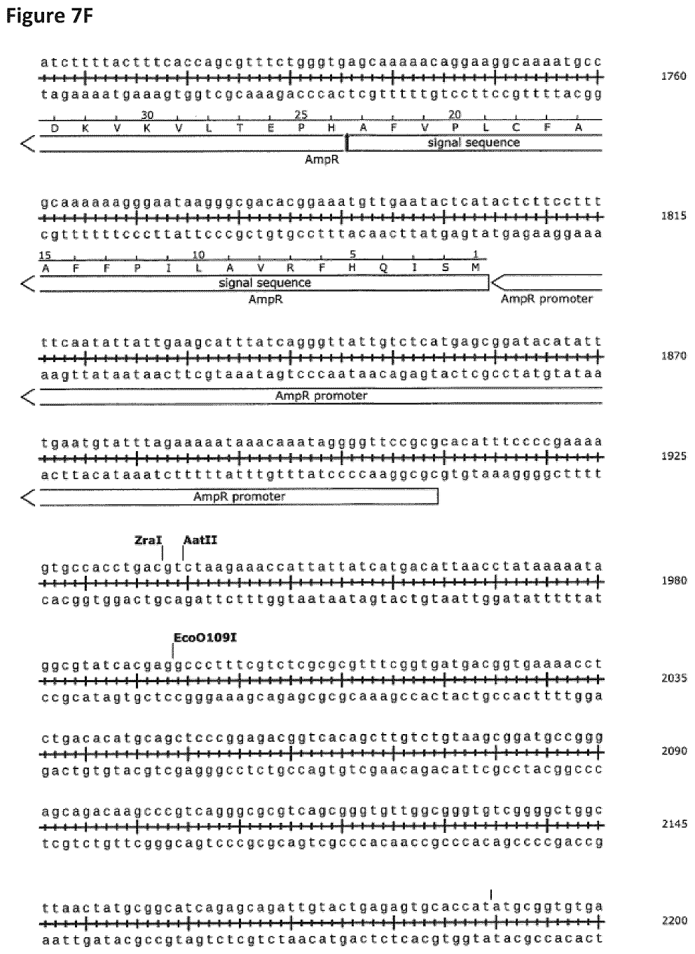

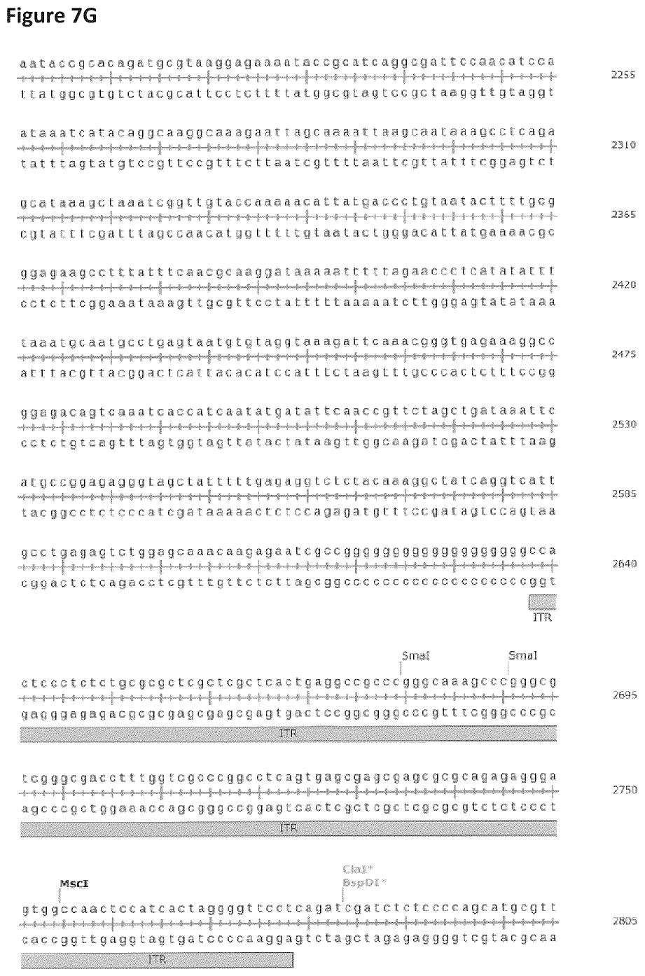

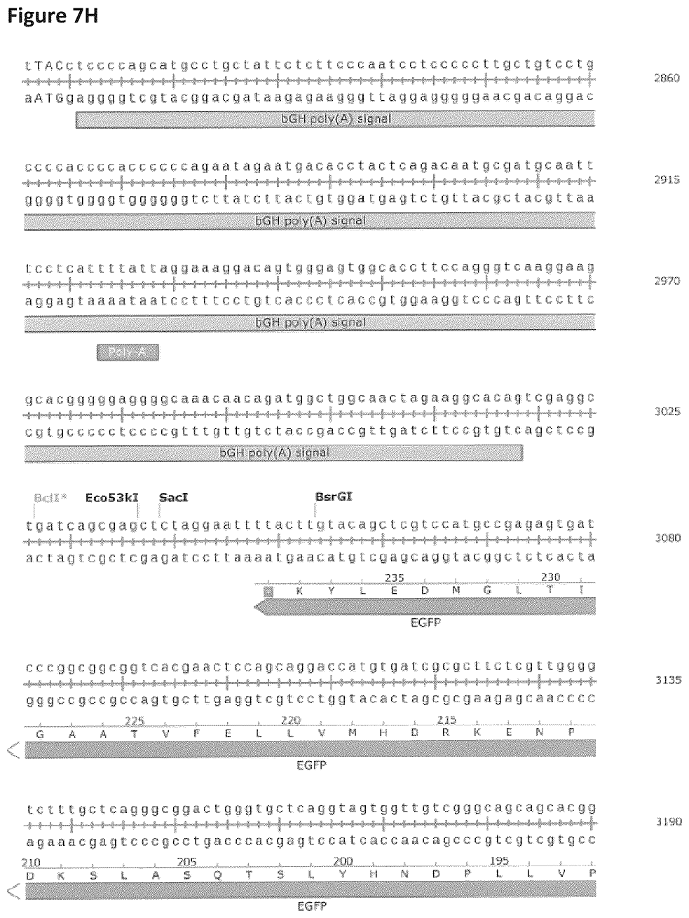

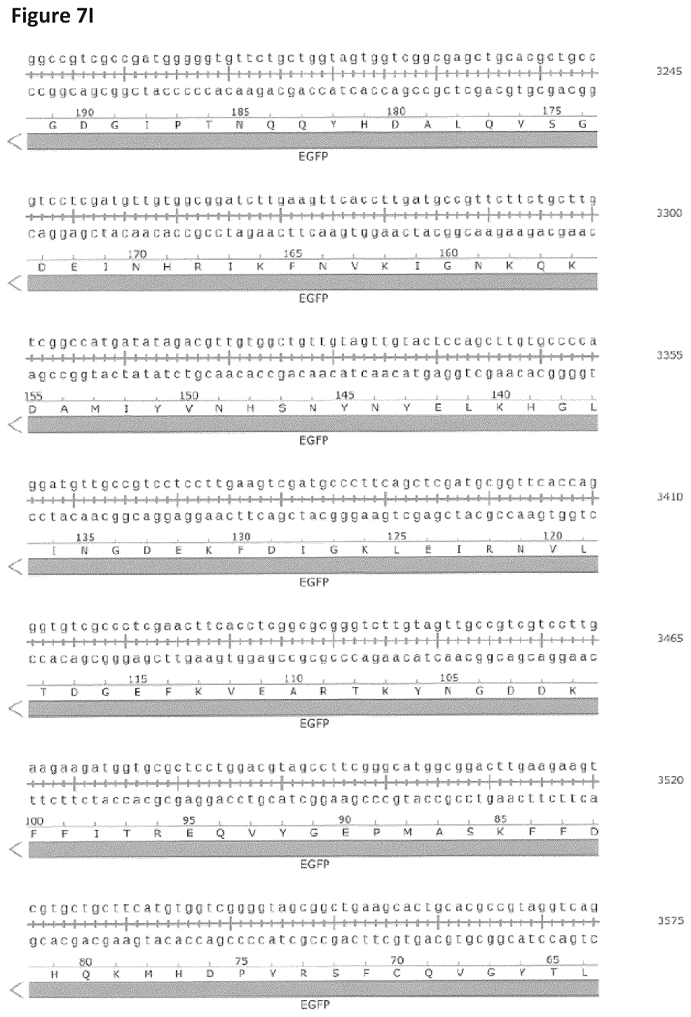

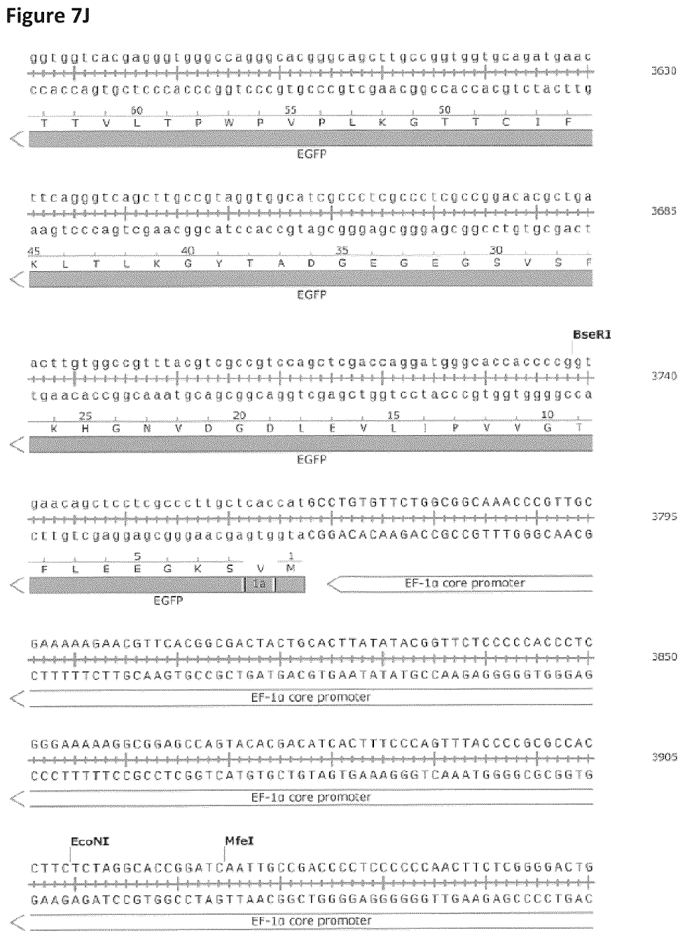

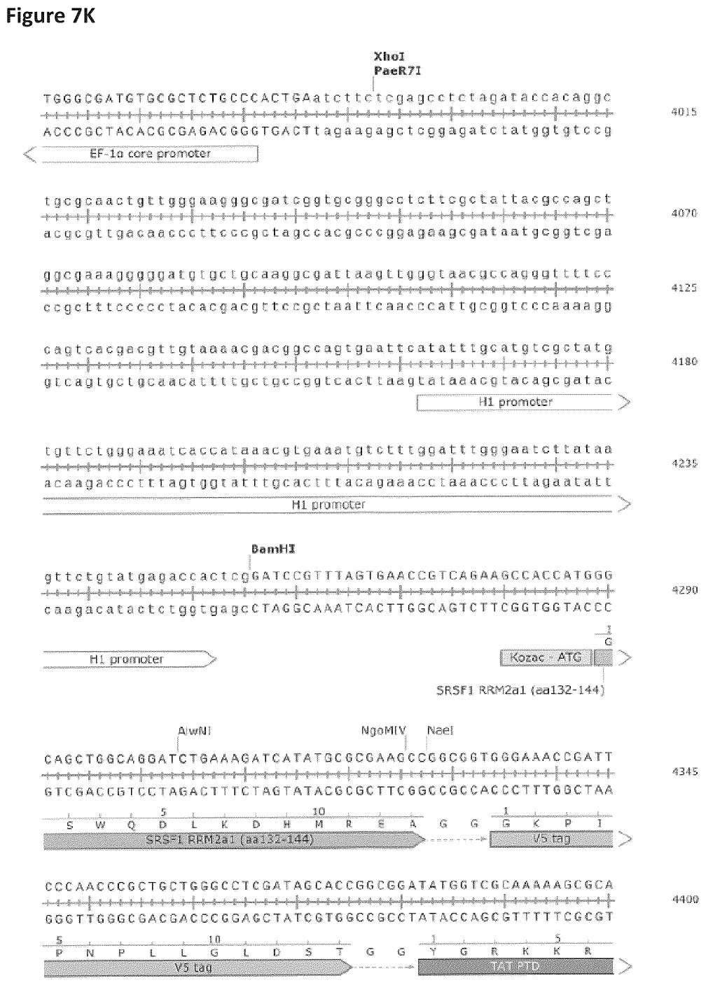

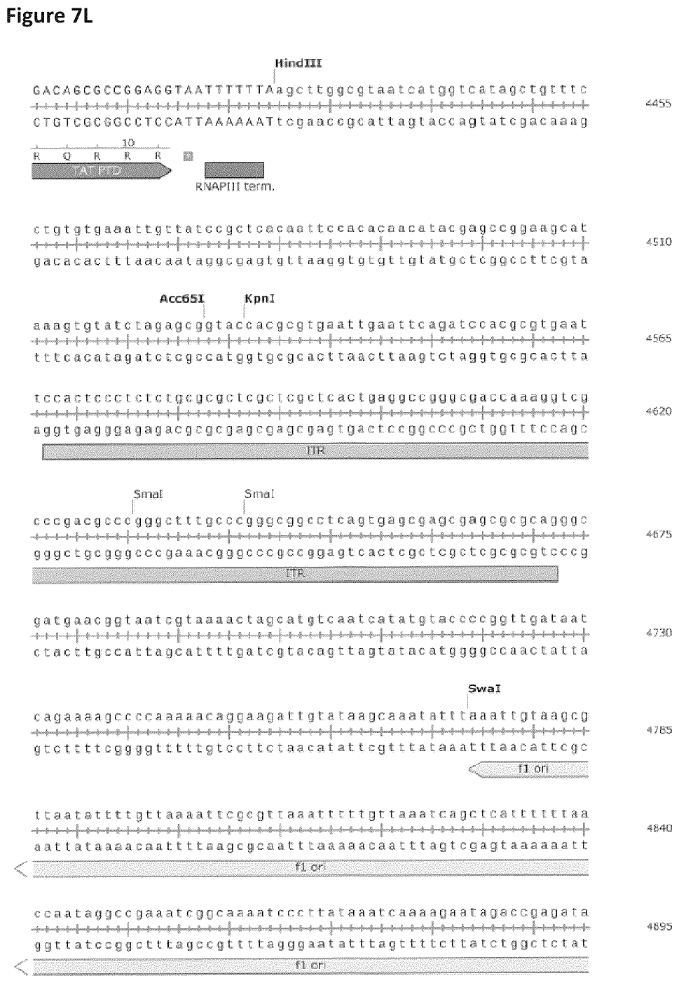

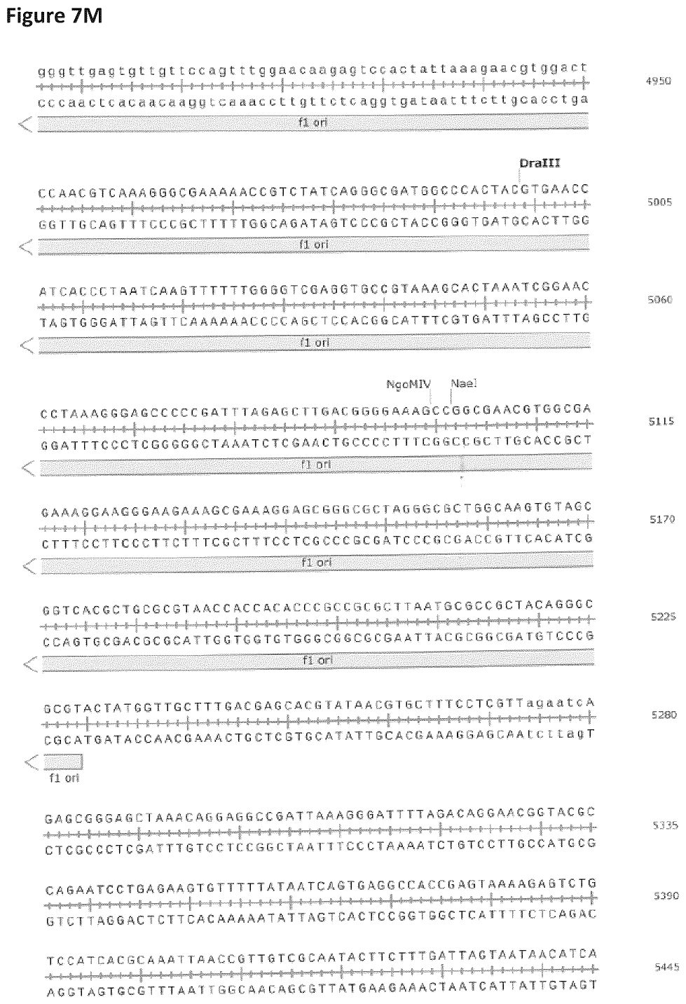

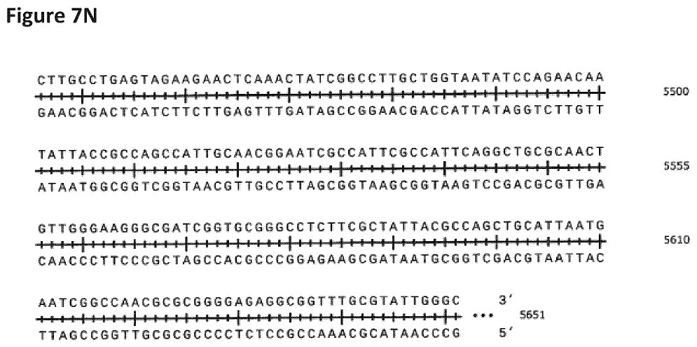

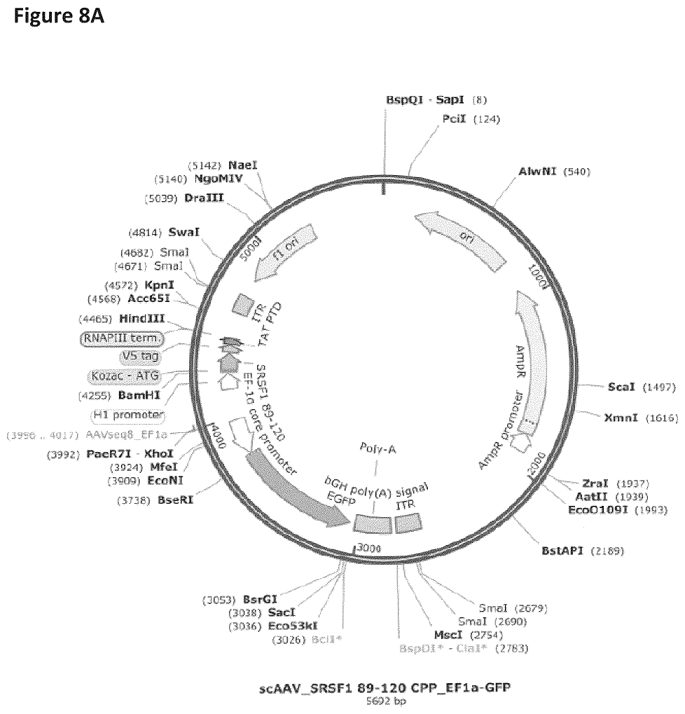

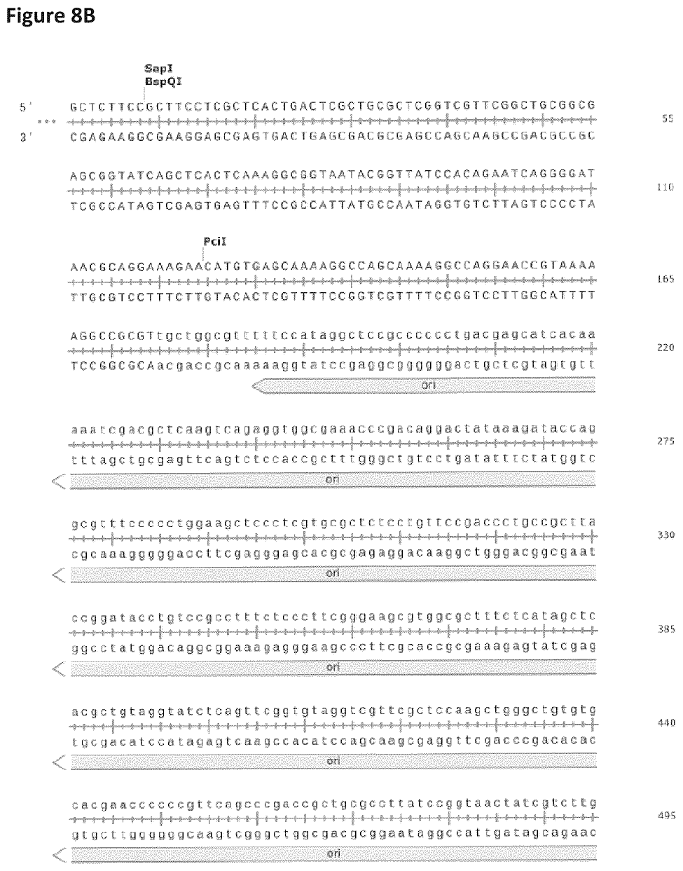

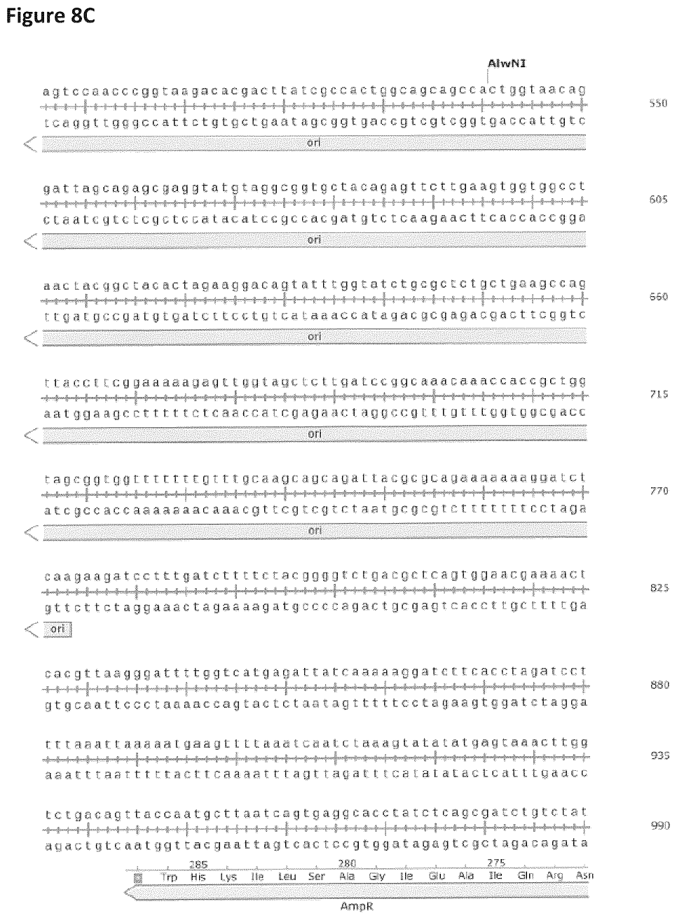

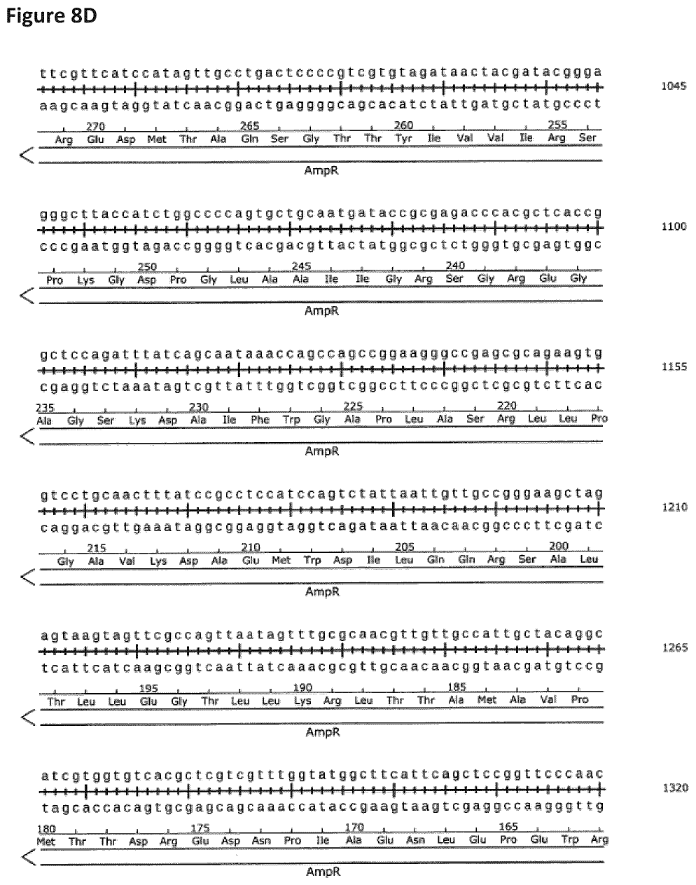

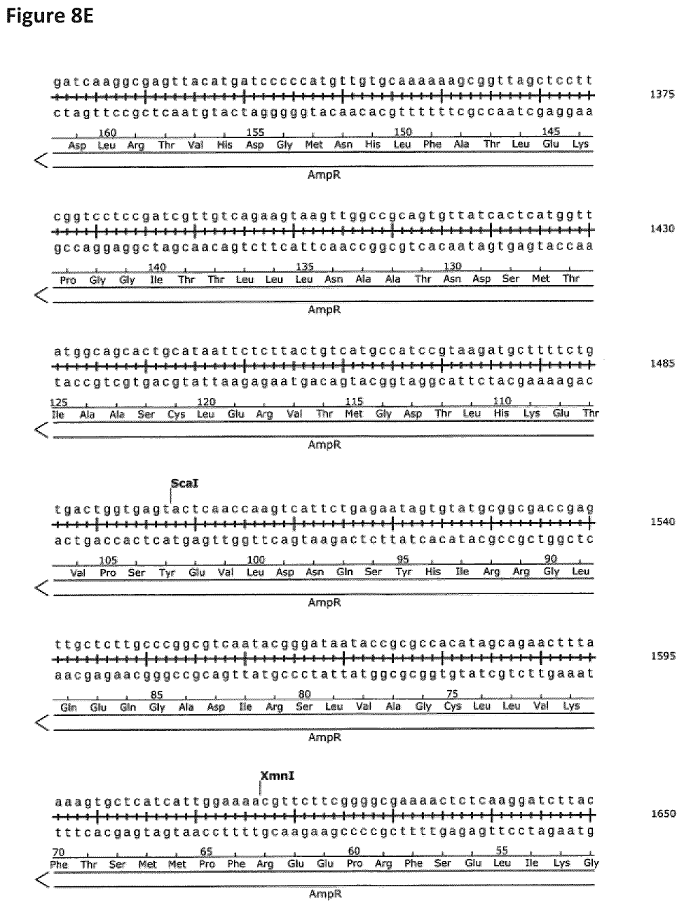

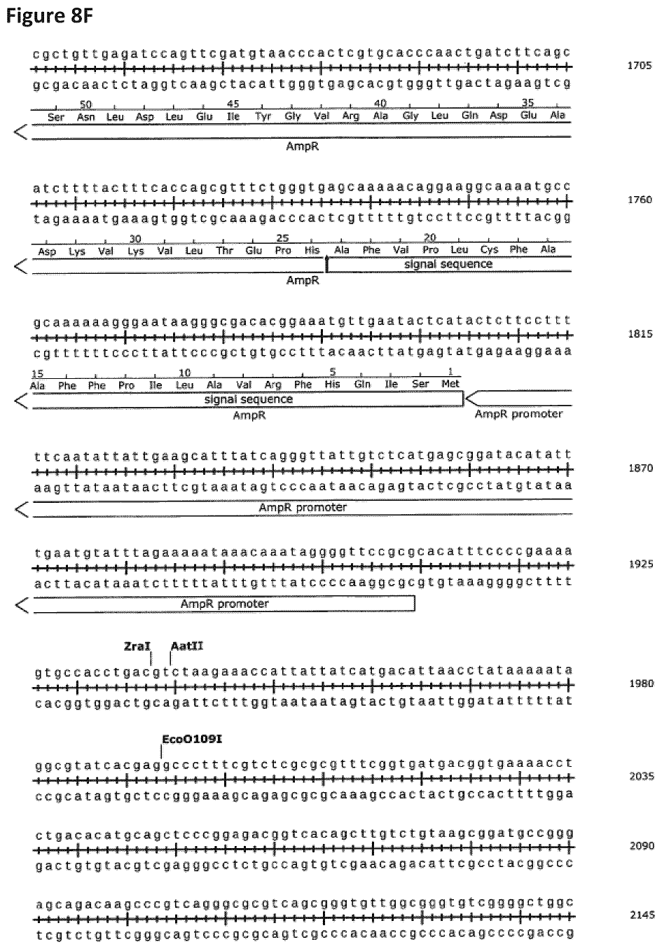

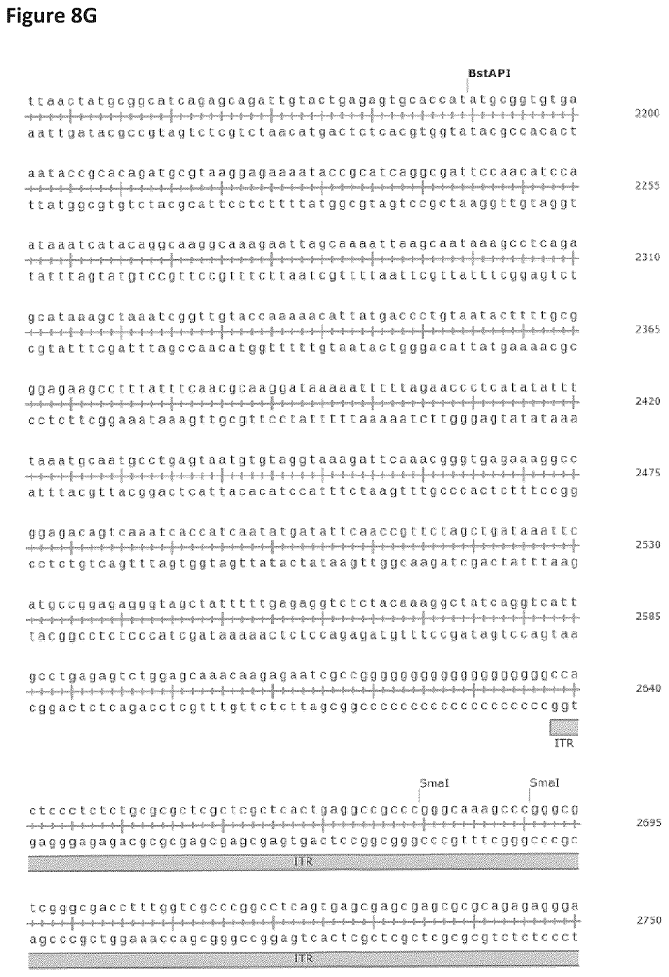

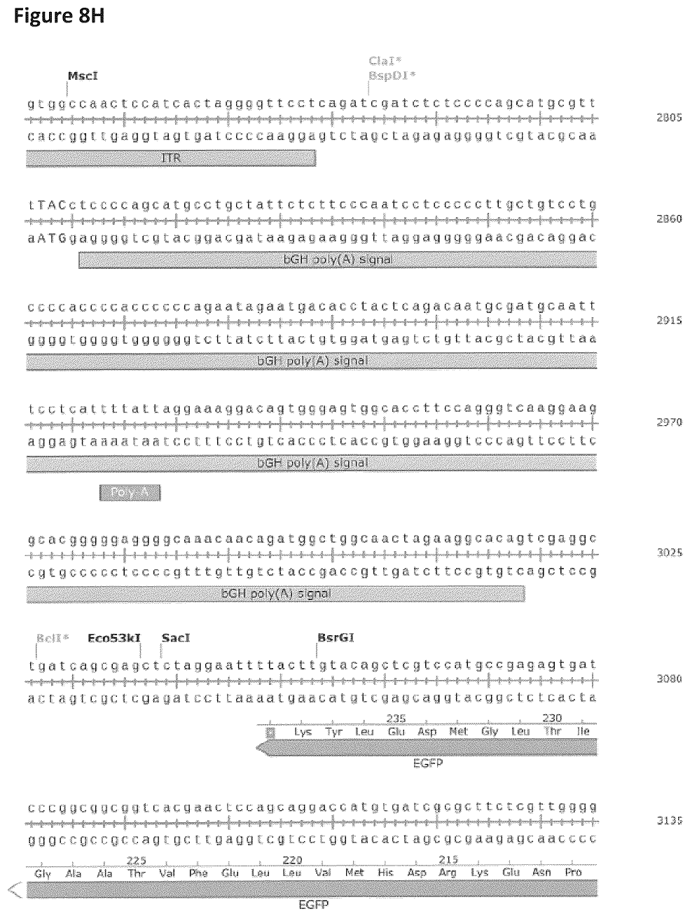

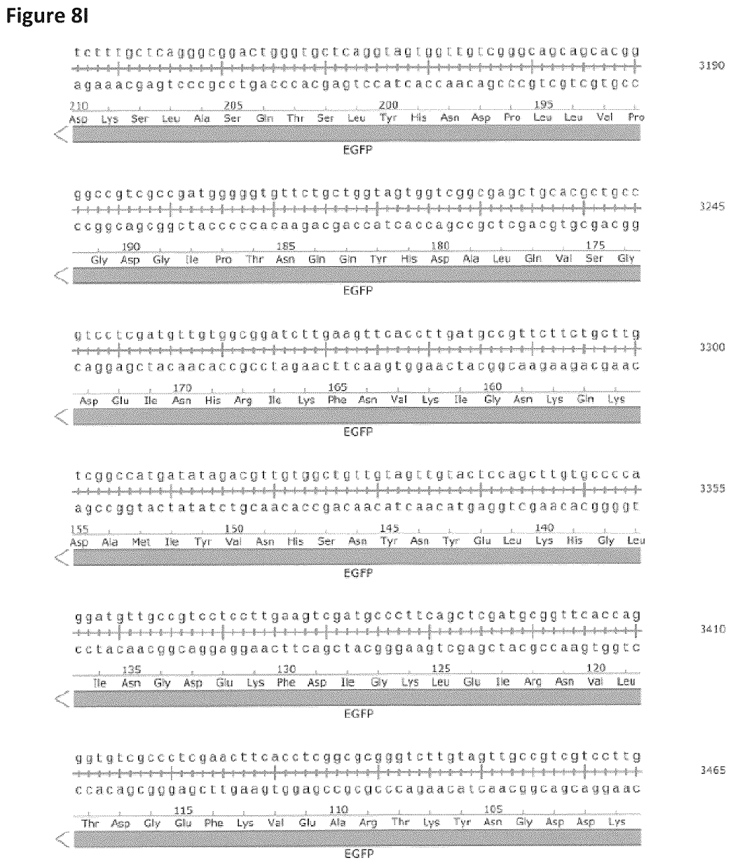

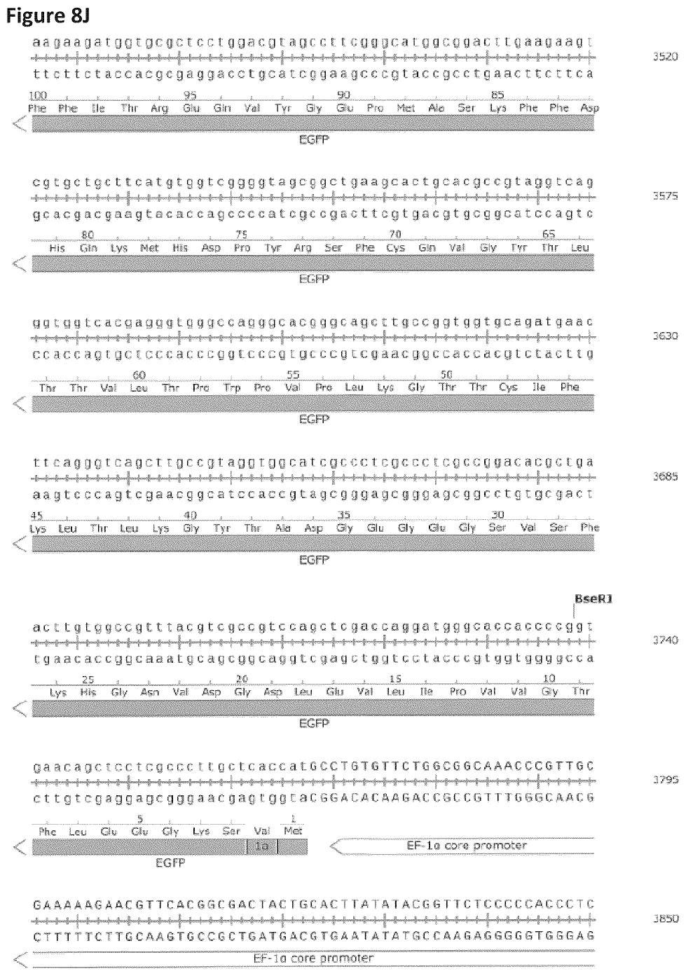

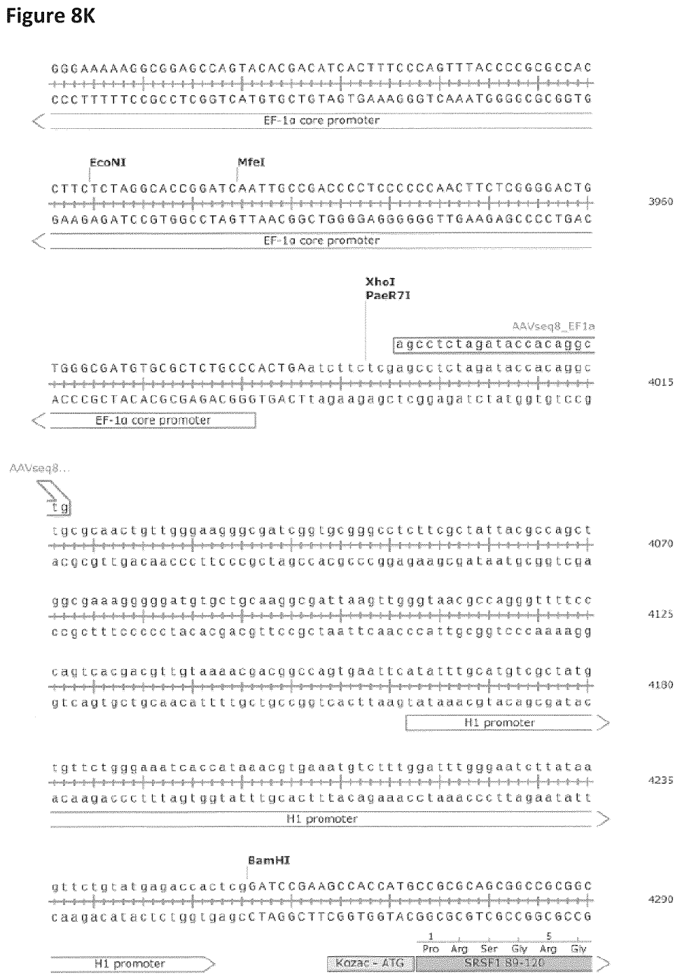

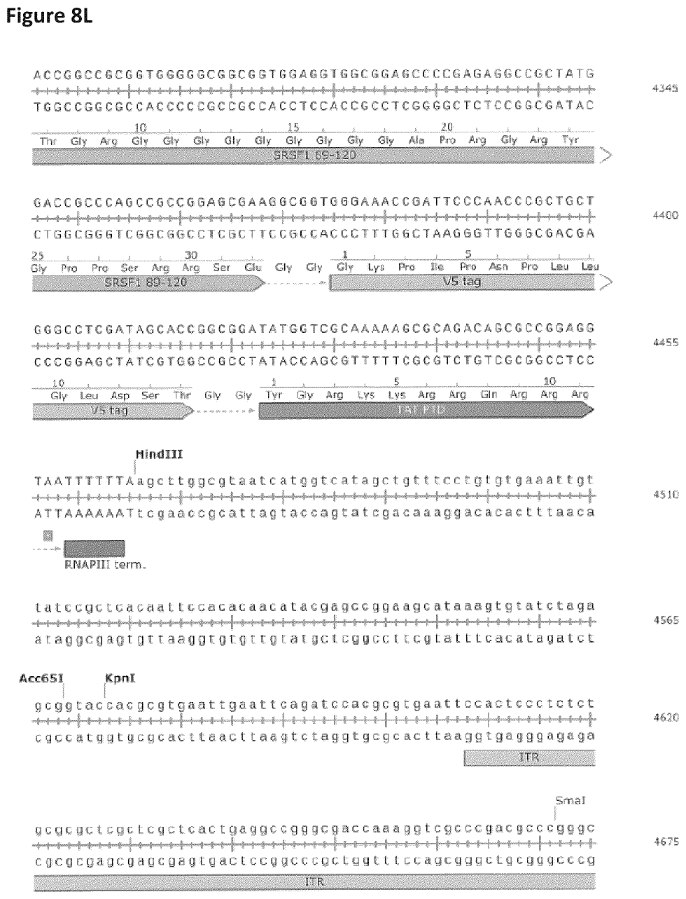

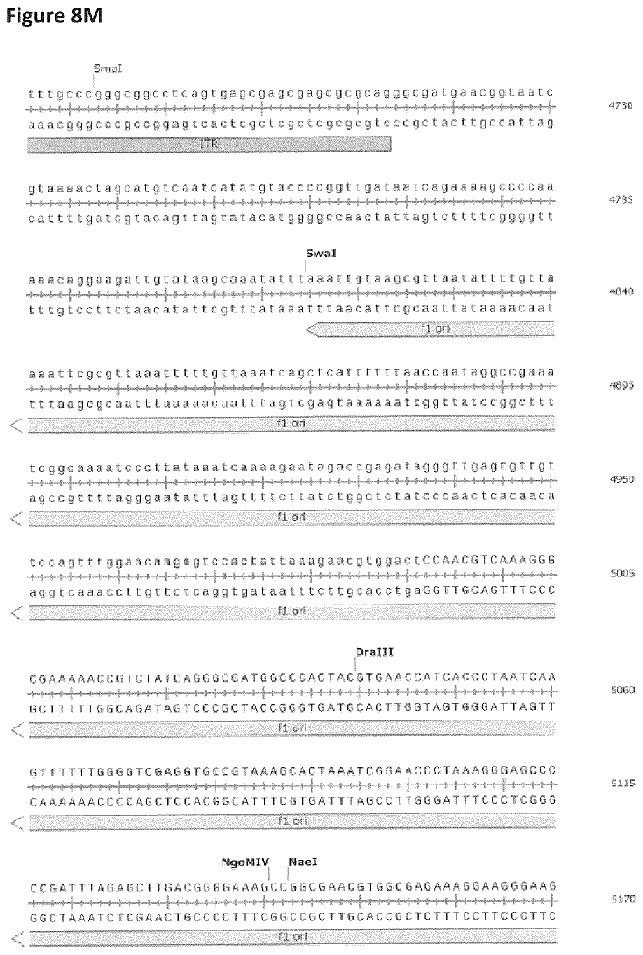

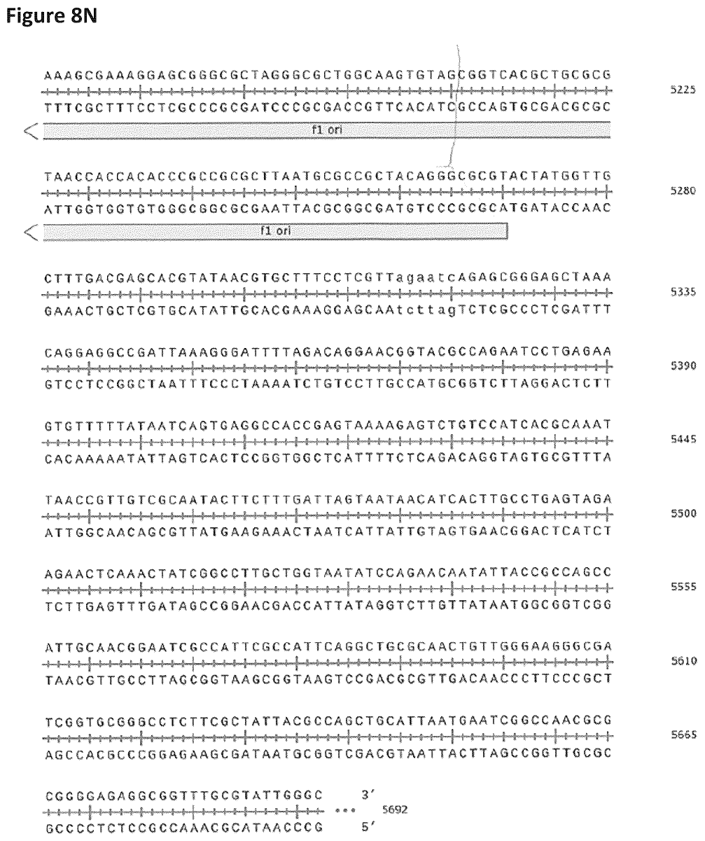

Co-culture of healthy control and sALS patient-derived MN and astrocytes show that LV_SRSF1-RNAi specifically promotes sALS MN survival in levels comparable to the depletion of SRSF1 in C9ORF72-ALD patient-derived MNs (Hautbergue G M et al, Nature Communications 2017; 8:16063; Castelli et al. bioRxiv 2021.05.23.445325v2) Bar charts show MN survival expressed as a ratio of MNs quantified at counting day 3 over day 1 (%). 2-way ANOVA with Tukey's multiple comparison test; NS: non-significant; **: p<0.01; ***: p<0.001; ****: p<0.0001 (FIG. 3)

### Example 2

### 4/ Testing the Functionality of SRSF1-shRNAs in Human Cells and Mouse Brains

scAAV plasmids co-expressing GFP and SRSF1 shRNA 6, 9 or 10 were co-transfected with either sense or antisense C9ORF72-repeat reporter constructs expressing V5-tagged sense or antisense dipeptide repeat proteins (DPRs) in all frames in a repeat-associated non-AUG (RAN) translation manner. Western immunoblotting shows that all 3 shRNAs lead to efficient depletion of SRSF1 and inhibition of the RAN translation of V5-tagged DPRs. SRSF1-shRNA10 was selected for viral production and further experiments in mice as it has the lowest POTS score and predicted genome-wide off-target effect in both mouse and human.

### Example 3

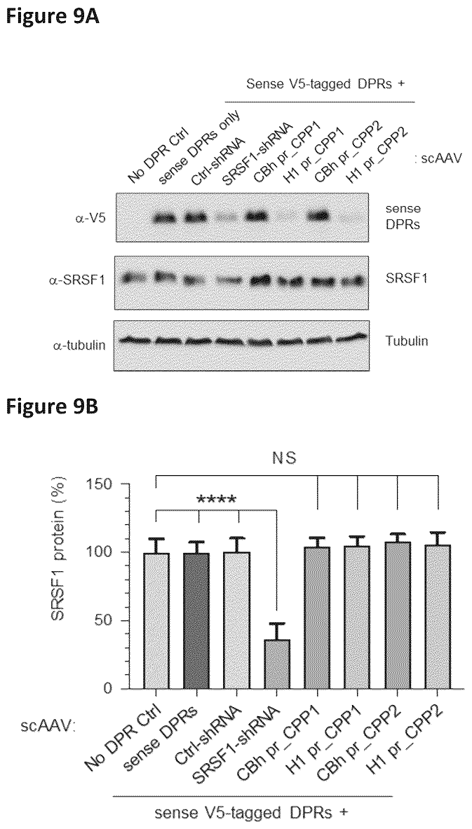

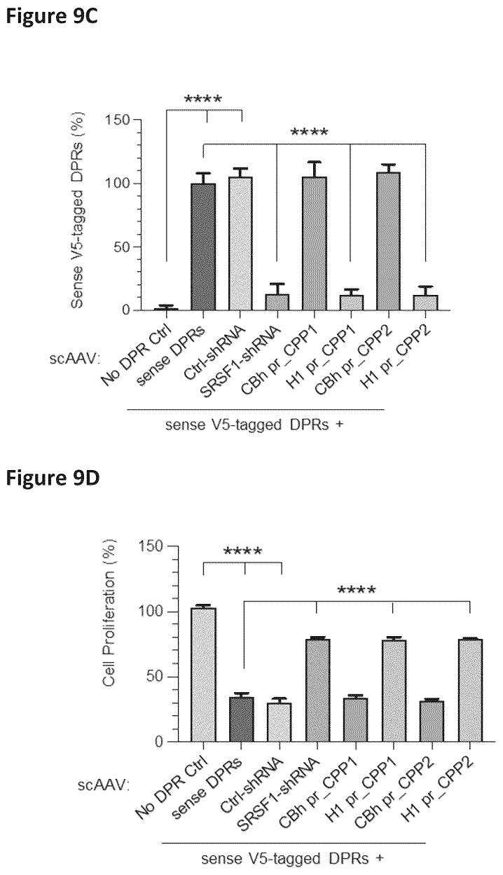

scAAV SRSF1-shRNA, CPP1 and CPP2 inhibits the production of sense DPRs and rescue the DPR-associated cytotoxicity in a human cell model of C9ORF72-ALS/FTD. Human HEK293T cells were co-transfected with sense G4C2×45 C9ORF72-repeat plasmid expressing sense V5-tagged dipeptide-repeat protein (DPRs) in a RAN translation manner and scAAV plasmids expressing SRSF1-shRNA10 or 2 different cell permeable peptides (CPP1: SRSF1 aa89-120 CPP (SEQ ID NO 59) and CPP2: SRSF1 aa132-144 CPP (SEQ ID NO 75)). We tested potential expression under mammalian ubiquitous RNA polymerase II (CBh) or RNA polymerase Ill (H1) promoters. As shown in FIG. 9A Western blots show depletion of SRSF1 and inhibition of the RAN translation of sense DPRs upon co-transfection with scAAV SRSF1-shRNA10_GFP, H1-CPP1_GFP and H1-CPP2_GFP, but not when CPPs transcription is driven the RNAPII promoter. SRSF1 and DPRs expression levels are quantified in triplicate biological experiments in FIGS. 9B and 9C respectively (Bar charts shows mean±SEM; 1-way ANOVA; NS: non-significant, ****: p<0.0001). FIG. 9D depicts MTT cell proliferation assays in biological triplicates showing that scAAV SRSF1-shRNA10_GFP, H1-CPP1_GFP and H1-CPP2_GFP alleviates the cytotoxicity mediated by the expression of DPRs, but not when CPPs transcription is driven the RNAPII promoter. Bar charts shows mean±SEM; 1-way ANOVA; NS: non-significant, ****: p<0.0001.

### Example 4

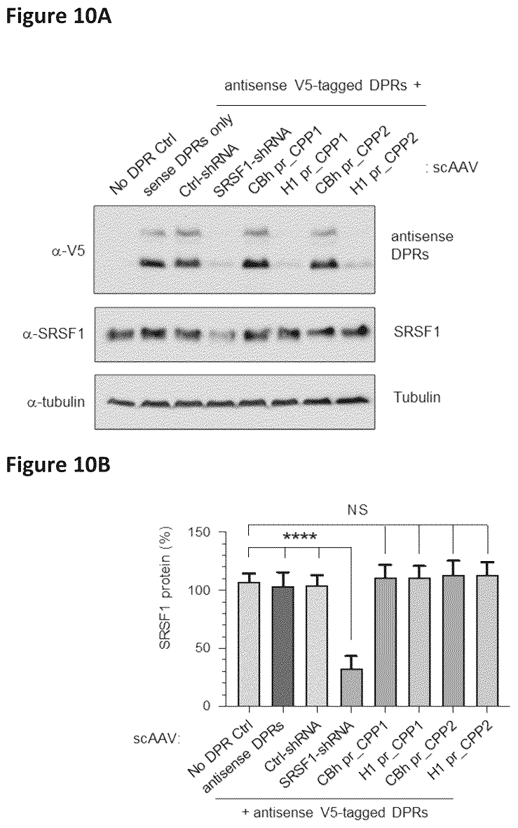

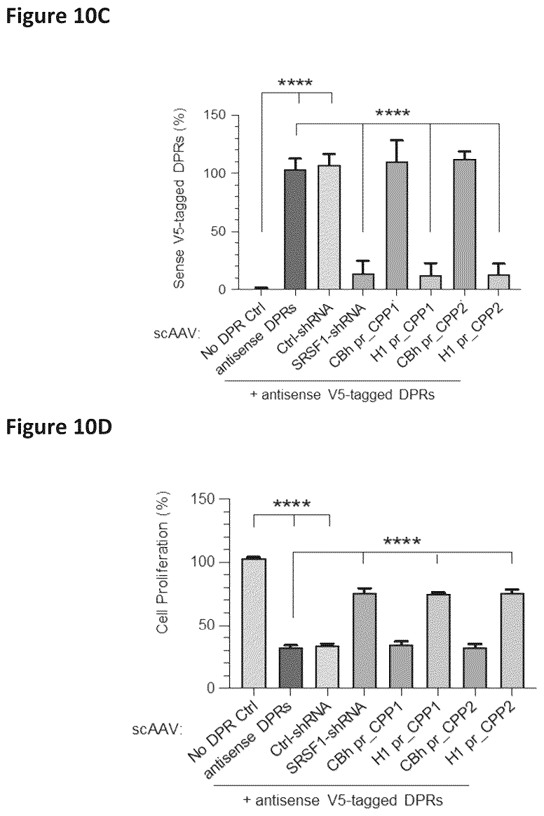

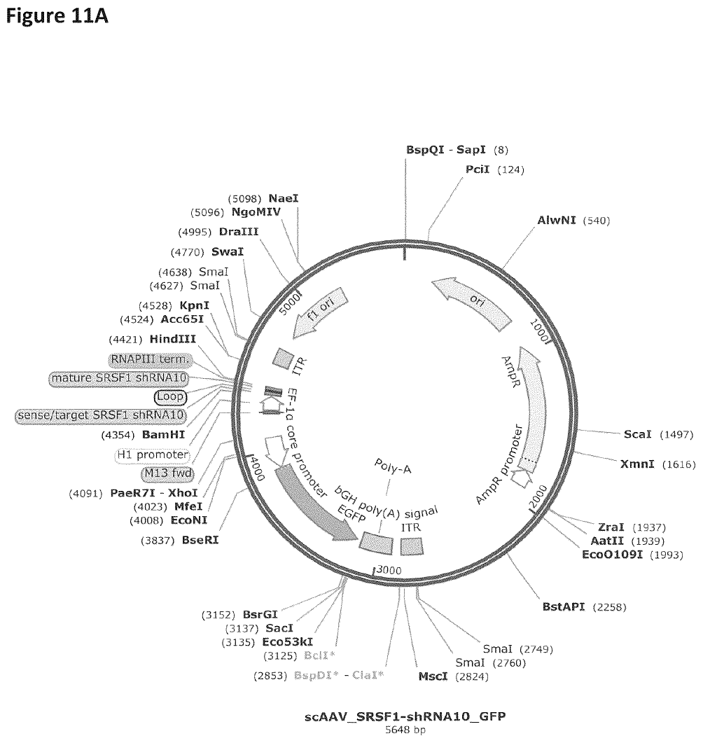

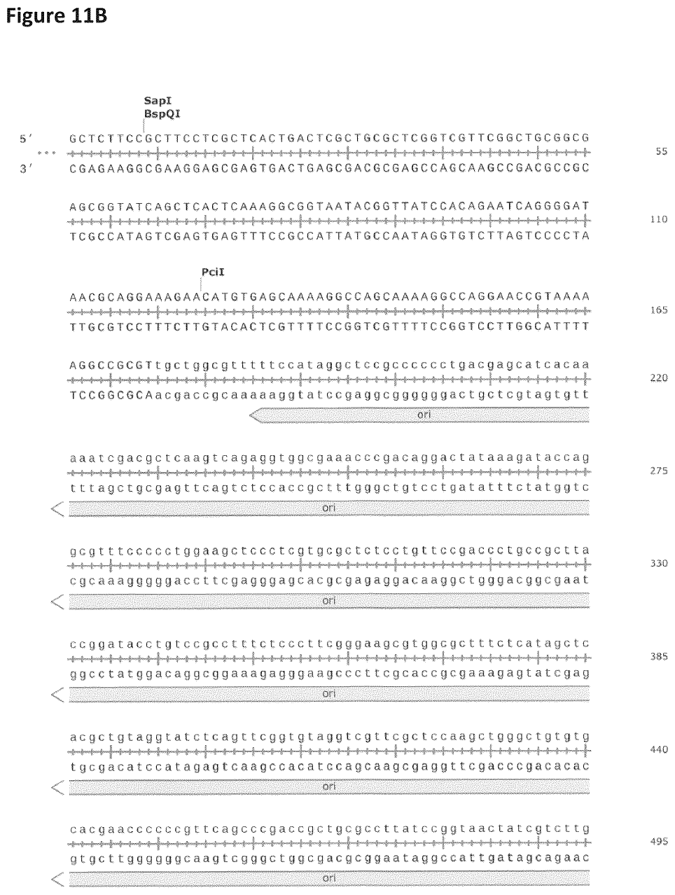

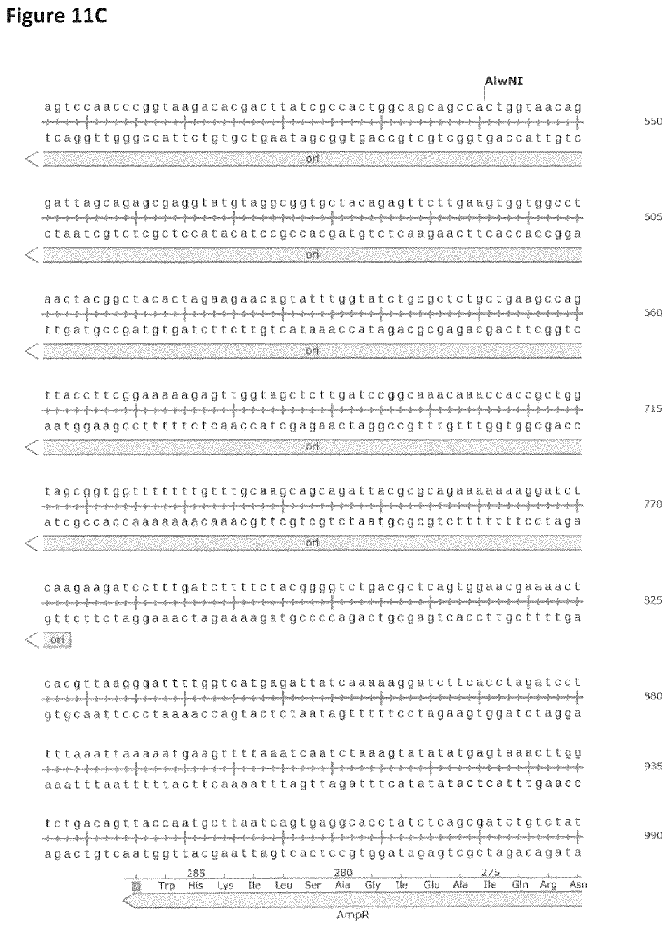

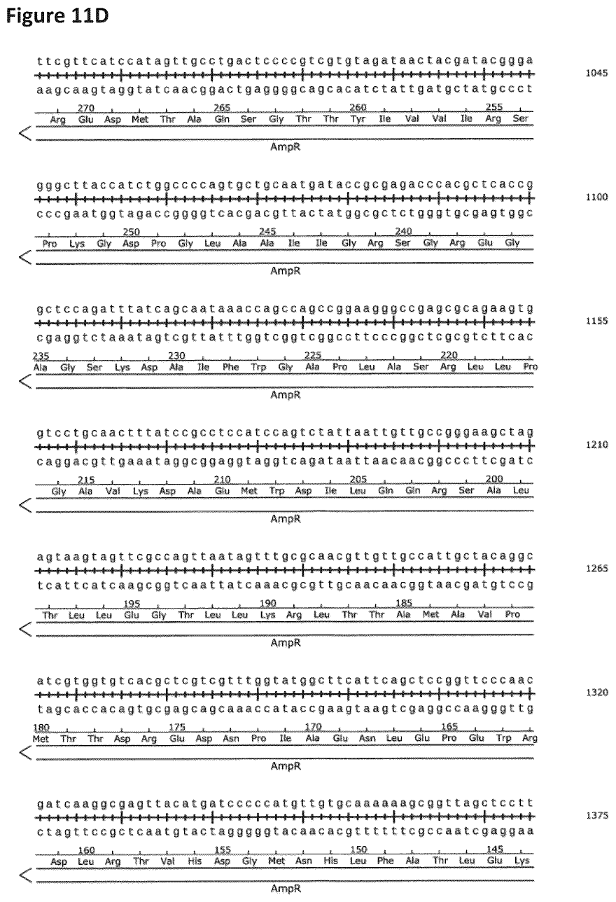

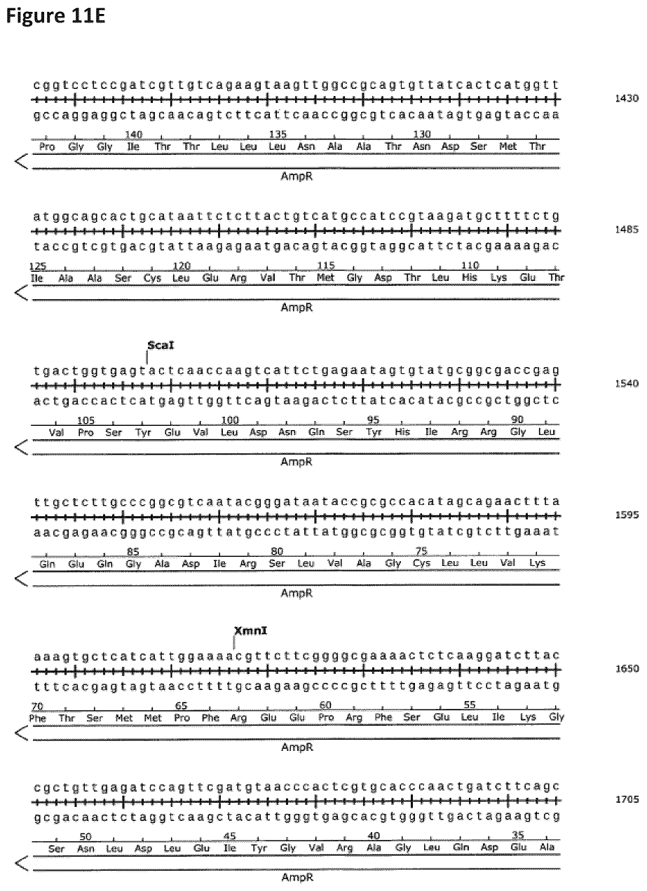

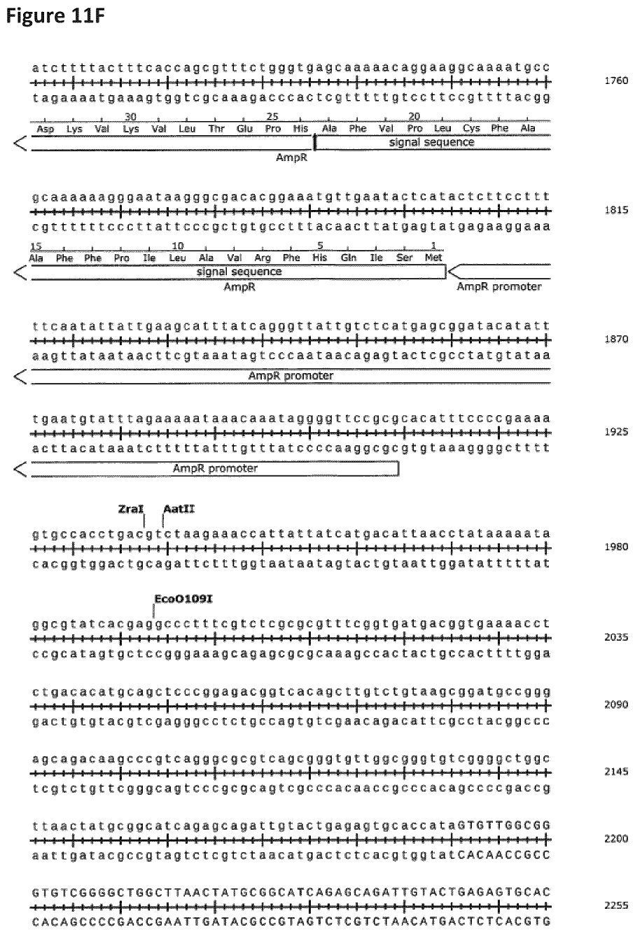

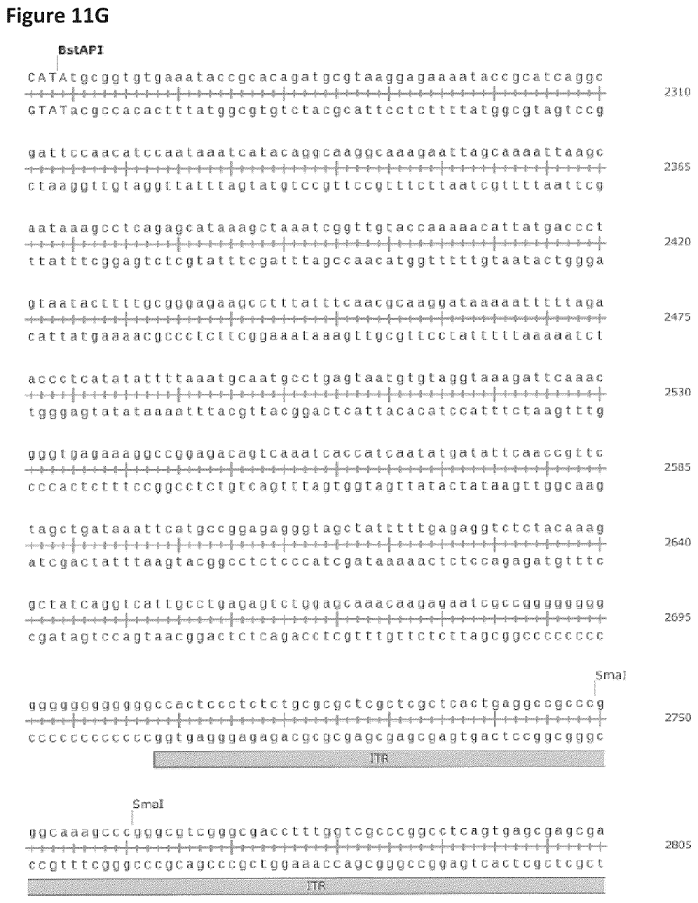

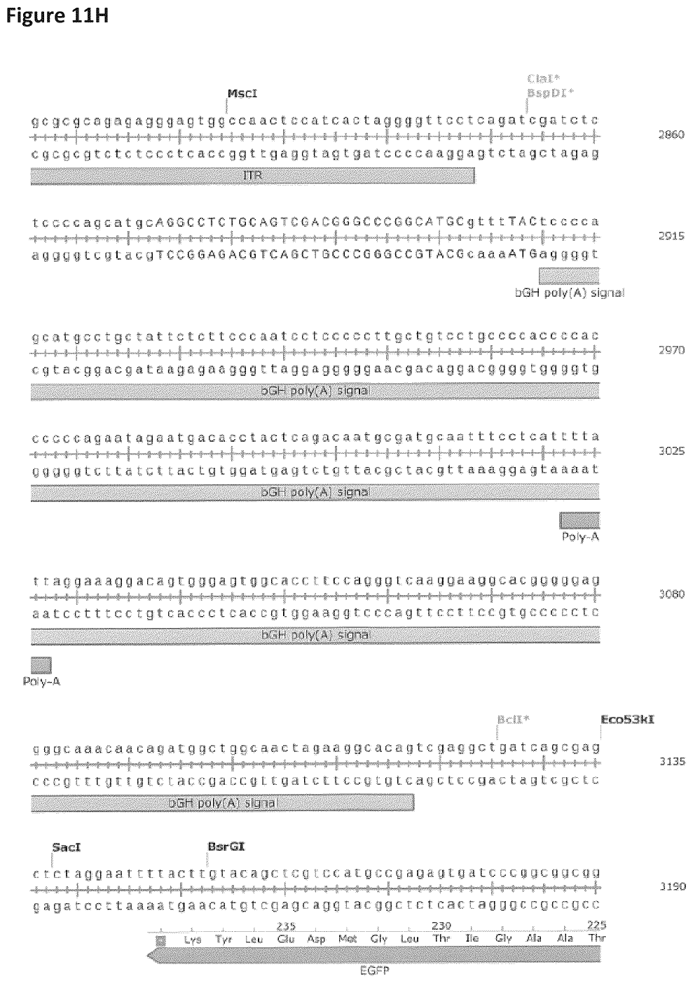

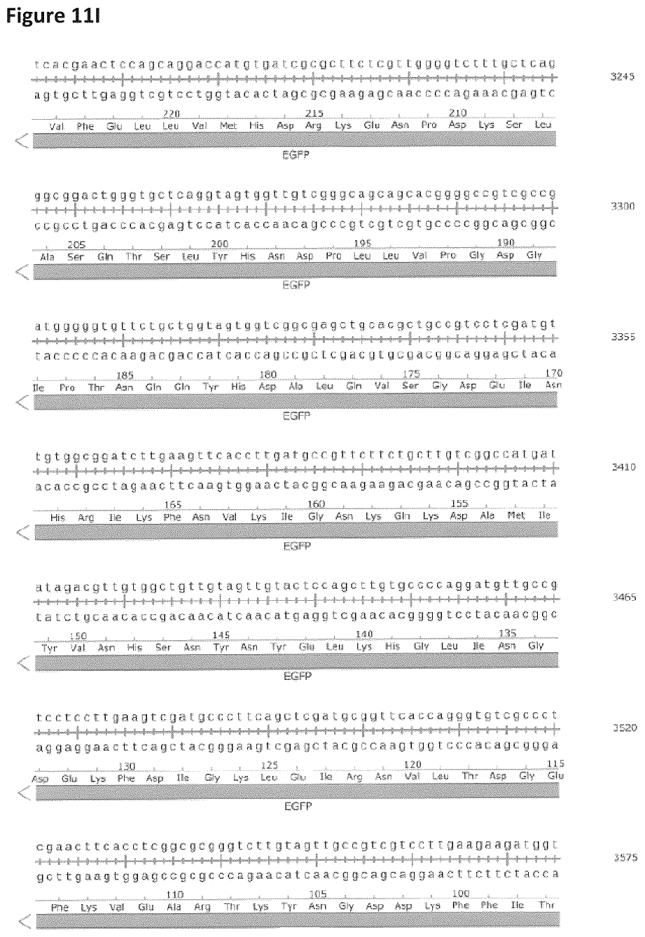

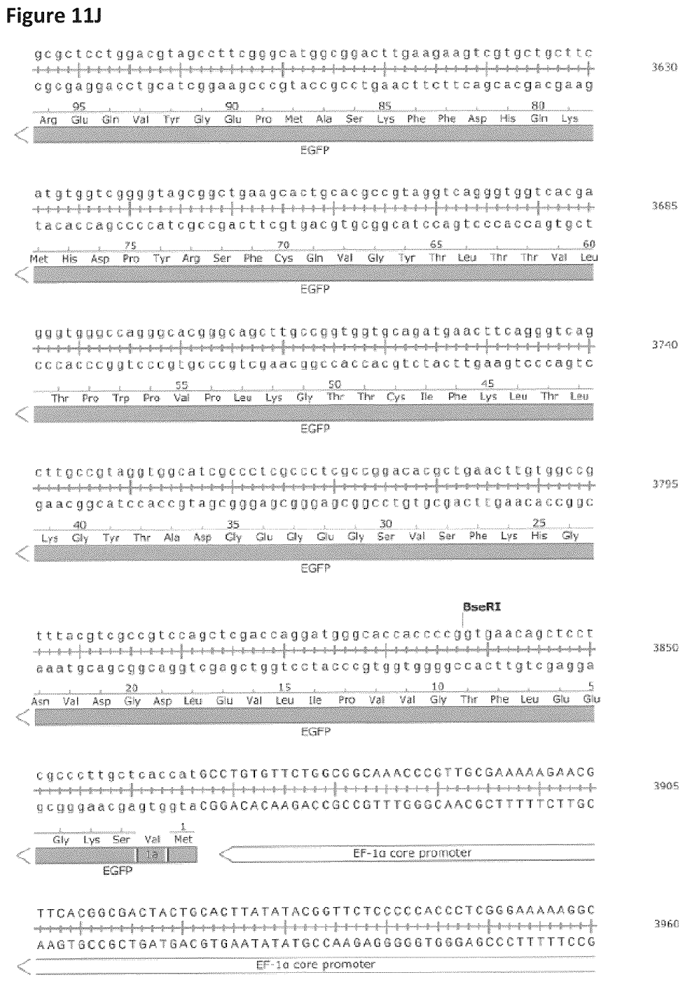

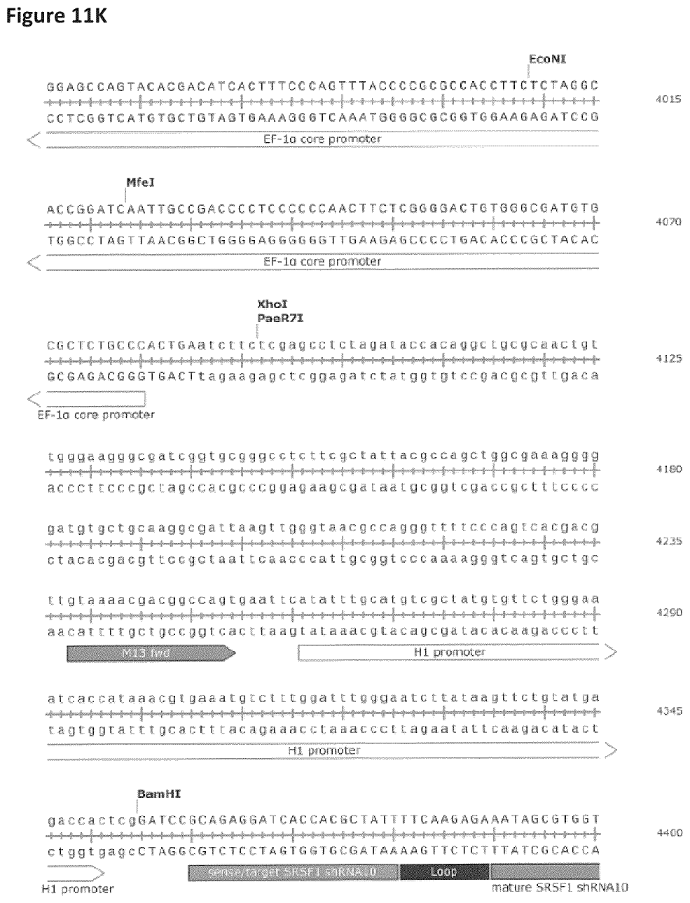

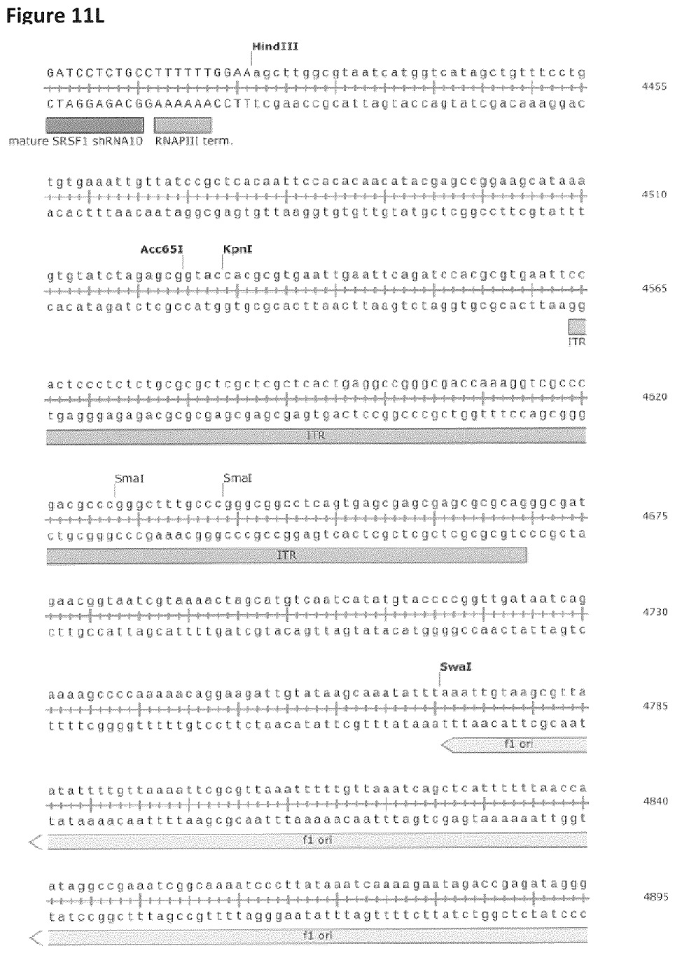

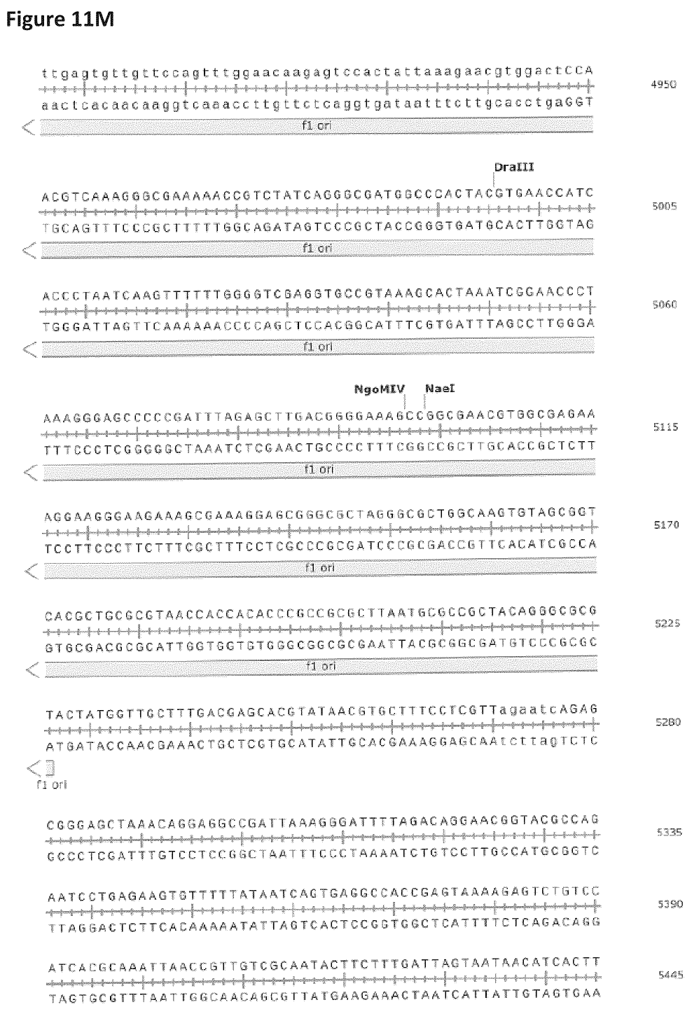

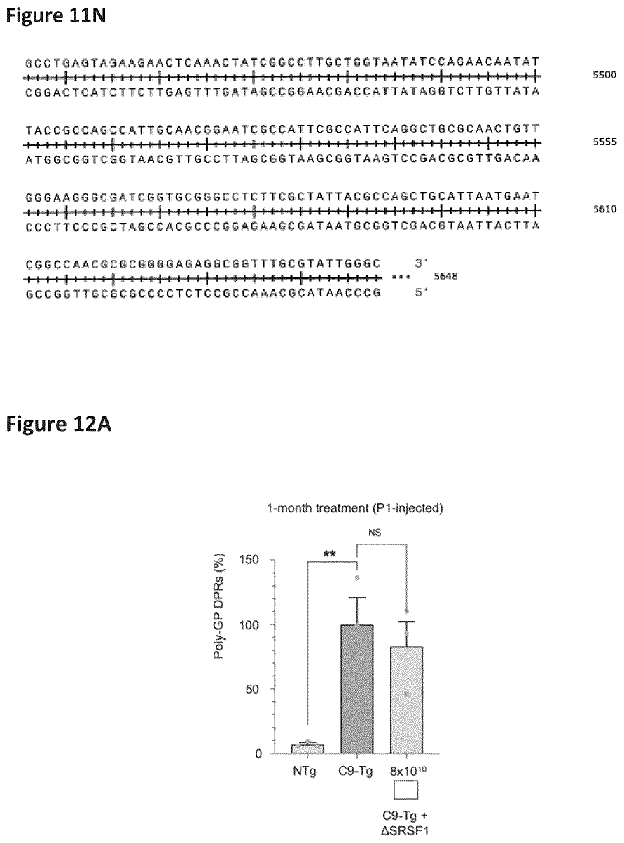

scAAV SRSF1-shRNA, CPP1 and CPP2 inhibits the production of antisense DPRs and rescue the DPR-associated cytotoxicity in a human cell model of C9ORF72-ALS/FTD. Human HEK293T cells were co-transfected with antisense G2C4×43 C9ORF72-repeat plasmid expressing antisense V5-tagged dipeptide-repeat protein (DPRs) in a RAN translation manner and scAAV plasmids expressing SRSF1-shRNA10 or 2 different cell permeable peptides (CPP1: SRSF1 aa89-120 CPP (SEQ ID NO 59) and CPP2: SRSF1 aa132-144 CPP (SEQ ID NO 75)). We tested potential expression under mammalian ubiquitous RNA polymerase II (CBh) or RNA polymerase Ill (H1) promoters. FIG. 10A Western blots show depletion of SRSF1 and inhibition of the RAN translation of antisense DPRs upon co-transfection with scAAV SRSF1-shRNA10_GFP, H1-CPP1_GFP and H1-CPP2_GFP, but not when CPPs transcription is driven the RNAPII promoter. SRSF1 and DPRs expression levels are quantified in triplicate biological experiments in FIGS. 10B and 10C respectively (Bar charts shows mean±SEM; 1-way ANOVA; NS: non-significant, ****: p<0.0001). Note that there is no expression of CPP1 or CPP2 from the protein-coding CBh promoter. FIG. 10D depicts MTT cell proliferation assays in biological triplicates showing that scAAV SRSF1-shRNA10_GFP, H1-CPP1_GFP and H1-CPP2_GFP alleviates the cytotoxicity mediated by the expression of DPRs, but not when CPPs transcription is driven the RNAPII promoter. Bar charts shows mean±SEM; 1-way ANOVA; NS: non-significant, ****: p<0.0001.

### Example 5

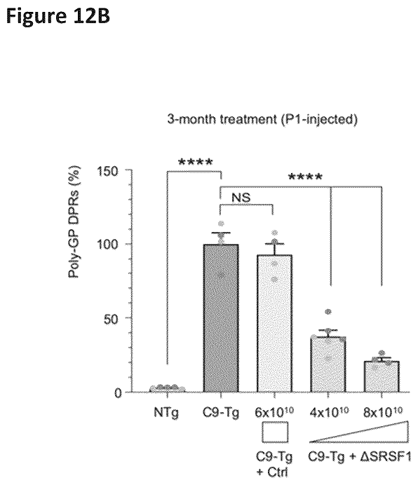

The data for SRSF1-shRNA (FIGS. 12A-B) shows that the svAAV9-SRSF1-shRNA10 virus leads to inhibition of the DPRs in mouse brains and complements the data showing that it leads to SRSF1 depletion in mouse brains.

### Example 6

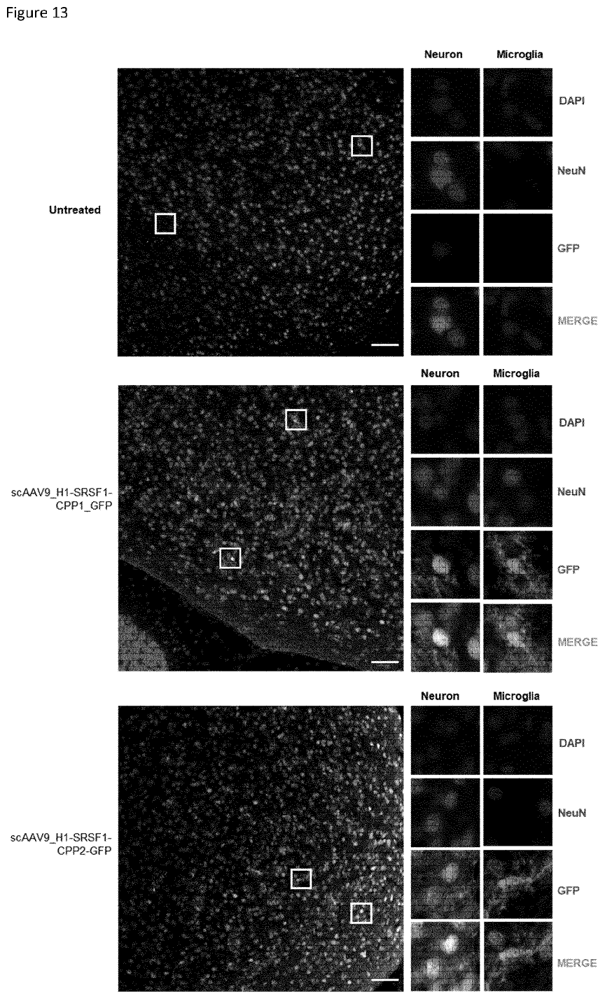

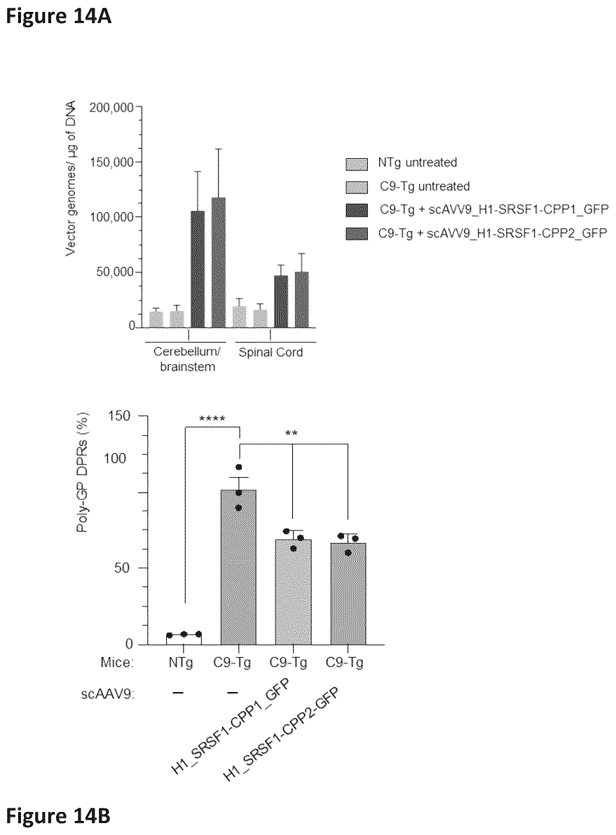

The data shows that the scAAV9 virus expressing CPP1 or CPP2 and co-expressing GFP efficiently transduced neuronal and glial cells in mouse brains (FIG. 13) and leads to DPR inhibition (FIGS. 14A-14B).

1. A viral vector comprising a transcription cassette for the expression of a nucleic acid molecule in a mammalian host cell wherein said nucleic acid molecule is operably linked to a promoter adapted to express said nucleic acid molecule in said mammalian host cell wherein said vector comprises a non-expressed nucleotide sequence and wherein said nucleic acid molecule encodes an antagonistic agent that targets Serine/Arginine Rich Splice Factor (SRSF1).

2. (canceled)

3. The viral vector according to claim 1 wherein said antagonistic agent:
is a nucleic acid molecule comprising a nucleic acid sequence encoding a polypeptide or peptide;
is a nucleic acid-based agent; or
is a nucleic acid-based agent comprising an antisense nucleic acid, an inhibitory RNA, a shRNA, or miRNA molecule that is complementary to and inhibits the expression of a nucleic acid encoding SRSF1.

4.-5. (canceled)

6. The viral vector according to claim 3, comprising the inhibitory RNA, wherein said inhibitory RNA comprises or consists of a nucleotide sequence as set forth in SEQ ID NO: 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58.

7. The viral vector according to claim 3, comprising the shRNA, wherein said shRNA comprises or consists of a nucleotide sequence selected from the group consisting of SEQ ID NOs: 2, 3, 4, 5, 6, 7, 8, 9, 10, and 11.

8.-10. (canceled)

11. The viral vector according to claim 3, comprising the nucleic acid molecule comprising the nucleic acid sequence encoding the peptide and wherein:
said peptide comprises an amino acid sequence that is at least 32 amino acids in length and comprises the amino acid sequence set forth in SEQ ID NO: 59;
said peptide is a dominant negative protein comprising a modification of the amino acid sequence set forth in SEQ ID NO: 60 or 61;
said modified protein comprises the amino acid sequence set forth in SEQ ID NO: 62 or 63;
said peptide comprises an amino acid sequence that is at least 10, 11, 12, 13, 14, 15, 20, 25, 30, 35, 40, or 42 amino acids in length and which is set forth in SEQ ID NO: 90; or
said peptide comprises the amino acid sequence set forth in SEQ ID NO: 75.

12.-14. (canceled)

15. The viral vector according to claim 3, comprising the nucleic acid sequence encoding the peptide, wherein said peptide comprises an amino acid sequence that is set forth in SEQ ID NO: 75.

16. The viral vector according to claim 1 wherein said viral vector comprises;
an RNA Pol III terminator, or
inverted terminal repeat nucleotide sequences; or
wherein said viral vector is an adeno-associated virus [AAV].

17. (canceled)

18. The viral vector according to claim 1 wherein said promoter is selected from the group consisting of H1 Polymerase III promoter, U6 promoter, U7 promoter and the mammalian 7SK promoter.

19.-20. (canceled)

21. The viral vector according to claim 16 wherein said viral vector is scAAV9 or scAAVrh10.

22. A pharmaceutical composition comprising a viral vector according to claim 1 and an excipient or carrier.

23. (canceled)

24. A method of treating a neurodegenerative disease, comprising administering the viral vector of claim 1 to a subject in need thereof, thereby treating the neurodegenerative disease.

25. The method according to claim 24 wherein said neurodegenerative disease is amyotrophic lateral sclerosis (ALS), sporadic or familial amyotrophic lateral sclerosis, ALS not caused by pathological C9ORF72-repeat expansions, sporadic frontotemporal dementia (FTD), or Fragile X-associated tremor/ataxia syndrome (FXTAS).

26.-29. (canceled)

30. A cell transfected with the viral vector of claim 1.

31. (canceled)

32. An isolated nucleic acid molecule encoding an shRNA molecule comprising or consisting of a nucleotide sequence selected from the group consisting of SEQ ID NOs: 2, 3, 4, 5, 6, 7, 8, 9, 10, and 11.

33.-35. (canceled)

36. A cell penetrating polypeptide comprising or consisting of an amino acid sequence set forth in SEQ ID NO: 90, wherein said polypeptide is between 13-42 amino acids in length.

37. (canceled)

38. The cell penetrating peptide according to claim 36 wherein said polypeptide comprises or consist of an amino acid sequence set forth in SEQ ID NO 75.

39. An antagonistic agent comprising a nucleic acid molecule wherein said nucleic acid molecule comprises a nucleic acid molecule which inhibits expression of SRSF1.

40. The agent according to claim 39 wherein:
said nucleic acid molecule is a double stranded nucleic acid molecule comprising a sense strand and an antisense strand comprising nucleotide sequences wherein said antisense nucleotide strand is adapted to anneal by complementary base pairing to a nucleic acid molecule encoding human SRSF1; or
said nucleic acid molecule is a single stranded nucleotide sequence comprising an antisense nucleotide sequence wherein said antisense nucleotide sequence is adapted to anneal by complementary base pairing to a nucleic acid molecule encoding SRSF1.

41.-46. (canceled)

47. The agent according to claim 39 wherein the nucleic acid molecule is at least 15 nucleotides in length.

48. The agent according to claim 47 wherein said antagonistic agent comprises a nucleic acid molecule comprising a nucleotide sequence set forth in SEQ ID NO: 67 wherein said nucleic acid molecule is a double stranded inhibitory RNA and is 19-23 nucleotides in length.

49. The agent according to claim 39 wherein said antagonistic agent comprises a nucleic acid molecule comprising modified nucleotides or modified sugars.

50.-53. (canceled)

54. The agent according to claim 49 wherein said antagonistic agent targets a nucleic acid sequence selected from the group consisting of SEQ ID NOs: 110, 111, 112, 113, 114, 115, 116, 117, 118, and 119.

55. A pharmaceutical composition comprising the agent according to claim 39 and an excipient or carrier.

56. (canceled)

57. A method of treating a neurodegenerative disease, comprising administering the agent according to claim 39 to a subject in need thereof, thereby treating the neurodegenerative disease.

58. The method according to claim 57 wherein said neurodegenerative disease is ALS, sporadic or familial amyotrophic lateral sclerosis, ALS not caused by pathological C9ORF72-repeat expansion, FTD, or FXTAS.

59.-62. (canceled)

63. A shRNA molecules comprising a nucleotide sequence, or variant thereof, selected from the group consisting of SEQ ID NOs: 91, 92, 93, 94, 95, 96, 97, 98, 99, and 100.

64.-66. (canceled)


SEQUENCE LISTING (119 sequences)

SEQ ID NO: 1 - DNA, 5651 bp, synthetic construct
  misc_feature 1..5651: note = artificial sequence
  source 1..5651: mol_type = other DNA
gctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgccgggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcgatctctccccagcatgcgttttacctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctaggaattttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgaatcttctcgagcctctagataccacaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcatatttgcatgtcgctatgtgttctgggaaatcaccataaacgtgaaatgtctttggatttgggaatcttataagttctgtatgagaccactcggatccgtttagtgaaccgtcagaagccaccatgggcagctggcaggatctgaaagatcatatgcgcgaagccggcggtgggaaaccgattcccaacccgctgctgggcctcgatagcaccggcggatatggtcgcaaaaagcgcagacagcgccggaggtaattttttaagcttggcgtaatcatggtcatagctgtttcctgtgtgaaattgttatccgctcacaattccacacaacatacgagccggaagcataaagtgtatctagagcggtaccacgcgtgaattgaattcagatccacgcgtgaattccactccctctctgcgcgctcgctcgctcactgaggccgggcgaccaaaggtcgcccgacgcccgggctttgcccgggcggcctcagtgagcgagcgagcgcgcagggcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcattaatgaatcggccaacgcgcggggagaggcggtttgcgtattgggc

SEQ ID NO: 2 - DNA, 21 bp, synthetic construct
  misc_feature 1..21: note = artificial sequence
  source 1..21: mol_type = other DNA
gctgatgtttaccgagatggc

SEQ ID NO: 3 - DNA, 21 bp, synthetic construct
  misc_feature 1..21: note = artificial sequence
  source 1..21: mol_type = other DNA
ggagtttgtacggaaagaaga

SEQ ID NO: 4 - DNA, 21 bp, synthetic construct
  misc_feature 1..21: note = artificial sequence
  source 1..21: mol_type = other DNA
ggaaagaagatatgacctatg

SEQ ID NO: 5 - DNA, 21 bp, synthetic construct
  misc_feature 1..21: note = artificial sequence
  source 1..21: mol_type = other DNA
gaaagaagatatgacctatgc

SEQ ID NO: 6 - DNA, 21 bp, synthetic construct
  misc_feature 1..21: note = artificial sequence
  source 1..21: mol_type = other DNA
gcctacatccgggttaaagtt

SEQ ID NO: 7 - DNA, 21 bp, synthetic construct
  misc_feature 1..21: note = artificial sequence
  source 1..21: mol_type = other DNA
gggcccagaagtccaagttat

SEQ ID NO: 8 - DNA, 21 bp, synthetic construct
  misc_feature 1..21: note = artificial sequence
  source 1..21: mol_type = other DNA
ggcccagaagtccaagttatg

SEQ ID NO: 9 - DNA, 21 bp, synthetic construct
  misc_feature 1..21: note = artificial sequence
  source 1..21: mol_type = other DNA
gcccagaagtccaagttatgg

SEQ ID NO: 10 - DNA, 21 bp, synthetic construct
  misc_feature 1..21: note = artificial sequence
  source 1..21: mol_type = other DNA
ggaagatctcgatctcgaagc

SEQ ID NO: 11 - DNA, 21 bp, synthetic construct
  misc_feature 1..21: note = artificial sequence
  source 1..21: mol_type = other DNA
gcagaggatcaccacgctatt

SEQ ID NO: 12 - DNA, 21 bp, synthetic construct
  misc_feature 1..21: note = artificial sequence
  source 1..21: mol_type = other DNA
aactgcctacatccgggttaa

SEQ ID NO: 13 - DNA, 21 bp, synthetic construct
  misc_feature 1..21: note = artificial sequence
  source 1..21: mol_type = other DNA
ttaaagttgatgggcccagaa

SEQ ID NO: 14 - DNA, 21 bp, synthetic construct
  misc_feature 1..21: note = artificial sequence
  source 1..21: mol_type = other DNA
gggcccagaagtccaagttat

SEQ ID NO: 15 - DNA, 21 bp, synthetic construct
  misc_feature 1..21: note = artificial sequence
  source 1..21: mol_type = other DNA
agcagaggatcaccacgctat

SEQ ID NO: 16 - DNA, 21 bp, synthetic construct
  misc_feature 1..21: note = artificial sequence
  source 1..21: mol_type = other DNA
tcaataatggaggcaatggta

SEQ ID NO: 17 - DNA, 21 bp, synthetic construct
  misc_feature 1..21: note = artificial sequence
  source 1..21: mol_type = other DNA
aatggtatgactccaagtgct

SEQ ID NO: 18 - DNA, 21 bp, synthetic construct
  misc_feature 1..21: note = artificial sequence
  source 1..21: mol_type = other DNA
gctaatttgtcacagtgctta

SEQ ID NO: 19 - DNA, 21 bp, synthetic construct
  misc_feature 1..21: note = artificial sequence
  source 1..21: mol_type = other DNA
gttaatgtgtgacctgctgtt

SEQ ID NO: 20 - DNA, 21 bp, synthetic construct
  misc_feature 1..21: note = artificial sequence
  source 1..21: mol_type = other DNA
actgctaaatctgcatgtcct

SEQ ID NO: 21 - DNA, 21 bp, synthetic construct
  misc_feature 1..21: note = artificial sequence
  source 1..21: mol_type = other DNA
tgatagagcgttgctatttca

SEQ ID NO: 22 - DNA, 21 bp, synthetic construct
  misc_feature 1..21: note = artificial sequence
  source 1..21: mol_type = other DNA
gggatcgaatcgctgtttcct

SEQ ID NO: 23 - DNA, 21 bp, synthetic construct
  misc_feature 1..21: note = artificial sequence
  source 1..21: mol_type = other DNA
gctgtttccttccgcttctct

SEQ ID NO: 24 - DNA, 21 bp, synthetic construct
  misc_feature 1..21: note = artificial sequence
  source 1..21: mol_type = other DNA
gctgatgtttaccgagatggc

SEQ ID NO: 25 - DNA, 21 bp, synthetic construct
  misc_feature 1..21: note = artificial sequence
  source 1..21: mol_type = other DNA
ggagtttgtacggaaagaaga

SEQ ID NO: 26 - DNA, 21 bp, synthetic construct
  misc_feature 1..21: note = artificial sequence
  source 1..21: mol_type = other DNA
ggaaagaagatatgacctatg

SEQ ID NO: 27 - DNA, 21 bp, synthetic construct
  misc_feature 1..21: note = artificial sequence
  source 1..21: mol_type = other DNA
agggagaaactgcctacatcc

SEQ ID NO: 28 - DNA, 21 bp, synthetic construct
  misc_feature 1..21: note = artificial sequence
  source 1..21: mol_type = other DNA
gggactaatgtgggaagaact

SEQ ID NO: 29 - DNA, 21 bp, synthetic construct
  misc_feature 1..21: note = artificial sequence
  source 1..21: mol_type = other DNA
gcaaccacgaaacctgtaata

SEQ ID NO: 30 - DNA, 21 bp, synthetic construct
  misc_feature 1..21: note = artificial sequence
  source 1..21: mol_type = other DNA
gggatcagattaccaggaaca

SEQ ID NO: 31 - DNA, 21 bp, synthetic construct
  misc_feature 1..21: note = artificial sequence
  source 1..21: mol_type = other DNA
gcatctgaaagataagcttct

SEQ ID NO: 32 - DNA, 21 bp, synthetic construct
  misc_feature 1..21: note = artificial sequence
  source 1..21: mol_type = other DNA
gcatctacgtgggtaacttac

SEQ ID NO: 33 - DNA, 21 bp, synthetic construct
  misc_feature 1..21: note = artificial sequence
  source 1..21: mol_type = other DNA
ggaacaacgattgccgcatct

SEQ ID NO: 34 - DNA, 21 bp, synthetic construct
  misc_feature 1..21: note = artificial sequence
  source 1..21: mol_type = other DNA
ggaagttggcaggatttaaag

SEQ ID NO: 35 - DNA, 21 bp, synthetic construct
  misc_feature 1..21: note = artificial sequence
  source 1..21: mol_type = other DNA
ggtaggttatacacgtattct

SEQ ID NO: 36 - DNA, 21 bp, synthetic construct
  misc_feature 1..21: note = artificial sequence
  source 1..21: mol_type = other DNA
gcaggtgatgtatgttatgct

SEQ ID NO: 37 - DNA, 21 bp, synthetic construct
  misc_feature 1..21: note = artificial sequence
  source 1..21: mol_type = other DNA
gctgatgtttaccgagatggc

SEQ ID NO: 38 - DNA, 21 bp, synthetic construct
  misc_feature 1..21: note = artificial sequence
  source 1..21: mol_type = other DNA
gaagttggcaggatttaaagg

SEQ ID NO: 39 - DNA, 21 bp, synthetic construct
  misc_feature 1..21: note = artificial sequence
  source 1..21: mol_type = other DNA
gaaagaagatatgacctatgc

SEQ ID NO: 40 - DNA, 21 bp, synthetic construct
  misc_feature 1..21: note = artificial sequence
  source 1..21: mol_type = other DNA
gcgtgaagcaggtgatgtatg

SEQ ID NO: 41 - DNA, 21 bp, synthetic construct
  misc_feature 1..21: note = artificial sequence
  source 1..21: mol_type = other DNA
gtaagttacccacgtagatgc

SEQ ID NO: 42 - DNA, 21 bp, synthetic construct
  misc_feature 1..21: note = artificial sequence
  source 1..21: mol_type = other DNA
agatgcggcaatcgttgttcc

SEQ ID NO: 43 - DNA, 21 bp, synthetic construct
  misc_feature 1..21: note = artificial sequence
  source 1..21: mol_type = other DNA
tcttctttccgtacaaactcc

SEQ ID NO: 44 - DNA, 21 bp, synthetic construct
  misc_feature 1..21: note = artificial sequence
  source 1..21: mol_type = other DNA
ctttaaatcctgccaacttcc

SEQ ID NO: 45 - DNA, 21 bp, synthetic construct
  misc_feature 1..21: note = artificial sequence
  source 1..21: mol_type = other DNA
agaatacgtgtataacctacc

SEQ ID NO: 46 - DNA, 21 bp, synthetic construct
  misc_feature 1..21: note = artificial sequence
  source 1..21: mol_type = other DNA
agcataacatacatcacctgc

SEQ ID NO: 47 - DNA, 21 bp, synthetic construct
  misc_feature 1..21: note = artificial sequence
  source 1..21: mol_type = other DNA
gccatctcggtaaacatcagc

SEQ ID NO: 48 - DNA, 21 bp, synthetic construct
  misc_feature 1..21: note = artificial sequence
  source 1..21: mol_type = other DNA
cctttaaatcctgccaacttc

SEQ ID NO: 49 - DNA, 21 bp, synthetic construct
  misc_feature 1..21: note = artificial sequence
  source 1..21: mol_type = other DNA
gcataggtcatatcttctttc

SEQ ID NO: 50 - DNA, 21 bp, synthetic construct
  misc_feature 1..21: note = artificial sequence
  source 1..21: mol_type = other DNA
catacatcacctgcttcacgc

SEQ ID NO: 51 - DNA, 22 bp, synthetic construct
  misc_feature 1..22: note = artificial sequence
  source 1..22: mol_type = other DNA
gcgctatccgcgacatcgacct

SEQ ID NO: 52 - DNA, 22 bp, synthetic construct
  misc_feature 1..22: note = artificial sequence
  source 1..22: mol_type = other DNA
tggccccgcagggaacaacgat

SEQ ID NO: 53 - DNA, 22 bp, synthetic construct
  misc_feature 1..22: note = artificial sequence
  source 1..22: mol_type = other DNA
tcgcgacggctatgattacgat

SEQ ID NO: 54 - DNA, 22 bp, synthetic construct
  misc_feature 1..22: note = artificial sequence
  source 1..22: mol_type = other DNA
cggcgctatccgcgacatcgac

SEQ ID NO: 55 - DNA, 22 bp, synthetic construct
  misc_feature 1..22: note = artificial sequence
  source 1..22: mol_type = other DNA
agggaacaacgattgccgcatc

SEQ ID NO: 56 - DNA, 22 bp, synthetic construct
  misc_feature 1..22: note = artificial sequence
  source 1..22: mol_type = other DNA
gcggtgtatggtcgcgacggct

SEQ ID NO: 57 - DNA, 22 bp, synthetic construct
  misc_feature 1..22: note = artificial sequence
  source 1..22: mol_type = other DNA
ctgcgggtggagtttcctcgaa

SEQ ID NO: 58 - DNA, 22 bp, synthetic construct
  misc_feature 1..22: note = artificial sequence
  source 1..22: mol_type = other DNA
gtggagtttcctcgaagcggcc

SEQ ID NO: 59 - protein, 32 aa, synthetic construct
  REGION 1..32: note = artificial sequence
PRSGRGTGRGGGGGGGGGAPRGRYGPPSRRSE

SEQ ID NO: 60 - protein, 248 aa, Homo sapiens
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

SEQ ID NO: 61 - protein, 201 aa, Homo sapiens
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEVGYTRILFFDQNWIQWS

SEQ ID NO: 62 - protein, 196 aa, synthetic construct
  REGION 1..196: note = artificial sequence
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPASGAGTGRGGGGGGGGGAPRGRYGPPSAASENRVVVSGLPPSGSWQDLKDHMREAGDVCYADVYRDGTGVVEFVRKEDMTYAVRKLDNTKFRSHEGETAYIRVKVDG

SEQ ID NO: 63 - protein, 187 aa, synthetic construct
  REGION 1..187: note = artificial sequence
MAGNNDCRIYVGNLPPDIRTKDIEDVFYKYGAIRDIDLKNRRGGPPFAFVEFEDPRDAEDAVYGRDGYDYDGYRLRVEFPASGAGTGRGGGGGGGGGAPRGRYGPPSAASENRVVVSGLPPSGSWQDLKDHMREAGDVCYADVYRDGTGVVEFVRKEDMTYAVRKLDNTKFRSHEGETAYIRVKVDG

SEQ ID NO: 64 - DNA, 141 bp, synthetic construct
  misc_feature 1..141: note = artificial sequence
  source 1..141: mol_type = other DNA
ccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 65 - DNA, 91 bp, synthetic construct
  misc_feature 1..91: note = artificial sequence
  source 1..91: mol_type = other DNA
atatttgcatgtcgctatgtgttctgggaaatcaccataaacgtgaaatgtctttggatttgggaatcttataagttctgtatgagaccac

SEQ ID NO: 66 - DNA, 5648 bp, synthetic construct
  misc_feature 1..5648: note = artificial sequence
  source 1..5648: mol_type = other DNA
gctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgccggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcgatctctccccagcatgcaggcctctgcagtcgacgggcccggcatgcgttttactccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctaggaattttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgaatcttctcgagcctctagataccacaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcatatttgcatgtcgctatgtgttctgggaaatcaccataaacgtgaaatgtctttggatttgggaatcttataagttctgtatgagaccactcggatccgcagaggatcaccacgctattttcaagagaaatagcgtggtgatcctctgccttttttggaaagcttggcgtaatcatggtcatagctgtttcctgtgtgaaattgttatccgctcacaattccacacaacatacgagccggaagcataaagtgtatctagagcggtaccacgcgtgaattgaattcagatccacgcgtgaattccactccctctctgcgcgctcgctcgctcactgaggccgggcgaccaaaggtcgcccgacgcccgggctttgcccgggcggcctcagtgagcgagcgagcgcgcagggcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcattaatgaatcggccaacgcgcggggagaggcggtttgcgtattgggc

SEQ ID NO: 67 - DNA, 300 bp, synthetic construct
  misc_feature 1..300: note = artificial sequence
  source 1..300: mol_type = other DNA
gctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataa

SEQ ID NO: 68 - DNA, 67 bp, synthetic construct
  misc_feature 1..67: note = artificial sequence
  source 1..67: mol_type = other DNA
gatccgggcccagaagtccaagttatttcaagagaataacttggacttctgggccccttttttggaa

SEQ ID NO: 69 - DNA, 67 bp, synthetic construct
  misc_feature 1..67: note = artificial sequence
  source 1..67: mol_type = other DNA
agctttccaaaaaaggggcccagaagtccaagttattctcttgaaataacttggacttctgggcccg

SEQ ID NO: 70 - DNA, 67 bp, synthetic construct
  misc_feature 1..67: note = artificial sequence
  source 1..67: mol_type = other DNA
gatccggaagatctcgatctcgaagcttcaagagagcttcgagatcgagatcttcccttttttggaa

SEQ ID NO: 71 - DNA, 67 bp, synthetic construct
  misc_feature 1..67: note = artificial sequence
  source 1..67: mol_type = other DNA
agctttccaaaaaagggaagatctcgatctcgaagctctcttgaagcttcgagatcgagatcttccg

SEQ ID NO: 72 - DNA, 67 bp, synthetic construct
  misc_feature 1..67: note = artificial sequence
  source 1..67: mol_type = other DNA
gatccgcagaggatcaccacgctattttcaagagaaatagcgtggtgatcctctgccttttttggaa

SEQ ID NO: 73 - DNA, 67 bp, synthetic construct
  misc_feature 1..67: note = artificial sequence
  source 1..67: mol_type = other DNA
agctttccaaaaaaggcagaggatcaccacgctatttctcttgaaaatagcgtggtgatcctctgcg

SEQ ID NO: 74 - DNA, 5692 bp, synthetic construct
  misc_feature 1..5692: note = artificial sequence
  source 1..5692: mol_type = other DNA
gctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgccgggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcgatctctccccagcatgcgttttacctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctaggaattttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgaatcttctcgagcctctagataccacaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcatatttgcatgtcgctatgtgttctgggaaatcaccataaacgtgaaatgtctttggatttgggaatcttataagttctgtatgagaccactcggatccgaagccaccatgccgcgcagcggccgcggcaccggccgcggtgggggcggcggtggaggtggcggagccccgagaggccgctatggaccgcccagccgccggagcgaaggcggtgggaaaccgattcccaacccgctgctgggcctcgatagcaccggcggatatggtcgcaaaaagcgcagacagcgccggaggtaattttttaagcttggcgtaatcatggtcatagctgtttcctgtgtgaaattgttatccgctcacaattccacacaacatacgagccggaagcataaagtgtatctagagcggtaccacgcgtgaattgaattcagatccacgcgtgaattccactccctctctgcgcgctcgctcgctcactgaggccgggcgaccaaaggtcgcccgacgcccgggctttgcccgggcggcctcagtgagcgagcgagcgcgcagggcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcattaatgaatcggccaacgcgcggggagaggcggtttgcgtattgggc

SEQ ID NO: 75 - protein, 13 aa, synthetic construct
  REGION 1..13: note = Cell Penetrating peptide
GSWQDLKDHMREA

SEQ ID NO: 76 - DNA, 5343 bp, Homo sapiens
  source 1..5343: mol_type = genomic DNA
gccattttggaggggccgcgggagacgtggtgccgctgcgggctcgctctgccgtgcgctaggcttggtgggaaggcctgttctcgagtccgcgcttttcgtcaccgccatgtcgggaggtggtgtgattcgtggccccgcagggaacaacgattgccgcatctacgtgggtaacttacctccagacatccgaaccaaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataagatgattggtgacactttttgtagaacccatgttgtatacagttttcctttattcagtacaatcttttcattttttaattcaaactgttttgttcagaatgggctaaagtgttgaattgcattcttgtaatatccccttgctcctaacatctacattcccttcgtgtctttgataaattgtattttaagtgatgtcatagacaggattgtttaaatttagttaactccatactcttcagactgtgatattgtgtaaatgtctatctgccctggtttgtgtgaactgggatgttgggggtgtttgtggttatcttacctggggaagttcttatgtttatcttgcttttcatgtgtctttctgtagacatatctgaagagatggattaagaatgctttggattaaggattgtggagcacatttcaatcattttaggattgtcaaaaggaggattgaggaggatcagatcaataatggaggcaatggtatgactccaagtgctattgtcacagatgaaattggcagtattgaccttatactaaaaggcaggggttaaaaatgattatatacattttccttaaaacacttgcaaacattttattcagttgtctttagctacaattgctttgctttttaaaccttggcaattgtggcaaaattatattgcccattttgtagcaacttattttgctcccttccccccatttttgttttaatagggactaatgtgggaagaactggctaatttgtcacagtgcttagttacaactgttaatgtgtgacctgctgttggtgtacatgtgggtacagggtgtttttaaatccaacaagatagagtataatatcaatactgctaaatctgcatgtcctctgtgtgactgatagagcgttgctatttcatttttttaagacaaaatgaaagcaaaatatagagttccaatgtattggtgtagataatctagttgggaatacttttaagtctcaccttcccctttaaactaatattcataattggttcatatgtttaaaagactttaatttacaaattaaattgcaaatgggagcattagatttagttttagacttaggtgggtagcaatgccagtaaacttaaattacgtaacttcttgcaaccacgaaacctgtaatacgctgtacagtaacaagtgttggcattatcagttgaactgtaaatacaaaatgcttcttccaattagtctctatgatgattaagtttctaaaatttatctgaacaccattcagaaacttgttttggggaatttgatagttattgatgtgcatctgttaaactgatgacagacataactcatcattccccagaaaccttttttgattacagtatctaacattttgcctcctcttttttggttttgctggttataaaggtttggattggagagggctcactggatcccaatccttggagctggatcattggattcaaatcataatgtggataggatagggaggatgaattaccaggattcatggagcgggatcagattaccaggaacataggagtggattcctgccccaaccaaaccgcattcgtgtggatttttttattcaacttaattggctattccaaagattttttttttcctatttttgacgattggagcccttaagatgcacgatggaattgtgttttgcgttttttggtaaaaggagcaaagcgaggacctggagataaacgctggagcaatctccttggaaggattcagcacgagtagatggtaaacatttaaaggggaaagggggggtttgtttaaaatagtaaatcagtaagtcacttctaaatttaaagaaaacaaaattggagttgaagaataagtaggtttccaattggctattgccgttttctttgaaaaaataaacattttttaaaaaactatgcatggttgtcctttttcctcttcatgtaagattctaactgggtctatcagttaatctttaaattgttaagtaagataagattttgactcttgtgttaatgtgttagcaaattaaaagttcttaaaaggcaatctaatggtattagccatcttttattgttaattgtaaaagtcttcaggggaaagcaaaaggggagaataaggcatttgtgtatgtaacttggtaaatgacggtgggggatggatctagcatctgaaagataagcttctctactttgttataaagtggttaaaaaactatagatgctgcttattttctggtggtcatagacaacataggcttttgtgcaaaattggttgatggctactaatgttcacttggagatagcttttgatattctcaatgaaactcatctcaaaaaaaggtaagtattaaatgttaacatcagcacagatgtattagaactgttttttgtttttgagacagagtctcgctctgttctccagactggagggcagtggtgtgatctaggctcactgcaacctccacccctggatttgagtgattctcgtgcctcagtctcccaagtagctgagactacaagtgtgtgccacccttgcccggctaattttgtatttttagtagagatgtggtttctctgtgttacccaggctggtcccaaaactcctggcctcaagtgatctgcctgccttggcctcccaaagtgttaggattacaggtgtgagccaccatgctcagcctgtagaacttttaacccaagtctcatttcttttttgaaagggaagagtgcacaagattaactgcttctttggatgaatcattgttaataaaaagctgggcatttagaattttgccttataagcccttctccaaccataagattattttgtaccaaaaactttggtgttctctaccaaagcagttaaaaacttttagcctgctacttcttgtatttgtctactgacagccccttggtactatttaggttgggggaggggacctaaaataaatagactttaacatttcccttgggtgctaatcatagttggaagttgaatttaaggtgattatttgggtgacaattaaaaacctaaggaaaaccagaaatcttggtagtggaagaaatgtgtaaggtcaccccaatcggtagattttaatgaacgttgtggaatgttgggaagaggggatgttaagttgaatgcagaatttcactaagtacttagtgtaagtttaaggatgtagctctttttatctaagaattcaatgtaatggccaaaaggcagatttactgtttaaaaatttgaataattttacatgacattcttgaaattctaagaagttttatgtgtagaacattttaaaaattcatcagattattaaagggaaaataaatgattaatgataattttggaaggttaatgtgagctagacttaagtaaactttggttcatttgtgttcattgaatgttttggaaatgaccaaaaaatgtaaatggccttcactcaagtttgagtgtttaaagttgaaagatgtgctctactaaaagttatagtaattctaacctcacattgaaatgagacagtattccttgttatacaggctgaatttgaagattagagaggatctaatgtttacttaggtaaaggggcataggttttgtagttaagatgaccagacagctaaaagctgtgatgggaagtatggactgctcctatttatagtctcagaaaatggacctttaggtctctatccgtattggcaattattagaagaaagttacacccttttgaaactacaaaagctgtcttggaatttccccctcttctccctatttatgtccccttagaatattttagggagcctataattattttctaaccaaggaaaaacttaagtctctttaagaagcaattacttttcataacatcagattgaataacccaccttgctgttcagcccacatcctactggaaacaaaaggtaagaaacccattttctggttcttgattgtttgggtctgaattttgtttttaaaactaagctaagtttaatgttttttaaaatgctgtttggaatatgaatagattccccgtaaaatgatttttcctaagttttatgctttagtaaatattcagtgctcacgtctgtgcatcatagtgcttgcgtttaatatgatttattgtagaatctcaacttttcttggtgtttgttgtctttgaaacattgtcttggtcattagggctggtgttttcacatttctgtggtcaaggtggatttcttatgtgtgcctttttgcttactttgtatatgaattttgtaatttaaattgcaagtaagttatatatatgtatttaccataaatagtattaaaagatgagaaactgttagactgaagttctgttgtaacataaccattatttccatcacagtatgaagactgcaaacgcagaaaacagattacagtctcttatccattttttgaaatccaaaaactacgaaaacaaaagattttctgttgttgagctaattaaatgtgaaccctgaccagaa

SEQ ID NO: 77 - RNA, 21 nt, synthetic construct
  misc_feature 1..21: note = artificial sequence
  source 1..21: mol_type = other RNA
gccatctcggtaaacatcagc

SEQ ID NO: 78 - RNA, 21 nt, synthetic construct
  misc_feature 1..21: note = artificial sequence
  source 1..21: mol_type = other RNA
tcttctttccgtacaaactcc

SEQ ID NO: 79 - RNA, 21 nt, synthetic construct
  misc_feature 1..21: note = artificial sequence
  source 1..21: mol_type = other RNA
cataggtcatatcttctttcc

SEQ ID NO: 80 - RNA, 21 nt, synthetic construct
  misc_feature 1..21: note = artificial sequence
  source 1..21: mol_type = other RNA
gcataggtcatatcttctttc

SEQ ID NO: 81 - RNA, 21 nt, synthetic construct
  misc_feature 1..21: note = artificial sequence
  source 1..21: mol_type = other RNA
aactttaacccggatgtaggc

SEQ ID NO: 82 - RNA, 21 nt, synthetic construct
  misc_feature 1..21: note = artificial sequence
  source 1..21: mol_type = other RNA
ataacttggacttctgggccc

SEQ ID NO: 83 - RNA, 21 nt, synthetic construct
  misc_feature 1..21: note = artificial sequence
  source 1..21: mol_type = other RNA
cataacttggacttctgggcc

SEQ ID NO: 84 - RNA, 21 nt, synthetic construct
  misc_feature 1..21: note = artificial sequence
  source 1..21: mol_type = other RNA
ccataacttggacttctgggc

SEQ ID NO: 85 - RNA, 21 nt, synthetic construct
  misc_feature 1..21: note = artificial sequence
  source 1..21: mol_type = other RNA
gcttcgagatcgagatcttcc

SEQ ID NO: 86 - RNA, 21 nt, synthetic construct
  misc_feature 1..21: note = artificial sequence
  source 1..21: mol_type = other RNA
aatagcgtggtgatcctctgc

SEQ ID NO: 87 - DNA, 21 bp, synthetic construct
  misc_feature 1..21: note = artificial sequence
  source 1..21: mol_type = other DNA
ttaaagttgatgggcccagaa

SEQ ID NO: 88 - DNA, 106 bp, synthetic construct
  misc_feature 1..106: note = artificial sequence
  source 1..106: mol_type = other DNA
ccactccctctctgcgcgctcgctcgctcactgaggccgggcgaccaaaggtcgcccgacgcccgggctttgcccgggcggcctcagtgagcgagcgagcgcgcag

SEQ ID NO: 89 - DNA, 126 bp, synthetic construct
  misc_feature 1..126: note = artificial sequence
  source 1..126: mol_type = other DNA
ggcagctggcaggatctgaaagatcatatgcgcgaagccggcggtgggaaaccgattcccaacccgctgctgggcctcgatagcaccggcggatatggtcgcaaaaagcgcagacagcgccggagg

SEQ ID NO: 90 - protein, 42 aa, synthetic construct
  REGION 1..42: note = artificial sequence
GSWQDLKDHMREAGGGKPIPNPLLGLDSTGGYGRKKRRQRRR

SEQ ID NO: 91 - RNA, 51 nt, synthetic construct
  misc_feature 1..51: note = shRNA
  source 1..51: mol_type = other RNA
gctgatgtttaccgagatggcttcaagagagccatctcggtaaacatcagc

SEQ ID NO: 92 - RNA, 51 nt, synthetic construct
  misc_feature 1..51: note = shRNA
  source 1..51: mol_type = other RNA
ggagtttgtacggaaagaagattcaagagatcttctttccgtacaaactcc

SEQ ID NO: 93 - RNA, 51 nt, synthetic construct
  misc_feature 1..51: note = shRNA
  source 1..51: mol_type = other RNA
ggaaagaagatatgacctatgttcaagagacataggtcatatcttctttcc

SEQ ID NO: 94 - RNA, 51 nt, synthetic construct
  misc_feature 1..51: note = shRNA
  source 1..51: mol_type = other RNA
gaaagaagatatgacctatgcttcaagagagcataggtcatatcttctttc

SEQ ID NO: 95 - RNA, 51 nt, synthetic construct
  misc_feature 1..51: note = shRNA
  source 1..51: mol_type = other RNA
gcctacatccgggttaaagttttcaagagaaactttaacccggatgtaggc

SEQ ID NO: 96 - RNA, 51 nt, synthetic construct
  misc_feature 1..51: note = shRNA
  source 1..51: mol_type = other RNA
gggcccagaagtccaagttatttcaagagaataacttggacttctgggccc

SEQ ID NO: 97 - RNA, 51 nt, synthetic construct
  misc_feature 1..51: note = shRNA
  source 1..51: mol_type = other RNA
ggcccagaagtccaagttatgttcaagagacataacttggacttctgggcc

SEQ ID NO: 98 - RNA, 51 nt, synthetic construct
  misc_feature 1..51: note = shRNA
  source 1..51: mol_type = other RNA
gcccagaagtccaagttatggttcaagagaccataacttggacttctgggc

SEQ ID NO: 99 - RNA, 51 nt, synthetic construct
  misc_feature 1..51: note = shRNA
  source 1..51: mol_type = other RNA
ggaagatctcgatctcgaagcttcaagagagcttcgagatcgagatcttcc

SEQ ID NO: 100 - RNA, 51 nt, synthetic construct
  misc_feature 1..51: note = shRNA
  source 1..51: mol_type = other RNA
gcagaggatcaccacgctattttcaagagaaatagcgtggtgatcctctgc

SEQ ID NO: 101 - DNA, 5651 bp, synthetic construct
  misc_feature 1..5651: note = scAAV-SRSF1 132-144 CPP_GFT 3'-5'
  source 1..5651: mol_type = other DNA
cgagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttatgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagagcgcgcaaagccactactgccacttttggagactgtgtacgtcgagggcctctgccagtgtcgaacagacattcgcctacggccctcgtctgttcgggcagtcccgcgcagtcgcccacaaccgcccacagccccgaccgaattgatacgccgtagtctcgtctaacatgactctcacgtggtatacgccacactttatggcgtgtctacgcattcctcttttatggcgtagtccgctaaggttgtaggttatttagtatgtccgttccgtttcttaatcgttttaattcgttatttcggagtctcgtatttcgatttagccaacatggtttttgtaatactgggacattatgaaaacgccctcttcggaaataaagttgcgttcctatttttaaaaatcttgggagtatataaaatttacgttacggactcattacacatccatttctaagtttgcccactctttccggcctctgtcagtttagtggtagttatactataagttggcaagatcgactatttaagtacggcctctcccatcgataaaaactctccagagatgtttccgatagtccagtaacggactctcagacctcgtttgttctcttagcggcccccccccccccccccccggtgagggagagacgcgcgagcgagcgagtgactccggcgggcccgtttcgggcccgcagcccgctggaaaccagcgggccggagtcactcgctcgctcgcgcgtctctccctcaccggttgaggtagtgatccccaaggagtctagctagagaggggtcgtacgcaaaatggaggggtcgtacggacgataagagaagggttaggagggggaacgacaggacggggtggggtggggggtcttatcttactgtggatgagtctgttacgctacgttaaaggagtaaaataatcctttcctgtcaccctcaccgtggaaggtcccagttccttccgtgccccctccccgtttgttgtctaccgaccgttgatcttccgtgtcagctccgactagtcgctcgagatccttaaaatgaacatgtcgagcaggtacggctctcactagggccgccgccagtgcttgaggtcgtcctggtacactagcgcgaagagcaaccccagaaacgagtcccgcctgacccacgagtccatcaccaacagcccgtcgtcgtgccccggcagcggctacccccacaagacgaccatcaccagccgctcgacgtgcgacggcaggagctacaacaccgcctagaacttcaagtggaactacggcaagaagacgaacagccggtactatatctgcaacaccgacaacatcaacatgaggtcgaacacggggtcctacaacggcaggaggaacttcagctacgggaagtcgagctacgccaagtggtcccacagcgggagcttgaagtggagccgcgcccagaacatcaacggcagcaggaacttcttctaccacgcgaggacctgcatcggaagcccgtaccgcctgaacttcttcagcacgacgaagtacaccagccccatcgccgacttcgtgacgtgcggcatccagtcccaccagtgctcccacccggtcccgtgcccgtcgaacggccaccacgtctacttgaagtcccagtcgaacggcatccaccgtagcgggagcgggagcggcctgtgcgacttgaacaccggcaaatgcagcggcaggtcgagctggtcctacccgtggtggggccacttgtcgaggagcgggaacgagtggtacggacacaagaccgccgtttgggcaacgctttttcttgcaagtgccgctgatgacgtgaatatatgccaagagggggtgggagccctttttccgcctcggtcatgtgctgtagtgaaagggtcaaatggggcgcggtggaagagatccgtggcctagttaacggctggggaggggggttgaagagcccctgacacccgctacacgcgagacgggtgacttagaagagctcggagatctatggtgtccgacgcgttgacaacccttcccgctagccacgcccggagaagcgataatgcggtcgaccgctttccccctacacgacgttccgctaattcaacccattgcggtcccaaaagggtcagtgctgcaacattttgctgccggtcacttaagtataaacgtacagcgatacacaagaccctttagtggtatttgcactttacagaaacctaaacccttagaatattcaagacatactctggtgagcctaggcaaatcacttggcagtcttcggtggtacccgtcgaccgtcctagactttctagtatacgcgcttcggccgccaccctttggctaagggttgggcgacgacccggagctatcgtggccgcctataccagcgtttttcgcgtctgtcgcggcctccattaaaaaattcgaaccgcattagtaccagtatcgacaaaggacacactttaacaataggcgagtgttaaggtgtgttgtatgctcggccttcgtatttcacatagatctcgccatggtgcgcacttaacttaagtctaggtgcgcacttaaggtgagggagagacgcgcgagcgagcgagtgactccggcccgctggtttccagcgggctgcgggcccgaaacgggcccgccggagtcactcgctcgctcgcgcgtcccgctacttgccattagcattttgatcgtacagttagtatacatggggccaactattagtcttttcggggtttttgtccttctaacatattcgtttataaatttaacattcgcaattataaaacaattttaagcgcaatttaaaaacaatttagtcgagtaaaaaattggttatccggctttagccgttttagggaatatttagttttcttatctggctctatcccaactcacaacaaggtcaaaccttgttctcaggtgataatttcttgcacctgaggttgcagtttcccgctttttggcagatagtcccgctaccgggtgatgcacttggtagtgggattagttcaaaaaaccccagctccacggcatttcgtgatttagccttgggatttccctcgggggctaaatctcgaactgcccctttcggccgcttgcaccgctctttccttcccttctttcgctttcctcgcccgcgatcccgcgaccgttcacatcgccagtgcgacgcgcattggtggtgtgggcggcgcgaattacgcggcgatgtcccgcgcatgataccaacgaaactgctcgtgcatattgcacgaaaggagcaatcttagtctcgccctcgatttgtcctccggctaatttccctaaaatctgtccttgccatgcggtcttaggactcttcacaaaaatattagtcactccggtggctcattttctcagacaggtagtgcgtttaattggcaacagcgttatgaagaaactaatcattattgtagtgaacggactcatcttcttgagtttgatagccggaacgaccattataggtcttgttataatggcggtcggtaacgttgccttagcggtaagcggtaagtccgacgcgttgacaacccttcccgctagccacgcccggagaagcgataatgcggtcgacgtaattacttagccggttgcgcgcccctctccgccaaacgcataacccg

SEQ ID NO: 102 - DNA, 5692 bp, synthetic construct
  misc_feature 1..5692: note = scAAV SRSF1 89-120 CPP_GFP 3'-5'
  source 1..5692: mol_type = other DNA
cgagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttatgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagagcgcgcaaagccactactgccacttttggagactgtgtacgtcgagggcctctgccagtgtcgaacagacattcgcctacggccctcgtctgttcgggcagtcccgcgcagtcgcccacaaccgcccacagccccgaccgaattgatacgccgtagtctcgtctaacatgactctcacgtggtatacgccacactttatggcgtgtctacgcattcctcttttatggcgtagtccgctaaggttgtaggttatttagtatgtccgttccgtttcttaatcgttttaattcgttatttcggagtctcgtatttcgatttagccaacatggtttttgtaatactgggacattatgaaaacgccctcttcggaaataaagttgcgttcctatttttaaaaatcttgggagtatataaaatttacgttacggactcattacacatccatttctaagtttgcccactctttccggcctctgtcagtttagtggtagttatactataagttggcaagatcgactatttaagtacggcctctcccatcgataaaaactctccagagatgtttccgatagtccagtaacggactctcagacctcgtttgttctcttagcggcccccccccccccccccccggtgagggagagacgcgcgagcgagcgagtgactccggcgggcccgtttcgggcccgcagcccgctggaaaccagcgggccggagtcactcgctcgctcgcgcgtctctccctcaccggttgaggtagtgatccccaaggagtctagctagagaggggtcgtacgcaaaatggaggggtcgtacggacgataagagaagggttaggagggggaacgacaggacggggtggggtggggggtcttatcttactgtggatgagtctgttacgctacgttaaaggagtaaaataatcctttcctgtcaccctcaccgtggaaggtcccagttccttccgtgccccctccccgtttgttgtctaccgaccgttgatcttccgtgtcagctccgactagtcgctcgagatccttaaaatgaacatgtcgagcaggtacggctctcactagggccgccgccagtgcttgaggtcgtcctggtacactagcgcgaagagcaaccccagaaacgagtcccgcctgacccacgagtccatcaccaacagcccgtcgtcgtgccccggcagcggctacccccacaagacgaccatcaccagccgctcgacgtgcgacggcaggagctacaacaccgcctagaacttcaagtggaactacggcaagaagacgaacagccggtactatatctgcaacaccgacaacatcaacatgaggtcgaacacggggtcctacaacggcaggaggaacttcagctacgggaagtcgagctacgccaagtggtcccacagcgggagcttgaagtggagccgcgcccagaacatcaacggcagcaggaacttcttctaccacgcgaggacctgcatcggaagcccgtaccgcctgaacttcttcagcacgacgaagtacaccagccccatcgccgacttcgtgacgtgcggcatccagtcccaccagtgctcccacccggtcccgtgcccgtcgaacggccaccacgtctacttgaagtcccagtcgaacggcatccaccgtagcgggagcgggagcggcctgtgcgacttgaacaccggcaaatgcagcggcaggtcgagctggtcctacccgtggtggggccacttgtcgaggagcgggaacgagtggtacggacacaagaccgccgtttgggcaacgctttttcttgcaagtgccgctgatgacgtgaatatatgccaagagggggtgggagccctttttccgcctcggtcatgtgctgtagtgaaagggtcaaatggggcgcggtggaagagatccgtggcctagttaacggctggggaggggggttgaagagcccctgacacccgctacacgcgagacgggtgacttagaagagctcggagatctatggtgtccgacgcgttgacaacccttcccgctagccacgcccggagaagcgataatgcggtcgaccgctttccccctacacgacgttccgctaattcaacccattgcggtcccaaaagggtcagtgctgcaacattttgctgccggtcacttaagtataaacgtacagcgatacacaagaccctttagtggtatttgcactttacagaaacctaaacccttagaatattcaagacatactctggtgagcctaggcttcggtggtacggcgcgtcgccggcgccgtggccggcgccacccccgccgccacctccaccgcctcggggctctccggcgatacctggcgggtcggcggcctcgcttccgccaccctttggctaagggttgggcgacgacccggagctatcgtggccgcctataccagcgtttttcgcgtctgtcgcggcctccattaaaaaattcgaaccgcattagtaccagtatcgacaaaggacacactttaacaataggcgagtgttaaggtgtgttgtatgctcggccttcgtatttcacatagatctcgccatggtgcgcacttaacttaagtctaggtgcgcacttaaggtgagggagagacgcgcgagcgagcgagtgactccggcccgctggtttccagcgggctgcgggcccgaaacgggcccgccggagtcactcgctcgctcgcgcgtcccgctacttgccattagcattttgatcgtacagttagtatacatggggccaactattagtcttttcggggtttttgtccttctaacatattcgtttataaatttaacattcgcaattataaaacaattttaagcgcaatttaaaaacaatttagtcgagtaaaaaattggttatccggctttagccgttttagggaatatttagttttcttatctggctctatcccaactcacaacaaggtcaaaccttgttctcaggtgataatttcttgcacctgaggttgcagtttcccgctttttggcagatagtcccgctaccgggtgatgcacttggtagtgggattagttcaaaaaaccccagctccacggcatttcgtgatttagccttgggatttccctcgggggctaaatctcgaactgcccctttcggccgcttgcaccgctctttccttcccttctttcgctttcctcgcccgcgatcccgcgaccgttcacatcgccagtgcgacgcgcattggtggtgtgggcggcgcgaattacgcggcgatgtcccgcgcatgataccaacgaaactgctcgtgcatattgcacgaaaggagcaatcttagtctcgccctcgatttgtcctccggctaatttccctaaaatctgtccttgccatgcggtcttaggactcttcacaaaaatattagtcactccggtggctcattttctcagacaggtagtgcgtttaattggcaacagcgttatgaagaaactaatcattattgtagtgaacggactcatcttcttgagtttgatagccggaacgaccattataggtcttgttataatggcggtcggtaacgttgccttagcggtaagcggtaagtccgacgcgttgacaacccttcccgctagccacgcccggagaagcgataatgcggtcgacgtaattacttagccggttgcgcgcccctctccgccaaacgcataacccg

SEQ ID NO: 103 - DNA, 5648 bp, synthetic construct
  misc_feature 1..5648: note = scAAV SRSF1-shRNA10-GFP 3'-5'
  source 1..5648: mol_type = other DNA
cgagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcttgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttatgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagagcgcgcaaagccactactgccacttttggagactgtgtacgtcgagggcctctgccagtgtcgaacagacattcgcctacggccctcgtctgttcgggcagtcccgcgcagtcgcccacaaccgcccacagccccgaccgaattgatacgccgtagtctcgtctaacatgactctcacgtggtatcacaaccgcccacagccccgaccgaattgatacgccgtagtctcgtctaacatgactctcacgtggtatacgccacactttatggcgtgtctacgcattcctcttttatggcgtagtccgctaaggttgtaggttatttagtatgtccgttccgtttcttaatcgttttaattcgttatttcggagtctcgtatttcgatttagccaacatggtttttgtaatactgggacattatgaaaacgccctcttcggaaataaagttgcgttcctatttttaaaaatcttgggagtatataaaatttacgttacggactcattacacatccatttctaagtttgcccactctttccggcctctgtcagtttagtggtagttatactataagttggcaagatcgactatttaagtacggcctctcccatcgataaaaactctccagagatgtttccgatagtccagtaacggactctcagacctcgtttgttctcttagcggccccccccccccccccccccggtgagggagagacgcgcgagcgagcgagtgactccggcgggcccgtttcgggcccgcagcccgctggaaaccagcgggccggagtcactcgctcgctcgcgcgtctctccctcaccggttgaggtagtgatccccaaggagtctagctagagaggggtcgtacgtccggagacgtcagctgcccgggccgtacgcaaaatgaggggtcgtacggacgataagagaagggttaggagggggaacgacaggacggggtggggtggggggtcttatcttactgtggatgagtctgttacgctacgttaaaggagtaaaataatcctttcctgtcaccctcaccgtggaaggtcccagttccttccgtgccccctccccgtttgttgtctaccgaccgttgatcttccgtgtcagctccgactagtcgctcgagatccttaaaatgaacatgtcgagcaggtacggctctcactagggccgccgccagtgcttgaggtcgtcctggtacactagcgcgaagagcaaccccagaaacgagtcccgcctgacccacgagtccatcaccaacagcccgtcgtcgtgccccggcagcggctacccccacaagacgaccatcaccagccgctcgacgtgcgacggcaggagctacaacaccgcctagaacttcaagtggaactacggcaagaagacgaacagccggtactatatctgcaacaccgacaacatcaacatgaggtcgaacacggggtcctacaacggcaggaggaacttcagctacgggaagtcgagctacgccaagtggtcccacagcgggagcttgaagtggagccgcgcccagaacatcaacggcagcaggaacttcttctaccacgcgaggacctgcatcggaagcccgtaccgcctgaacttcttcagcacgacgaagtacaccagccccatcgccgacttcgtgacgtgcggcatccagtcccaccagtgctcccacccggtcccgtgcccgtcgaacggccaccacgtctacttgaagtcccagtcgaacggcatccaccgtagcgggagcgggagcggcctgtgcgacttgaacaccggcaaatgcagcggcaggtcgagctggtcctacccgtggtggggccacttgtcgaggagcgggaacgagtggtacggacacaagaccgccgtttgggcaacgctttttcttgcaagtgccgctgatgacgtgaatatatgccaagagggggtgggagccctttttccgcctcggtcatgtgctgtagtgaaagggtcaaatggggcgcggtggaagagatccgtggcctagttaacggctggggaggggggttgaagagcccctgacacccgctacacgcgagacgggtgacttagaagagctcggagatctatggtgtccgacgcgttgacaacccttcccgctagccacgcccggagaagcgataatgcggtcgaccgctttccccctacacgacgttccgctaattcaacccattgcggtcccaaaagggtcagtgctgcaacattttgctgccggtcacttaagtataaacgtacagcgatacacaagaccctttagtggtatttgcactttacagaaacctaaacccttagaatattcaagacatactctggtgagcctaggcgtctcctagtggtgcgataaaagttctctttatcgcaccactaggagacggaaaaaacctttcgaaccgcattagtaccagtatcgacaaaggacacactttaacaataggcgagtgttaaggtgtgttgtatgctcggccttcgtatttcacatagatctcgccatggtgcgcacttaacttaagtctaggtgcgcacttaaggtgagggagagacgcgcgagcgagcgagtgactccggcccgctggtttccagcgggctgcgggcccgaaacgggcccgccggagtcactcgctcgctcgcgcgtcccgctacttgccattagcattttgatcgtacagttagtatacatggggccaactattagtcttttcggggtttttgtccttctaacatattcgtttataaatttaacattcgcaattataaaacaattttaagcgcaatttaaaaacaatttagtcgagtaaaaaattggttatccggctttagccgttttagggaatatttagttttcttatctggctctatcccaactcacaacaaggtcaaaccttgttctcaggtgataatttcttgcacctgaggttgcagtttcccgctttttggcagatagtcccgctaccgggtgatgcacttggtagtgggattagttcaaaaaaccccagctccacggcatttcgtgatttagccttgggatttccctcgggggctaaatctcgaactgcccctttcggccgcttgcaccgctctttccttcccttctttcgctttcctcgcccgcgatcccgcgaccgttcacatcgccagtgcgacgcgcattggtggtgtgggcggcgcgaattacgcggcgatgtcccgcgcatgataccaacgaaactgctcgtgcatattgcacgaaaggagcaatcttagtctcgccctcgatttgtcctccggctaatttccctaaaatctgtccttgccatgcggtcttaggactcttcacaaaaatattagtcactccggtggctcattttctcagacaggtagtgcgtttaattggcaacagcgttatgaagaaactaatcattattgtagtgaacggactcatcttcttgagtttgatagccggaacgaccattataggtcttgttataatggcggtcggtaacgttgccttagcggtaagcggtaagtccgacgcgttgacaacccttcccgctagccacgcccggagaagcgataatgcggtcgacgtaattacttagccggttgcgcgcccctctccgccaaacgcataacccg

SEQ ID NO: 104 - DNA, 747 bp, Homo sapiens
  source 1..747: mol_type = other DNA
atgtcgggaggtggtgtgattcgtggccccgcagggaacaacgattgccgcatctacgtgggtaacttacctccagacatccgaaccaaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataa

SEQ ID NO: 105 - DNA, 747 bp, Mus musculus
  source 1..747: mol_type = other DNA
atgtcgggaggtggtgtgatccgtggcccggcggggaacaacgactgccgcatctacgtgggtaacctacctccggatatccgaaccaaggacatcgaggacgtgttttacaaatacggcgccatccgcgacatcgacctgaagaaccgccgcgggggaccgcccttcgccttcgttgagttcgaggacccgcgagacgcggaagatgcggtgtacggtcgcgacggctacgactacgacggctaccggctgcgggtagagtttccccgaagcggccgcgggaccggccgaggcggcggcgggggtggaggcggcggcgccccgagaggccgctatggcccgccgtccaggcggtccgagaacagagtggttgtctctggactgcctccgagtggaagctggcaggacttaaaggatcacatgcgtgaggcaggtgatgtatgttacgctgatgtttaccgagatggcactggtgtcgtggagtttgtacggaaagaagatatgacgtatgcagttcgaaaactggataacactaagtttagatctcacgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataa

SEQ ID NO: 106 - protein, 286 aa, synthetic construct
  REGION 1..286: note = Amino acid sequence for ampicillin resistance
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 107 - protein, 239 aa, synthetic construct
  REGION 1..239: note = Amino acid sequence for GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 108 - protein, 62 aa, synthetic construct
  REGION 1..62: note = Amino acid sequence for SRSF1 89-120 cell permeable peptide
MPRSGRGTGRGGGGGGGGGAPRGRYGPPSRRSEGGGKPIPNPLLGLDSTGGYGRKKRRQRRR

SEQ ID NO: 109 - protein, 43 aa, synthetic construct
  REGION 1..43: note = Amino acid sequence for SRSF1 132-144 cell permeable peptide
MGSWQDLKDHMREAGGGKPIPNPLLGLDSTGGYGRKKRRQRRR

SEQ ID NO: 110 - DNA, 25 bp, synthetic construct
  misc_feature 1..25: note = nucleic acid target sequence
  source 1..25: mol_type = other DNA
tggcactggtgtcgtggagtttgta

SEQ ID NO: 111 - DNA, 25 bp, synthetic construct
  misc_feature 1..25: note = nucleic acid target sequence
  source 1..25: mol_type = other DNA
tggtgtcgtggagtttgtacggaaa

SEQ ID NO: 112 - DNA, 25 bp, synthetic construct
  misc_feature 1..25: note = nucleic acid target sequence
  source 1..25: mol_type = other DNA
tcgtggagtttgtacggaaagaaga

SEQ ID NO: 113 - DNA, 25 bp, synthetic construct
  misc_feature 1..25: note = nucleic acid target sequence
  source 1..25: mol_type = other DNA
aagatatgacctatgcagttcgaaa

SEQ ID NO: 114 - DNA, 25 bp, synthetic construct
  misc_feature 1..25: note = nucleic acid target sequence
  source 1..25: mol_type = other DNA
gagaaactgcctacatccgggttaa

SEQ ID NO: 115 - DNA, 25 bp, synthetic construct
  misc_feature 1..25: note = nucleic acid target sequence
  source 1..25: mol_type = other DNA
cgggttaaagttgatgggcccagaa

SEQ ID NO: 116 - DNA, 25 bp, synthetic construct
  source 1..25: mol_type = other DNA
tgatgggcccagaagtccaagttat

SEQ ID NO: 117 - DNA, 25 bp, synthetic construct
  misc_feature 1..25: note = nucleic acid target sequence
  source 1..25: mol_type = other DNA
cagaagtccaagttatggaagatct

SEQ ID NO: 118 - DNA, 25 bp, synthetic construct
  misc_feature 1..25: note = nucleic acid target sequence
  source 1..25: mol_type = other DNA
gagaagcagaggatcaccacgctat

SEQ ID NO: 119 - DNA, 25 bp, synthetic construct
  misc_feature 1..25: note = nucleic acid target sequence
  source 1..25: mol_type = other DNA
cgtcatagcagatctcgctctcgta